SEQUENCE LISTING (3 sequences)

SEQ ID NO: 1 - DNA, 7264 bp, Bos taurus
atgagtgaggaatgggattcaaactctacagaaaactggcattacatttggaataatgccacaacacatgatctgtactcagatatcaatattacctatgtgaactactatcttcaccagcctcaagtggcagcgattttcattatttcctactttttgatcttcttcctatgcatggtgggaaacacagtggtttgcttcattgtaatgaggaacaaacatatgcacacagtcactaatctcttcatcttgaacctggccataagtgatctactagttggtatattctgtatgcctatcacactgctggacaatattatagcaggtatgttgatccactccagtattcttgcctggaaaatcccatggatggaggagcctggtgggctacagtctatggggtcacaaacagctggaaatgactgagtgacttcacttatgttgatttgtgtacagctcaaagataatataaaaaaatatttgtcccatatccctgcagctatggtacagtcatccattcatttcaaatatttacggagttccaagaacttctccaagtagctgtcctcatgaggcctacattataaaggaggataaaaaaacaacaaacaaaaaactatataaacagagaataaaaagaattatggggaaaagtaaagcaagtgacagagatgagatgtggaggctgatttttatagagttcactgacggtcatccatgaatgatgacacttcttactgaagactatgaatttccttggcagttctgagcacatatagtatggtaggaatgttattgagactatatgcatcataaagctctaagaactgctaagtgtggtttccattaatatgatgtcttcaatataacgtaaatagatatttagaccctcttgtggttagctgggcttctctggtggttgggagatttccaacagtttttgatggaaggcaagcagcaggaccaatgatatgtcacaaagtggtagtttcattcatggagtagtaatttacatgtgcaacataaacaatggttcgggtgctaccctagaggacttccaggtccgtattaccacttcctaacacaactttatgtccctctcttgtggctcagctggtaaagaatccacctgtaatcaatccctgggttgggaagattcccctggaggatgacatggcaacccactctagttttcttgtctggaaaatccccatggacagaagagcctggcaggctgcagtccatggggtcacaaacagttggacacaactgagcgaccaagcacaaaacatcacattatataccccagaagtataggaatggtgtatctatggctcctggtagagttttggtacatagtacctgattaataaatatttgttgtacaaactaatgaatagcactcaagatactcatattccaaatctgtataagaaaatataaaaagtatttagatcaaacaagccatatcatggggctactgtggtggctcaggagtaaagaatttgcctgcaatgcaggagatgcagagatgtgggttcaatccctgggtcggaaagattccctgaaggagaaaatggcaacccactccagtaatcttgcccagaaagtaaactgatgttgaatgccacaaaagggaaagaactgtggtgtggtttgttgttactgctgtgtagtcagacacgactgagtgactaaacaataataacacaagtcatatcacagttcttttccattatggcattcaacataggtttactgaaaaatggagatttaagaatttatttctgtttctttcctttctctgaagtgggagtcagggaatgtttgagtggctattctatcataatatactacataaattctgtgtttccatgatgcttgtcatttaaaagcaatatttattaatgatgtacatttaaaaaaaatgatgtacatttttaagatgtgctagacaaaaagagttgataaaaattgttgtctcaataaacttaagaaatgatctcaatatgtctcccataaatatctataattaaattactagttaagttttttcatatacagtatccttccctacccctgattcctattcccaggaggcagccacattcagcatttttgcatttatttttggtaattactataatatttctgaataacatgtttttattctagtatattatccaactgcagaagatgcaatttagttctcattatcctcttctaccccaagagatagtttccctcaccaaactccactgaactgacagcactagggcaaaagaatgtaaatccatagaaactgtctgaatgtgaaattggaaaaacaacatgactggttgaaatttggtataaataccaacagacacatttatacagagccacaaatatacattcatttttcacctccctcattctctcaatatgagcacgtcattgttttttgttaaatcaatatttagggtatgcattactattattatatgtaccttacccctgctgaaccatgtagagtactatgatagcaaccttttctcttataaaatgtttttgttttcctggatttaataagggcataatcttttgatttgtttaatgttttgagtatagctatcaataatgttttctcagattttcttccaggagagttaagtttcttgccaataccttcaaacatataaagtacatatatgtgtttttaacacatcaaaaagatgtaaatgaggtgaataataaagcttccaagcttgttgtggggattagatatgttaatagatgcaaaatatttattagagcatatagaatgttgaaactactgtataagctttgacattattaatatactgaaaaacaaagctctaaatatattaatgaaaataatgggaaatgttgattgttccctggatcttttaggaaacagttacatgcatctaatttcatgtctttctcttcaaaatttcagtgaaattaaaatatacatgtatgatctctctgaagactaactgttccatttccctttcaggatggccttttggaagtacaatgtgcaagatcagtggcttggttcagggaatatctgttgcggcttctgtctttactttagttgcaatagcagtggataggtaggtcaaccccaaactctgaatccagaaaattgagcatgtctgcaactattctacctaaccagtgaaaaatgtgtcatctactacatttgggcatatctgtttaaaattgtattcataatatatccttttatatatatatatatatgtagtatataatatatatacatatgcatagtatatatgtgtgtgtatatatatttgtgcattatatatacataaattgtatccacagtatgtatccctttatatatatatatatatagagagagagagagagggagatagggtgtatgcatgtgcatgctcagttactcagtcatttctgattctttgtgaccacatggactgtggcctgccaagttcctctgtccatggaattttctaggcaagaatactggagtgggttgccatttcctactccaggggatcttcctgagccagggatcaaacccatgcctcctgcatttgcaggagcattctttaccactgcaccacctgagaaaccacacacacacacacacacactaagagttcagtaataaaataaaactagtaaagttttcatattttaaaattaaataattagagatgattcatgtcctagtttggcctgctataacaaggtataatacgttatttgatgaatgaataaatgaaaaaataatacttgaagtttccataattgttttacaaaaggagcaaaaatacctagaacagcacctatgaagttatccgtaatttaagggtgagtaaatgggagaattcactgattagaagactagatgaacacttggaggttaagacagaagacctatcacttcatggaaaatagatgggaacaaagtagaaacagtggcagattttattttcttggattccaaaatcactgtggatggtgaccacagccacgaaatgaaatgatgcttgcttcttggaagttacaagggaagcctggtgacaaacctatacagtgtattacaaagcagagacatcactttgtggacaaaactcacatagtccaacctatggtttttccagtagtcctctagggatgtgagagttggaccatgaagaaggctgagagccaaagaattgatgctttagaactgtgctgctggagaagactcttgagagttccttggactgcaaagagatcaaaccagtcaatcctaaaggaaatcaaccgtgaatattcattggaaggactgatgctgaagctgaaactacaattgatgtgaagaaccaactcattggaaaacactctgatgctgggaaagattgagggcaggaggagaagtgggtgacagagaattagatggttggagagcttcaccgactcaatggagatgaaattgaacaaactctgggagatagtggaggacagagaagcctagcgtggtgcagtccatggggttgcaaagagctgaacacaacttagcaactgagcaacaacaaaaacaagactttacatatgctttgaaggagttgtaaagaaagacaacagagtagtaaaagctcaagctaactagtcgttatataaagatattagataaattagtttgggttgcttctaagccatttaaaaactctgttttcttacctgcagatctggaaaacagtaagtttcataacatttcagttttatagagtcatcaaaaaaatcctagaaaattcaatagatgataatactttgaaaaatgtgttatgcagttgcatagttgtatggttatcttatactgcagaaggaaatggcaacccagtccagtattcttgcctggaaaattccatggaaagaggagcctggcaggctacagttcatgaggtcacaaagagtcagacatgactgaatgactgagcacatggttatcttataatgaacataatgaacatcaataataacattaagaatcacaatgacaaaaattaacagcagtaaaatgaaccagtgttactcttcatattgatgttgaattttcatgctccttagaagatatggaacaccaggaaggtgtataaacagaactcataattggcaactctcagagtcttacagctctgaaaaaaaccaccaagacacttggtggctcaaaacagcagtgttcagtacttcccacaactctgtagattggctgggtgtggttctcctactctatgtcttatagctgaaattgctcatgcttccacttataccatgcttgctaatgttcaactaactggccaaagcaaattgcatgtccaagcacacagatcatatgtgaggggaccacagaagggcatgaaggaaagtataagaatatgggctctggagccaaaccacatgtgcaacaatcatgtgtgattatgggcaagaatttttaccctttctaagacttttccccataaaaggcttaaagatacaatccatgcaaaccaatgaaaaggaccttagaacagaatattaaatgttcaatatgggctgcttaacactaacatttttattataactttaaaatttttattggagtagagttgatttacaatgttgtgttaatttctgctatacagaaaagtgaatcagttgtaaatacatatgcatacatccgtgctttttttctaaaggtttattgtatttatttatttaatttactttttggctgtgctgggtcttcgttgctgtgcataggcttttctctaactgcagcgagtggggctactctccgttgtgatgcacaggcttcttgttgcagcagcttctcttgttacggagcacaggatctaggtgcgcaggtttcagtagctgcagcacatgggctctgtaattgtggttcacaggctctagacgctggctcagtagttgtgatgcatcaacttagccactctgcggcatgtgagatcctcccagaccagggatcaaaccagcatcccttgcactgcaagacggattcctaatcgctggaccaccagggaagcctgagtacttttactattaatagtgtctgatatactccacttattcgtattttgagttgaaattaatctcatataataattacagaaaatgcgtctctcctaattctaactttctacattttagggagaacgtggatgaagactgcagttactgaaatttaattaatgactcagccagaagttatgagcagtccttcactgatatttgcctttcgttacaggttccggtgtgtcatctacccttttaaaccaaagctcactatcaagacggcgtttgtcatcattatgattatctgggtcctggccattgccatcatgtccccatctgcagtaatgttacatgtacaggaagaaaaaaattaccgagtgagattcaactcccaggataaaaccagcccagtctactggtgccgggaagactggccaagtcaggaaatgaggaggatttataccacagtgctgtttgccaatatctacctggctcccctgtccctcattgtcatcatgtatggaaggattggaatttcactgttcaagaggaaagtgccccacacaggcaaacagaaccgggagcagtggcatgtggtatccaagaagaagcagaagatcattaagatgctcctgaccgtggctctgcttttcattctctcctggttgcccctgtggaccctgatgatgctctcagattatgttgacctgtctgcaaatgaactgcaggtcatcaatatctacatctacccttttgcacactggctggccttctgcaacagcagcgtcaaccccatcatttatggtttcttcaatgaaaattttcgtcgtggtttccaagatgcttttcacctccagctctgccaaaaaagagcaaagtccaaggaagtctacactctgagagctaaaaacactgtggtcatcaacacatctcatctgtcagcacaggaatcaacagttaaaaacccacacgaggaaactgtgctttgtaggataagtgctgaaaagcccttacaggaattaatgatggaagaattaggagaaattaccagtagcaatgagatgtaaaaagagctggtgtgatgattttaactctgctgtgtgatatatattgaaatattgttgatgtctatggcttcgttctttagttctttctatgaatgttagaaaccctctctgaaaaaaagtcaacaaaatgaacc

SEQ ID NO: 2 - DNA, 801 bp, Bos taurus
tagattgggaggactggggctggcatggcttgggctgagtggatttggatggcgaactttcagctcgggggrcctggtgctgagtgggcttggtttgagtgggtttgggttgaaagggcacaggttgggagggtttgtgctgggactgtttgacctggggtgacttgtgctgattaggtctgaattgagagctgggcaaatacatgctgtaggacataggatggaatcccatttggaaagatggatttgaaggcccaccttgtgtcttcagcttagctccttgctgaggggcagtccttcttggtttttgtgtggctcctgctgcttgaaaggcttgatgatgaggattttcaatatggggttttgttctcatggatgttctcactgtctctgttggcttygttcccctggcactgacttgcccaggcttcccaactgctcctcctggccatgaacccagggaatggaggtgacctacctgggagtcctctttcccagacctttcaagggttcctattgtctgtggtctctgaggccaggccggtatatgctgagacatgggtcttggtggtctcccaaaagttctacccatgtgatgtctccaaggagttcctgaaaatctcataaatgtttcacctgaataaaatctctggggctggaaatattgaattccaaaacgtttgcctggactatgtgcccttgtattctgaaagggcaaaggatggaacctcttaggcctctgctgtaaccagaagckggagcccatagcccaaggggctttcaaagaaacatggttaaagt

SEQ ID NO: 3 - DNA, 801 bp, Bos taurus
ccttattaactgcgtattgcatggactagcatcygtatacaattgaagtcttcagtgtgctaaacctgtaggagcctgggtttgacattgtggcccaaatatctgaatagttgggtgtttatgtgcttcagtgatagaggtgctccatccctgcagtttacacagagtggcarcgattcccagaaaaatttacaggcaggagyttcagcctcattttccataccagcattgctttcacggctcatggatctgaaggattgcattgagaacatctagtcctattgcactctcagaaactgtgggaaaagtcatattcttaaaccttcatgcaacttgtattcttgttggaaattagtcctgtgatttcttagttgtcttcatactggccatatttaaagaayatcacagtccttttttgtacttgaataattagatgtagtttagtgaaggagacatgtgaatgttttcttccaaaaggaatttggaatcagttttaacgagtttgaaataaaagtgctccctaacctgttaatatgcagaaaatattatctcaaatttttctactgctgaggcacataatctgataaaactttttttttttttcttctgtttaaggtagtttttactgttttctgttctgaaccatgttaaaatttgtatatcttttataacatasatttccccccttattttgaaagtataaaattgggcatctcaaaagtcaaatgtgggatcattagttaatcactaagactaggcacataatggaaattcagtcaggttttttattgactgagtccc

(19) United States
(12) Patent Application Publication
(10) Pub. No.: US 2015/0240308 A1
(43) Pub. Date: Aug. 27, 2015

(54) Genetic Markers for Mastitis Resistance

(71) Applicant: Aarhus Universitet, Århus C (DK)

(72) Inventors: Bernt Guldbrandtsen, Tjele (DK); Goutam Sahana, Tjele (DK); Mogens Sandø Lund, Tjele (DK); Bo Thomsen, Arhus V (DK); Christian Bendixen, Ulstrup (DK); Frank Bernd Panitz, Silkeborg (DK)

(21) Appl. No.: 14/424,466

(22) PCT Filed: Aug. 28, 2013
(86) PCT No.: PCT/EP2013/067838

§ 371 Date: Feb. 27, 2015

(30) Foreign Application Priority Data
Aug. 28, 2012 (DK) PA 2012 70508

(51) Int. Cl. C12Q 1/68

(52) U.S. Cl. CPC C12Q 1/6883; C12Q 2600/16; C12Q 2600/156; C12Q 2600/112

(57) ABSTRACT

A method is provided for determining resistance to mastitis in a bovine subject, comprising detecting in a sample from said bovine subject the presence or absence of at least one genetic marker that is associated with at least one trait indicative of mastitis resistance of said bovine subject and/or off-spring therefrom. Furthermore, methods are provided for determining a breeding value in respect of susceptibility to mastitis in a bovine subject, comprising detecting in a sample from said bovine subject the presence or absence of at least one genetic marker that is associated with at least one trait indicative of mastitis resistance of said bovine subject and/or off-spring therefrom and assigning a breeding value based on said presence or absence

### FIELD OF INVENTION

The present invention relates to a method for determining resistance to mastitis in a bovine subject comprising detecting at least one genetic marker associated with mastitis resistance. Furthermore, the present invention relates to a kit for detecting the presence or absence of at least one genetic marker associated with resistance to mastitis.

### BACKGROUND OF INVENTION

Mastitis is the inflammation of the mammary gland or udder of the cow resulting from infection or trauma and mastitis is believed to be the most economically important disease in cattle. The disease may be caused by a variety of agents. The primary cause of mastitis is the invasion of the mammary gland via the teat end by microorganisms. Mastitis may be clinical or sub-clinical, with sub-clinical infection preceding clinical manifestations. Clinical mastitis (CM) can be detected visually through observing red and swollen mammary glands i.e. red swollen udder, and through the production of clotted milk. Once detected, the milk from mastitic cows is kept separate from the vat so that it will not affect the overall milk quality. Sub-clinical mastitis is a type of mastitis characterized by high somatic cell counts (SCC), a normal or elevated body temperature, and milk samples that should test positive on culture. Thus, sub-clinical mastitis cannot be detected visually by swelling of the udder or by observation of the gland or the milk produced. Because of this, farmers do not have the option of diverting milk from sub-clinical mastitic cows. However, this milk is of poorer quality than that from non-infected cows and can thus contaminate the rest of the milk in the vat.

Mastitis can be detected by the use of somatic cell counts (SCC) in which a sample of milk from a cow is analysed for the presence of somatic cells (white blood cells). Somatic cells are part of the cow's natural defense mechanism and cell counts rise when the udder becomes infected. The number of somatic cells in a milk sample can be estimated indirectly by rolling-ball viscometer and Coulter counter.

As mastitis results in reduced quantity and quality of milk and products from milk, mastitis results in economic losses to the farmer and dairy industry. Therefore, the ability to determine the genetic basis of resistance to mastitis in a bovine is of immense economic significance to the dairy industry both in terms of daily milk production but also in breeding management, selecting for bovine subjects with resistance to mastitis. A method of genetically selecting bovine subjects with improved resistance that will yield cows less prone to mastitis would be desirable.

Many studies have attempted to detect quantitative trait loci (QTL) affecting mastitis (e.g. Schrooten et al. 2000; Boichard et al. 2003), so that the QTL information could be utilized through marker assisted selection (MAS). Most studies, so far, have identified QTL for somatic cell score (SCS), an indicator trait for clinical mastitis (CM), and not directly for CM. Although these two traits have a high genetic correlation (Lund et al. 1999), it is not known if the QTL that have been identified for SCS also affect CM. It has been shown that persistently high somatic cell count (SCC) levels are mainly a sign of subclinical mastitis which is most often caused by contagious bacteria such as Streptococcus aureus and Streptococcus agalactiae (de Haas et al., 2002). Incidences of acute clinical mastitis are more often caused by environmental bacteria such as Escherichia coli and in these infections the SCC levels increase rapidly but are soon dropping to normal level when the infection is cured. Therefore an acute infection may not be detected by high SCC levels. Another limitation of earlier studies is that the QTL were detected by linkage analysis (LA) with low precision for QTL position and, furthermore, LA associations between markers and the trait can only be used for selection within families. On the contrary, a combined linkage disequilibrium and linkage analysis (LDLA) can potentially fine-map a QTL to a chromosomal region less than 1 cM using closely linked markers (Meuwissen & Goddard 2000). The markers within the LDLA confidence interval can be used to identify haplotypes with predictive ability in the general population. These haplotypes are easier to use in MAS than the LA markers.

Once mapped, a genetic marker can be usefully applied in marker assisted selection. In the present invention genetic markers associated to clinical mastitis and/or SCS have been identified in the bovine genome, which allows for a method for determining whether a bovine subject and its off-spring will be resistant to mastitis.

### SUMMARY OF INVENTION

It is of significant economic interest within the cattle industry to be able to select bovine subjects with increased resistance to mastitis and thereby avoid economic losses in connection with animals suffering from mastitis. The genetic predisposition for resistance to mastitis may be detected by the present invention. The present invention offers a method for determining the resistance to mastitis in a bovine subject based on genetic markers which are associated with and/or linked to resistance to mastitis.

One aspect of the present invention relates to method for determining resistance to mastitis in a bovine subject, comprising detecting in a sample from said bovine subject the presence or absence of at least one genetic marker that is associated with at least one trait indicative of mastitis resistance of said bovine subject and/or off-spring therefrom, wherein said at least one genetic marker is located in a region of the bovine genome selected from the group consisting of regions 1-61 identified in table 2, wherein said regions are delineated by the SNP markers identified in columns 3 and 5, and/or delineated by the genomic position identified in columns 4 and 6.

In another aspect, the present invention relates to a method for selecting a bovine subject for breeding purposes, said method comprising determining resistance to mastitis of said bovine subject and/or off-spring therefrom by a method of the invention, and then selecting or not selecting said bovine subject for breeding based on said determined breeding value.

A third aspect of the present invention relates to a kit for use in detecting the presence or absence in a bovine subject of at least one genetic marker associated with resistance to mastitis, comprising at least one detection member for determining a genetic marker located in a region of the bovine genome selected from the group consisting of regions 1-61 identified in table 2, wherein said regions are delineated by the SNP markers identified in columns 3 and 5, and/or delineated by the genomic position identified in columns 4 and 6.

In a fourth aspect, the invention relates to the use of the kit mentioned above for detecting the presence or absence in a bovine subject of at least one genetic marker associated with resistance to mastitis.

In a fifth aspect, the present invention relates to a method for estimating a breeding value in respect of susceptibility to mastitis in a bovine subject, comprising detecting in a sample from said bovine subject the presence or absence of at least one genetic marker that is associated with at least one trait indicative of mastitis resistance of said bovine subject and/or off-spring therefrom, wherein said at least one genetic marker is located in a region of the bovine genome selected from the group consisting of regions 1-61 of table 2, wherein said regions are delineated by the SNP markers identified in columns 3 and 5, and/or delineated by the genomic position identified in columns 4 and 6.

### DESCRIPTION OF DRAWINGS

FIG. 1. Genome-wide scan for mastitis trait CM (Clinical mastitis all lactations): −log₁₀ of the p-value analysis for association with SNPs. Chromosomes are shown in alternating colors for clarity. The dotted line represents suggestive association [−log 10(p-value)=4] as considered in the present example.

FIG. 2. Genome-wide scan for mastitis trait SCS (Somatic cell score): −log₁₀ of the p-value analysis for association with SNPs. Chromosomes are shown in alternating colors for clarity. The dotted line represents suggestive association [−log 10(p-value)=4] as considered in the present example.

FIG. 3. Genome-wide scan for mastitis trait CM11 (Clinical mastitis first lactation, −15 to 50 days): −log₁₀ of the p-value analysis for association with SNPs. Chromosomes are shown in alternating colors for clarity. The dotted line represents suggestive association [−log 10(p-value)=4] as considered in the present example.

FIG. 4. Genome-wide scan for mastitis trait CM12 (Clinical mastitis first lactation, 51 to 305 days): −log₁₀ of the p-value analysis for association with SNPs. Chromosomes are shown in alternating colors for clarity. The dotted line represents suggestive association [−log 10(p-value)=4] as considered in the present example.

FIG. 5. Genome-wide scan for mastitis trait CM2 (Clinical mastitis second lactation, −15 to 305 days): −log₁₀ of the p-value analysis for association with SNPs. Chromosomes are shown in alternating colors for clarity. The dotted line represents suggestive association [−log 10(p-value)=4] as considered in the present example.

FIG. 6. Genome-wide scan for mastitis trait CM3 (Clinical mastitis third lactation, −15 to 305 days): −log₁₀ of the p-value analysis for association with SNPs. Chromosomes are shown in alternating colors for clarity. The dotted line represents suggestive association [−log 10(p-value)=4] as considered in the present example.

FIG. 7. Manhattan plot for the clinical mastitis between −15 and 50 days after 1st calving (CM11). The X-axis shows the chromosomes and SNPs. The Y-axis shows the −log 10 (p-value) for each SNP which reflects the strength of association for a SNP with the trait analyzed.

FIG. 8. Manhattan plot for clinical mastitis between −51 and 305 days after 1st calving (CM12). The X-axis shows the chromosomes and SNPs. The Y-axis shows the −log 10 (p-value) for each SNP which reflects the strength of association for a SNP with the trait analyzed.

FIG. 9. Manhattan plot for clinical mastitis between −15 and 305 days after 2nd calving (CM2). The X-axis shows the chromosomes and SNPs. The Y-axis shows the −log 10 (p-value) for each SNP which reflects the strength of association for a SNP with the trait analyzed.

FIG. 10. Manhattan plot for clinical mastitis between −15 and 305 days after 3rd calving (CM3). The X-axis shows the chromosomes and SNPs. The Y-axis shows the −log 10 (p-value) for each SNP which reflects the strength of association for a SNP with the trait analyzed.

FIG. 11. Manhattan plot for clinical mastitis index (CM5). The X-axis shows the chromosomes and SNPs. The Y-axis shows the −log 10 (p-value) for each SNP which reflects the strength of association for a SNP with the trait analyzed.

FIG. 12. Manhattan plot for log average somatic cell count in 1st lactation (SCC1). The X-axis shows the chromosomes and SNPs. The Y-axis shows the −log 10 (p-value) for each SNP which reflects the strength of association for a SNP with the trait analyzed.

FIG. 13. Manhattan plot for log average somatic cell count in 2nd lactation (SCC2). The X-axis shows the chromosomes and SNPs. The Y-axis shows the −log 10 (p-value) for each SNP which reflects the strength of association for a SNP with the trait analyzed.

FIG. 14. Manhattan plot for log average somatic cell count in 3rd lactation (SCC3). The X-axis shows the chromosomes and SNPs. The Y-axis shows the −log 10 (p-value) for each SNP which reflects the strength of association for a SNP with the trait analyzed.

FIG. 15. Manhattan plot for log average somatic cell count index (SCC). The X-axis shows the chromosomes and SNPs. The Y-axis shows the −log 10 (p-value) for each SNP which reflects the strength of association for a SNP with the trait analyzed.

FIG. 16: The association of SNP variants identified from whole genome sequence with the first lactation clinical mastitis (CM11) at 88-96 Mb on bovine chromosome 6. The x-axis is the SNP number as order in the bovine genome assembly (UMD3.1) and the y-axis is −log 10(p-values).

FIG. 17. Table 6

FIG. 18. Manhattan plot for BTA5, A. Chr-5.1 MAS11; B. Chr-5.2 MAS12; C. Chr-5.3 MAS2; D. Chr-5.4 MAS3; D. Chr-5.5 MAS-INDEX; F. Chr-5.6 SCS1; G. Chr-5.7 SCS2; H. Chr-5.8 SCS3; I. Chr-5.9 SCS-INDEX

FIG. 19. Manhattan plot for BTA6, A. Chr-6.1 MAS11; B. Chr-6.2 MAS12; C. Chr-6.3 MAS2; D. Chr-6.4 MAS3; D. Chr-6.5 MAS-INDEX; F. Chr-6.6 SCS1; G. Chr-6.7 SCS2; H. Chr-6.8 SCS3; I. Chr-6.9 SCS-INDEX

FIG. 20. Manhattan plot for BTA13, A. Chr-13.1 MAS11; B. Chr-13.2 MAS12; C. Chr-13.3 MAS2; D. Chr-13.4 MAS3; D. Chr-13.5 MAS-INDEX; F. Chr-13.6 SCS1; G. Chr-13.7 SCS2; H. Chr-13.8 SCS3; I. Chr-13.9 SCS-INDEX

FIG. 21. Manhattan plot for BTA16, A. Chr-16.1 MAS11; B. Chr-16.2 MAS12; C. Chr-16.3 MAS2; D. Chr-16.4 MAS3; D. Chr-16.5 MAS-INDEX; F. Chr-16.6 SCS1; G. Chr-16.7 SCS2; H. Chr-16.8 SCS3; I. Chr-16.9 SCS-INDEX

FIG. 22. Manhattan plot for BTA19, A. Chr-19.1 MAS11; B. Chr-19.2 MAS12; C. Chr-19.3 MAS2; D. Chr-19.4 MAS3; D. Chr-19.5 MAS-INDEX; F. Chr-19.6 SCS1; G. Chr-19.7 SCS2; H. Chr-19.8 SCS3; I. Chr-19.9 SCS-INDEX

FIG. 23. Manhattan plot for BTA20, A. Chr-20.1 MAS11; B. Chr-20.2 MAS12; C. Chr-20.3 MAS2; D. Chr-20.4 MAS3; D. Chr-20.5 MAS-INDEX; F. Chr-20.6 SCS1; G. Chr-20.7 SCS2; H. Chr-20.8 SCS3; I. Chr-20.9 SCS-INDEX

FIG. 24. SNP polymorphisms on BTA20 associated with mastitis. The round circles are from the single marker analysis with linear mixed model using the full sequence variants; the black line is the haplotype analysis with 50K genotypes; the green line is the haplotype analysis with 50K including the SNP (rs133218364) located at 33,642,072 Bp on BTA20 as fixed effect in the model; the red line is the haplotype analysis with 50K including the SNP (rs133596506) located at 35,969,994 Bp on BTA20 as fixed effect in the model.

### DETAILED DESCRIPTION OF THE INVENTION

The present invention relates to genetic determinants of mastitis resistance in dairy cattle. The occurrence of mastitis, both clinical and sub-clinical mastitis involves substantial economic loss for the dairy industry. Therefore, it is of economic interest to identity those bovine subjects that have a genetic predisposition for mastitis resistance. Bovine subjects with such genetic predisposition are carriers of desired traits, which can be passed on to their offspring.

### TERMS AND DEFINITIONS

The term “genetic marker” refers to a variable nucleotide sequence (polymorphism) of the DNA on the bovine chromosome and distinguishes one allele from another. The variable nucleotide sequence can be identified by methods known to a person skilled in the art for example by using specific oligonucleotides in for example amplification methods and/or observation of a size difference. However, the variable nucleotide sequence may also be detected by sequencing or for example restriction fragment length polymorphism analysis, or by different hybridization techniques, such as southern blotting or array technologies using oligonucleotide probes. The variable nucleotide sequence may be represented by a deletion, an insertion, repeats, and/or a point mutation.

One type of genetic marker is a microsatellite marker, which may be located in/or coupled to a quantitative trait locus. Microsatellite markers refer to short sequences repeated after each other. In short sequences are for example one nucleotide, such as two nucleotides, for example three nucleotides, such as four nucleotides, for example five nucleotides, such as six nucleotides, for example seven nucleotides, such as eight nucleotides, for example nine nucleotides, such as ten nucleotides. However, changes sometimes occur and the number of repeats may increase or decrease. The specific definition and locus of the polymorphic microsatellite markers can be found in the USDA genetic map (Kappes et al. 1997; or by following the link to U.S. Meat Animal Research Center http://www.marc.usda.gov/genome/cattle/cattle.html). Another type of genetic marker is a single nucleotide polymorphism (SNP). In cattle, it is possible to simultaneously genotype large numbers of SNP markers using the commercially available kits, for example the bovine SNP genotyping kits provided by Illumina Inc.

It is appreciated that the genetic markers of the present invention are genetically linked to traits for mastitis resistance in a bovine subject. However, it is also understood that a number of additional genetic markers may be found in neighbouring DNA regions, and that these markers can be used to infer the identity of genetic markers associated with mastitis provided herein, when such additional genetic markers are genetically coupled to the markers provided by the present invention. Such additional genetic markers are obvious equivalents of the markers provided herein, and such markers are also within the scope of the present invention.

The term ‘Quantitative trait locus (QTL)’ is a region of DNA that is associated with a particular trait (e.g., mastitis resistance, somatic cell count, or clinical mastitis). Though not necessarily genes themselves, QTLs are regions of DNA that are closely linked to the genes that underlie the trait in question.

The term “associated with” as used herein in regards to the genetic marker allele and/or combination of genetic marker alleles and phenotypic traits, is meant to comprise both direct and indirect genetic linkages. Thus, a genetic marker allele and/or combination of genetic marker alleles which are associated with a trait according to the present invention may be coupled to said trait by direct or indirect genetic linkages. Moreover, the term “trait associated with” as used herein in regards to a specific phenotype, relates to any phenotypic traits, which to any extent contribute to said phenotype. For example, the traits somatic cell count (SCC), somatic cell score (SCS), udder conformation (which comprises several quantitative measures, such as fore udder attachment, udder depth, udder texture etc.), and diagnostic variables (such as treated cases of clinical mastitis within a specific timeframe) contribute to the overall mastitis phenotype. Thus, the “traits associated with mastitis resistance”, or “mastitis resistance phenotypic traits” comprise SCC, SCS, CM11, CM12, CM2, CM3, CM, SCC3, SCC2, SCC1, SCC and diagnostic variables, including the subindexes of any of said phenotypic traits.

The term “genetically coupled” is used herein about two genomic loci, which tend to segregate together. Thus, an SNP marker allele, which is genetically coupled to another genetic marker allele associated with a specific phenotypic trait according to the present invention, is indicative of said genetic marker, and may consequently be detected in a sample as an alternative of detecting said genetic marker associated with said phenotypic traits, for example traits associated with mastitis resistance.

It is furthermore appreciated that the nucleotide sequences of the genetic marker allele or combination of marker alleles of the present invention are genetically associated with phenotypic traits of the present invention in a bovine subject. Consequently, it is also understood that a number of genetic markers may be comprised in the nucleotide sequence of the DNA region(s) flanked by and including the genetic markers according to the method of the present invention.

The term “gene” is as used herein is meant to comprise coding regions as well as non-coding region of any genes, as well as upstream and downstream regions of the open reading frame. Thus, a genetic marker “located in a gene” may be located in exons, introns, or upstream or downstream of the open reading frame, for example in the area of 1000 nucleotides or more upstream or downstream of the open reading frame of the gene in question.

Specifically, the transcribed region of a gene is considered to be comprised in the term “gene”, and markers located in a gene, thus, includes any marker located in a transcribed region of that gene.

Linkage Disequilibrium Linkage disequilibrium (LD) reflects recombination events dating back in history and the use of LD mapping within families increases the resolution of mapping. LD exists when observed haplotypes in a population do not agree with the haplotype frequencies predicted by multiplying together the frequency of individual genetic markers in each haplotype. In this respect the term haplotype means a set of closely linked genetic markers present on one chromosome which tend to be inherited together. In order for LD mapping to be efficient the density of genetic markers needs to be compatible with the distance across which LD extends in the given population. Linkage disequilibrium reflects the extent to which different genetic markers tend to be co-inherited in a population. In cattle the level of LD is high compared to for example human, due to i.a. inbreeding and historical bottlenecks. Therefore, the identity of one genetic marker can often be inferred from the identity of alternative genetic markers, which are in LD.

### Granddaughter Design

The granddaughter design includes analysing data from DNA-based markers for grand sires that have been used extensively in breeding and for sons of grand sires where the sons have produced offspring. The phenotypic data that are to be used together with the DNA-marker data are derived from the daughters of the sons. Such phenotypic data could be for example milk production features, features relating to calving, meat quality, or disease. One group of daughters have inherited one allele from their father whereas a second group of daughters have inherited the other allele form their father. By comparing data from the two groups information can be gained whether a fragment of a particular chromosome is harbouring one or more genes that affect the trait in question. It may be concluded whether a QTL is present within this fragment of the chromosome. A prerequisite for performing a granddaughter design is the availability of detailed phenotypic data. In the present invention such data have been available (http://www.lr.dk/kvaeg/diverse/principles.pdf). Genes conferring quantitative traits to an individual may be found in an indirect manner by observing pieces of chromosomes that act as if one or more gene(s) is located within that piece of the chromosome. In contrast, DNA markers can be used directly to provide information of the traits passed on from parents to one or more of their off spring when a number of DNA markers on a chromosome have been determined for one or both parents and their off-spring. The markers may be used to calculate the genetic history of the chromosome linked to the DNA markers.

### Bovine Subject

The term “bovine subject” refers to cattle of any breed and is meant to include both cows and bulls, whether adult or newborn animals. No particular age of the animals are denoted by this term. One example of a bovine subject is a member of the Holstein breed. In one preferred embodiment, the bovine subject is a member of the Holstein-Friesian cattle population. In one embodiment, the bovine subject is a member of the Danish and/or Swedish Holstein cattle population. In another embodiment, the bovine subject is a member of the Holstein Swartbont cattle population. In another embodiment, the bovine subject is a member of the Deutsche Holstein Schwarzbunt cattle population. In another embodiment, the bovine subject is a member of the US Holstein cattle population. In one embodiment, the bovine subject is a member of the Red and White Holstein breed. In another embodiment, the bovine subject is a member of the Deutsche Holstein Schwarzbunt cattle population.

In one embodiment, the bovine subject is a member of any family, which include members of the Holstein breed. In one preferred embodiment the bovine subject is a member of the Danish Red population. In another preferred embodiment the bovine subject is a member of the Finnish Ayrshire population. In yet another embodiment the bovine subject is a member of the Swedish Red and White population. In a further embodiment the bovine subject is a member of the Danish Holstein population. In another embodiment, the bovine subject is a member of the Swedish Red and White population. In yet another embodiment, the bovine subject is a member of the Nordic Red population. In yet another embodiment, the bovine subject is a member Nordic Holstein, Danish Jersey and Nordic Red breed

In one embodiment of the present invention, the bovine subject is selected from the group consisting of Swedish Red and White, Danish Red, Finnish Ayrshire, Holstein-Friesian, Danish Holstein and Nordic Red. In another embodiment of the present invention, the bovine subject is selected from the group consisting of Finnish Ayrshire and Swedish Red and White cattle. In another embodiment of the present invention, the bovine subject is selected from the group consisting of Finnish Ayrshire and Swedish Red and White cattle.

### Mastitis Resistance

The term “mastitis” relates to the inflammation of the mammary gland of the udder of a cow. In the present application the term “mastitis” is used to describe both clinical mastitis and sub-clinical mastitis, which can be characterized for example by high somatic cell score (SCS).

The terms “mastitis resistance” and ‘resistance to mastitis’ are used interchangeable and relates to the fact that some bovine subjects are not as prone to mastitis as are other bovine subjects, in other words, some bovine subjects are less susceptible to mastitis than other bovine subjects. Thus, the term “resistance” as used herein, refers to any level of reduction in mastitis, ranging from a minute reduction of 0.5% or less to complete absence of mastitis, i.e. complete resistance. When performing analyses of a number of bovine subjects as in the present invention in order to determine genetic markers that are associated with resistance to mastitis, the traits implying resistance to mastitis may be observed by the presence or absence of genetic markers linked to occurrence of clinical mastitis and/or sub-clinical mastitis in the bovine subjects analyzed. It is understood that mastitis resistance comprise resistance to traits, which affect udder health in the bovine subject or its off-spring. Thus, mastitis resistance of a bull is physically manifested by its female off-spring.

Mastitis resistance is inversely correlated with susceptibility to mastitis, i.e. a bovine subject with high mastitis resistance has low susceptibility to mastitis. Thus, the term “susceptible to mastitis” as used herein is meant to indicate that a bovine subject has a relatively higher likelihood of suffering from mastitis, or having a trait indicative of mastitis.

### Traits Indicative of Mastitis Resistance

Daughters of bulls can be scored for mastitis resistance on the basis of a number of different quantitative and qualitative parameters. Specifically, mastitis resistance may be observed according to the present invention on the basis of specific traits, which are indicative of mastitis resistance. One such trait indicative of mastitis resistance in a population of cattle is recorded cases of clinical mastitis. Other examples of traits are somatic cell count (SCC), or somatic cell score (SCS), which is defined as the mean of log¹⁰ transformed somatic cell count values (in 10,000/mL) obtained from the milk recording scheme. The mean is for example taken over the period 10 to 180 days after calving. Estimated breeding values (EBV) for traits of sons may be calculated using a single trait Best Linear Unbiased Prediction (BLUP) animal model ignoring family structure. Examples of specific quantitative traits indicative of mastitis resistance are provided in the table below:

TABLE 1
Definitions of exemplary traits associated with
mastitis according to the present invention.

| Trait | Trait | |
|---|---|---|
| No. | abbreviation | Trait definitions |
| 1 | CM11 | Clinical mastitis (1) or not (0) between −15 and |
| | | 50 days after 1st calving |
| 2 | CM12 | Clinical mastitis (1) or not (0) between 51 and |
| | | 305 days after 1st calving |
| 3 | CM2 | Clinical mastitis (1) or not (0) between −15 and |
| | | 305 days after 2nd calving |
| 4 | CM3 | Clinical mastitis (1) or not (0) between −15 and |
| | | 305 days after 3rd calving |
| 5 | CM | Clinical mastitis: 0.25*CM11 + 0.25*CM12 + |
| | | 0.3*CM2 + 0.2*CM3 |
| 6 | SCC1 | Log. somatic cell count average in 1st lactation |
| 7 | SCC2 | Log. somatic cell count average in 2nd lactation |
| 8 | SCC3 | Log. somatic cell count average in 3rd lactation |
| 9 | SCC | Log somatic cell count: 0.5*SCC1 + 0.3*SCC2 + |
| | | 0.2*SCC3 |

In one embodiment of the present invention, the methods and kits described herein relates to mastitis resistance, such as resistance to clinical mastitis and/or resistance to sub-clinical mastitis, such as detected by somatic cell counts or SCS. More specifically, the methods and kits of the invention relates in one embodiment to genetic markers associated with at least one trait indicative of mastitis, such trait in a preferred embodiment being selected from CM11 (Clinical mastitis (1) or not (0) between −15 and 50 days after 1st calving), CM12 (Clinical mastitis (1) or not (0) between 51 and 305 days after 1st calving), CM2 (Clinical mastitis (1) or not (0) between −15 and 305 days after 2nd calving), CM3 (Clinical mastitis (1) or not (0) between −15 and 305 days after 3rd calving), CM (Clinical mastitis: 0.25*CM11+0.25*CM12+0.3*CM2+0.2*CM3), SCC1 (Log. somatic cell count average in 1st lactation), SCC2 (Log. somatic cell count average in 2nd lactation), SCC3 (Log. somatic cell count average in 3rd lactation) and SCC (Log somatic cell count: 0.5*SCC1+0.3*SCC2+0.2*SCC3). In a preferred embodiment, the trait is clinical mastitis, for example any trait selected from CM11, CM12, CM2, CM3 or CM. As specified in table 1, CM is an index for clinical mastitis based on CM11, CM12, CM2 and CM3.

In yet another embodiment, the method and kit of the present invention primarily relates to resistance to clinical mastitis in combination with resistance to sub-clinical mastitis such as detected by somatic cell counts or SCS, for example SCC1, SCC2, SCC3 or SCC. The methods and kits of the present invention comprise detecting the presence or absence of at least one genetic marker that is associated with at least one trait indicative of mastitis resistance of a bovine subject or off-spring therefrom, wherein said at least one trait is selected from somatic cell count (SCC), somatic cell score (SCS) and/or clinical mastitis.

In general, increased levels of SCS are indicative of mastitis, e.g. subclinical mastitis. The level of SCC may be increased compared to previous measures for the same bovine subject, or compared to an average SCC for the given population, breed, or family. The SCS level may be measured at any time, and may be separate measures or a mean value over one lactation period. For example, an SCC level above 100.000 cells/ml milk, such as above 200.000, for example above 300.000 cells/ml milk, such as above 400.000, for example above 500.000 cells/ml milk, such as above 600.000, cell/ml milk is indicative of mastitis, such as clinical or subclinical mastitis. Therefore, SCC levels of such magnitudes are considered as traits indicative of reduced susceptibility to mastitis according to the present invention. However, the level of SCC indicative of mastitis resistance or susceptibility to mastitis may vary for different bovine subjects, breeds and families.

The present invention can be used to estimate breeding values in respect of mastitis resistance or susceptibility to mastitis. True breeding value is the genetic merit of an individual which can be conceptually defined as twice the average deviation of its offspring from the population mean when mated randomly to an infinite population. It is an estimate of the ability of an individual to produce superior offspring. True breeding values are not known but can be estimated from the animals own performance and/or the performance of its offspring and/or other relatives. In addition to, or instead of, phenotypic performance, information about animals genotypes at certain genes or markers associated with the trait of interest can be used in breeding value estimation procedures. Use of such information can increase the reliability of the breeding values and make, for example, selection possible at a younger age. In one embodiment, the at least on genetic marker indicative of mastitis resistance is used to estimate the breeding value of a bovine subject.

The trait indicative of mastitis resistance may be recalculated into a breeding value for every bovine subject, for example every sire. Thus, the genetic markers of the methods and kits of the present invention may be used for selection of bovine subjects with increased breeding values, and detection of at least on genetic marker indicative of mastitis resistance according to the present invention is indicative of an increased breeding value of the bovine subject. For example the breeding value is increased by at least 0.5%, such as at least 1%, such as at least 2, 3, 4, 5, 6, 7, 8, 9, for example at least 10%.

### Sample

The method according to the present invention includes analyzing a sample of a bovine subject, wherein said sample may be any suitable sample capable of providing the bovine genetic material for use in the method. The type of sample is not important, as long as the sample comprise genetic material specific for the bovine subject, which is analysed. Thus, any sample comprising genetic material from the bovine subject can be used. Preferably, the sample is a sample, which is easily obtained from the bovine subject, preferably a sample, which can be obtained without any invasive procedures.

Thus, mastitis resistance is determined by detecting the absence or presence of a genetic marker allele in a sample of any source comprising genetic material. The bovine genetic material may for example be extracted, isolated and/or purified if necessary. The samples may be fresh or frozen. Detection of a genetic marker may be performed on samples selected from the group consisting of blood, semen (sperm), urine, liver tissue, muscle, skin, hair, follicles, ear, tail, fat, testicular tissue, lung tissue, saliva, spinal cord biopsy and/or any other tissue.

In preferred embodiments the sample is selected from the group consisting of semen (sperm), blood, urine, skin, hair, ear, tail, and muscle. In another preferred embodiment the sample is selected from the group consisting of blood. In particularly preferred embodiments the sample is milk. In another particularly preferred embodiment the sample is skin tissue. In yet another particularly preferred embodiment the sample is muscle. In a most preferred embodiment the sample is semen (sperm).

For microsatellite or SNP genotyping, nucleic acid may be extracted from the samples by a variety of techniques. For example Genomic DNA may be isolated from the sample by treatment with proteinase K followed by extraction with phenol (see e.g. Sambrook et al. 1989). However, the sample may also be used directly.

The amount of the nucleic acid used for microsatellite or SNP genotyping for detection of a genetic marker according to the method of the present invention is in the range of nanograms to micrograms. It is appreciated by the person skilled in the art that in practical terms no upper limit for the amount of nucleic acid to be analysed exists. The problem that the skilled person encounters is that the amount of sample to be analysed is limited. Therefore, it is beneficial that the method of the present invention can be performed on a small amount of sample and thus a limited amount of nucleic acid in the sample is required. The amount of the nucleic acid to be analysed is thus at least 1 ng, such as at least 10 ng, for example at least 25 ng, such as at least 50 ng, for example at least 75 ng, such as at least 100 ng, for example at least 125 ng, such as at least 150 ng, for example at least 200 ng, such as at least 225 ng, for example at least 250 ng, such as at least 275 ng, for example at least 300 ng, 400 ng, for example at least 500 ng, such as at least 600 ng, for example at least 700 ng, such as at least 800, ng, for example at least 900 ng or such as at least 1000 ng.

In one preferred embodiment the amount of nucleic acid as the starting material for the method of the present invention is 20-50 ng. In a specifically preferred embodiment, the starting material for the method of the present invention is at 30-40 ng.

### Chromosomal Regions and Markers

BTA is short for Bos taurus autosome.

One aspect of the present invention relates to a method for determining resistance to mastitis in a bovine subject, comprising detecting in a sample from said bovine subject the presence or absence of at least one genetic marker that is associated with at least one trait indicative of mastitis resistance of said bovine subject and/or off-spring therefrom, wherein said at least one genetic marker is located in a genetic region of the bovine genome selected from region 1-61, as specified in table 2.

TABLE 2

| 1 | | 3 | | 5 | | | 10 |
|---|---|---|---|---|---|---|---|
| Region | 2 | Start- | 4 | End- | 6 | 9 | Top SNP |
| No. | Chr | SNP | Start Pos. | SNP | End Pos. | Most sig. SNP name | Pos |
| 1 | 1 | 19479 | 76096755 | 19481 | 76099500 | Bo- | 76096755 |
| | | | | | | vineHD0100021877 | |
| 2 | 1 | 24128 | 96507612 | 24500 | 97612639 | Bo- | 96507612 |
| | | | | | | vineHD0100027421 | |
| 3 | 1 | 33740 | 135236190 | 35634 | 141791717 | Bo- | 135285949 |
| | | | | | | vineHD0100038448 | |
| 4 | 3 | 16606 | 62218619 | 16846 | 63185254 | ARS-BFGL-NGS- | 62615411 |
| | | | | | | 57708 | |
| 5 | 3 | 23488 | 92199528 | 25665 | 101364920 | Bo- | 101323866 |
| | | | | | | vineHD0300028997 | |
| 6 | 4 | 5485 | 20993524 | 7036 | 27829152 | Bo- | 27829152 |
| | | | | | | vineHD0400008053 | |
| 7 | 4 | 8924 | 36558317 | 10527 | 44073697 | Hapmap24419-BTA- | 36558317 |
| | | | | | | 162106 | |
| 8 | 4 | 13365 | 55763368 | 15735 | 65519029 | Bo- | 61125903 |
| | | | | | | vineHD0400016706 | |
| 9 | 4 | 23730 | 97674762 | 24213 | 99540028 | Bo- | 99540028 |
| | | | | | | vineHD0400027868 | |
| 10 | 5 | 16168 | 67417898 | 17489 | 72243381 | ARS-BFGL-NGS- | 72243381 |
| | | | | | | 70198 | |
| 11 | 5 | 20435 | 84539347 | 27159 | 109948232 | Bo- | 86998734 |
| | | | | | | vineHD0500024659 | |
| 12 | 6 | 4475 | 18036724 | 7462 | 29334848 | Bo- | 23549700 |
| | | | | | | vineHD0600006497 | |
| 13 | 6 | 13573 | 51683927 | 13598 | 51755112 | Bo- | 51731374 |
| | | | | | | vineHD0600014264 | |
| 14 | 6 | 18708 | 71082832 | 26792 | 102757841 | Bo- | 88919352 |
| | | | | | | vineHD0600024355 | |
| 15 | 7 | 1236 | 5202111 | 1708 | 6663939 | Bo- | 5927298 |
| | | | | | | vineHD0700001692 | |
| 16 | 7 | 2907 | 14485587 | 4789 | 22681472 | Bo- | 18032163 |
| | | | | | | vineHD0700005054 | |
| 17 | 7 | 7174 | 31432538 | 10157 | 41607314 | Bo- | 33485418 |
| | | | | | | vineHD4100005904 | |
| 18 | 7 | 10795 | 44074131 | 15561 | 63839308 | Bo- | 63839308 |
| | | | | | | vineHD0700018462 | |
| 19 | 7 | 26534 | 104753300 | 27857 | 109584677 | Bo- | 109406393 |
| | | | | | | vineHD0700031919 | |
| 20 | 8 | 801 | 3101470 | 1541 | 5993074 | Bo- | 4844864 |
| | | | | | | vineHD0800001554 | |
| 21 | 8 | 4831 | 20417406 | 8352 | 35930652 | Bo- | 22287380 |
| | | | | | | vineHD0800006734 | |
| 22 | 9 | 1495 | 7453669 | 1591 | 7749361 | Bo- | 7735822 |
| | | | | | | vineHD0900001741 | |
| 23 | 9 | 2848 | 12242079 | 3079 | 13035215 | Bo- | 12963863 |
| | | | | | | vineHD0900003387 | |
| 24 | 9 | 21143 | 86380558 | 21144 | 86381215 | Bo- | 86380558 |
| | | | | | | vineHD0900024208 | |
| 25 | 10 | 12689 | 47838479 | 13661 | 51407940 | Bo- | 49359005 |
| | | | | | | vineHD1000014875 | |
| 26 | 10 | 15921 | 62168320 | 20229 | 79735238 | Bo- | 74285470 |
| | | | | | | vineHD1000021167 | |
| 27 | 10 | 22654 | 89224445 | 24333 | 94083525 | BTA-80363-no-rs | 90484606 |
| 28 | 11 | 68 | 210963 | 1555 | 4567617 | Bo- | 210963 |
| | | | | | | vineHD4100008447 | |
| 29 | 11 | 23860 | 88133102 | 24010 | 88778399 | Bo- | 88778399 |
| | | | | | | vineHD1100025584 | |
| 30 | 12 | 787 | 2569573 | 933 | 2991581 | Bo- | 2917822 |
| | | | | | | vineHD1200000926 | |
| 31 | 12 | 3217 | 11578657 | 7626 | 27097379 | Bo- | 22865273 |
| | | | | | | vineHD1200006858 | |
| 32 | 12 | 11277 | 44331491 | 11285 | 44349649 | Bo- | 44331491 |
| | | | | | | vineHD1200012284 | |
| 33 | 12 | 15918 | 62561736 | 17398 | 68494212 | Bo- | 63068164 |
| | | | | | | vineHD1200017277 | |
| 34 | 13 | 11798 | 53471793 | 15089 | 70173150 | Bo- | 59588546 |
| | | | | | | vineHD1300017074 | |
| 35 | 14 | 3505 | 13282075 | 5041 | 20691077 | Bo- | 20662703 |
| | | | | | | vineHD1400005926 | |
| 36 | 14 | 9864 | 43961811 | 15451 | 69623868 | Bo- | 51548605 |
| | | | | | | vineHD1400014643 | |
| 37 | 15 | 2329 | 9897946 | 2334 | 9915788 | Bo- | 9897946 |
| | | | | | | vineHD1500002610 | |
| 38 | 15 | 6316 | 26178933 | 8176 | 33293128 | Bo- | 31105101 |
| | | | | | | vineHD1500008366 | |
| 39 | 15 | 9855 | 39284002 | 13327 | 52111223 | Bo- | 43914509 |
| | | | | | | vineHD1500012201 | |
| 40 | 15 | 17079 | 66540919 | 17084 | 66551171 | Bo- | 66543720 |
| | | | | | | vineHD1500019116 | |
| 41 | 16 | 1694 | 8171169 | 2172 | 10545502 | Bo- | 8171169 |
| | | | | | | vineHD1600002326 | |
| 42 | 16 | 3534 | 15737429 | 3596 | 16009799 | Bo- | 15784091 |
| | | | | | | vineHD1600004272 | |
| 43 | 16 | 5299 | 21799660 | 16175 | 64955150 | Bo- | 52924145 |
| | | | | | | vineHD1600014622 | |
| 44 | 17 | 512 | 2467836 | 3752 | 13800376 | Bo- | 9472006 |
| | | | | | | vineHD1700002674 | |
| 45 | 17 | 16389 | 61406860 | 16431 | 61535420 | ARS-BFGL-NGS- | 61522805 |
| | | | | | | 26121 | |
| 46 | 18 | 6383 | 21603442 | 6944 | 23535823 | Bo- | 21606994 |
| | | | | | | vineHD1800006666 | |
| 47 | 18 | 11892 | 41653211 | 13902 | 48570545 | Bo- | 44778431 |
| | | | | | | vineHD1800013234 | |
| 48 | 19 | 2020 | 8230088 | 3676 | 14585690 | Bo- | 14578566 |
| | | | | | | vineHD1900003860 | |
| 49 | 19 | 7734 | 27998517 | 8035 | 29383514 | Bo- | 29320178 |
| | | | | | | vineHD1900008608 | |
| 50 | 19 | 9209 | 33351947 | 12120 | 46467474 | Bo- | 43098630 |
| | | | | | | vineHD1900012270 | |
| 51 | 19 | 12750 | 49013784 | 16762 | 62339802 | Bo- | 55615219 |
| | | | | | | vineHD1900015719 | |
| 52 | 20 | 5111 | 18072225 | 5122 | 18110885 | Bo- | 18110885 |
| | | | | | | vineHD2000005443 | |
| 53 | 20 | 7852 | 28291423 | 14407 | 55744850 | Bo- | 35981673 |
| | | | | | | vineHD2000010279 | |
| 54 | 20 | 14681 | 56557595 | 19739 | 71359405 | Bo- | 67376802 |
| | | | | | | vineHD2000019538 | |
| 55 | 21 | 11020 | 43772475 | 11021 | 43773986 | Bo- | 43772475 |
| | | | | | | vineHD2100012534 | |
| 56 | 22 | 6727 | 24494154 | 8368 | 31397754 | Hapmap38325-BTA- | 25113789 |
| | | | | | | 53915 | |
| 57 | 23 | 1077 | 4758944 | 3549 | 14524909 | Bo- | 11512182 |
| | | | | | | vineHD2300002833 | |
| 58 | 23 | 4429 | 18006108 | 7776 | 28819118 | Bo- | 26369699 |
| | | | | | | vineHD2300007202 | |
| 59 | 23 | 9221 | 33362170 | 9673 | 35604326 | Bo- | 34251317 |
| | | | | | | vineHD2300010058 | |
| 60 | 23 | 11058 | 41491498 | 13747 | 51051152 | Bo- | 44312928 |
| | | | | | | vineHD2300012843 | |
| 61 | 25 | 3879 | 12927936 | 3879 | 12927936 | Bo- | 12927936 |
| | | | | | | vineHD2500003616 | |

In one embodiment, the genetic marker of the invention is selected from the group of markers set forth in table 2, column 9 or 10.

In another embodiment, the genetic marker is selected from the group consisting of the SNPs set forth in tables 10, 12, 13, 15, 16, 18, 19, 21, 23 and 24, cf. the examples herein below.

In another embodiment, the genetic marker is located in a gene selected from the group consisting of the genes set forth in tables 11, 14, 17, 20, 22 and 25, cf. the examples herein below.

In another embodiment, the genetic markers is selected from the group consisting of ss86284888, rs41649041, ss61565956, ss86341106, ss86317725, ss86328358, rs41812941, ss86327354, and rs41940571 (cf. table 3).

In another embodiment, the genetic markers is selected from the group consisting of ss86328743, rs41618669, ss86284888, rs41580905, rs41649041, rs43706944, rs42189699, rs42553026, rs41664497, rs41664497, ss86290235, ss86340493, ss86305923, ss86330005, ss86340725, rs29015635, rs42895750, ss117968104, rs29017739, rs29001782, rs41588957, ss86307579, ss86317213, rs41610991, ss117968170, ss117968764, ss117968030, ss117968525, rs29019575, ss117968738, ss86326721, ss86341106, ss86341106, rs29010419, rs29022799, ss86278591, ss86337596, rs43338539, ss86296213, rs42766480, rs41617692, ss117963883, rs43475842, rs29019286, ss86292503, ss86317725, ss86290731, ss86332750, ss86335834, ss86340346, ss105239139, ss117971362, ss86287919, ss86329615, ss86301882, ss86328358, ss117971370, ss117971325, ss86339873, ss117971671, ss117971176, rs41807595, rs41807595, rs29023167, ss86303613, ss86283374, ss86328473, ss86307986, rs41603818, rs41812941, ss105262977, ss105262977, rs42465037, ss86327354, ss86327432, ss61484557, rs42329877, ss86333005, ss86306906, ss117972835, rs41938511, rs42542144, rs41940571, rs41947330, rs29018751, rs41581087, ss105263178, rs41641052, rs41641055, ss86292111, rs41600165 and ss86306865 (cf. table 4).

Due to linkage disequilibrium as described herein, the present invention also relates to methods for determining the resistance to mastitis in a bovine subject, wherein the at least one genetic marker is linked or genetically coupled to genetic determinants of a bovine trait for resistance to mastitis. In order to determine resistance to mastitis in a bovine subject, it is appreciated that more than one genetic marker may be employed in the present invention. For example the at least one genetic marker may be a combination of at least two or more genetic markers such that the accuracy may be increased, such as at least three genetic markers, for example four genetic markers, such as at least five genetic markers, for example six genetic markers, such as at least seven genetic markers, for example eight genetic markers, such as at least nine genetic markers, for example ten genetic markers.

The at least one genetic marker may be located on at least one bovine chromosome, such as two chromosomes, for example three chromosomes, such as four chromosomes, for example five chromosomes, and/or such as six chromosomes. Thus, the at least one genetic marker may be a combination of markers located on different chromosomes. The at least one genetic marker is selected from any of the individual markers of the tables shown herein below.

In one embodiment of the invention the at least one genetic marker is located on the bovine chromosome BTA1 in a region delineated by BovineHD Genotyping BeadChip SNP#19479 and SNP#19481 and/or in a region between base nos. 76096755 and 76099500, for example, the marker is BovineHD0100021877 or is BovineHD Genotyping BeadChip SNP#76096755, or is linked to any of said markers

In another embodiment of the invention the at least one genetic marker is located on the bovine chromosome BTA3 in a region delineated by BovineHD Genotyping BeadChip SNP#23488 and SNP#25665 and/or in a region between base nos. 92199528 and 101364920, for example, the marker is BovineHD0300028997 or is BovineHD Genotyping BeadChip SNP#101323866, or is linked to any of said markers.

### BTA5

In another embodiment of the invention the at least one genetic marker is located on the bovine chromosome BTA5 in a region delineated by BovineHD Genotyping BeadChip SNP#20435 and SNP#27159 and/or in a region between base nos. 84539347 and 109948232, for example, the marker is BovineHD0500024659 or is BovineHD Genotyping BeadChip SNP#86998734, or is linked to any of said markers.

In one embodiment, the genetic marker is located on the bovine chromosome BTA5 in a region between 84-95 Mb, for example the marker is Chr5_{—}92753829 and/or the trait is mastitis resistance, such as CM11. In one embodiment, the genetic marker is selected from the group consisting of Chr5_{—}92753829, BovineHD0500024659, Chr5_{—}87360522, BovineHD0500026657, Chr5_{—}92753829, Chr5_{—}87360522, Chr5_{—}94040670, Chr5_{—}89528205 and Chr5_{—}87360522 (cf. table 10), and/or the genetic marker allele associated with increased mastitis resistance, and/or the specific trait is as indicated in table 10.

In one embodiment, the genetic marker is located in a gene selected from the group consisting of ENSBTAG00000022360, ENSBTAG00000005833, ENSBTAG00000001673, ENSBTAG00000013202, ENSBTAG00000047048, ENSBTAG00000046178, ENSBTAG00000020715, ENSBTAG00000030493, ENSBTAG00000013541, ENSBTAG00000008541 and ENSBTAG00000009444, cf. table 11.

### BTA6

In another embodiment of the invention the at least one genetic marker is located on the bovine chromosome BTA6 in a region delineated by BovineHD Genotyping BeadChip SNP#18708 and SNP#26792 and/or in a region between base nos. 71082832 and 102757841, for example, the marker is BovineHD0600024355 or is BovineHD Genotyping BeadChip SNP#88919352, or is linked to any of said markers.

However, in a particularly preferred embodiment, the at least one genetic marker is located on the bovine chromosome BTA6 in a region between base nos. 88000560 and 95999980. In a specifically preferred embodiment, the at least one genetic marker is BovineHD0600024355 located at 88,919,352 Bp on BTA6. In one embodiment, BovineHD0600024355 is a genetic marker associated with clinical mastitis, such as CM11.

For example, the at least one genetic marker is located in the region between base nos. 89,052,210 and 89,059,348 on BTA6. Thus, in one preferred embodiment, the genetic marker associated with at least one trait indicative of mastitis, such as clinical mastitis, for example CM11, is located in the neuropeptide FF receptor 2 (NPFFR2) gene, in particular in the coding region of NPFFR2. In one embodiment, the genetic marker associated with mastitis is the chr6_{—}89059253 SNP, which is located at 89,059,253 Bp on BTA6. This SNP is a G-A substitution. However, as alternative SNPs located within the NPFFR2 gene are strongly coupled to the chr6_{—}89059253 SNP, any genetic marker polymorphism located in the NPFFR2 gene is associated with a trait indicative of mastitis. Thus, the present invention relates to methods of determining mastitis and/or a breeding value as well as methods for selected cattle for breeding, and kits, wherein the at least one genetic marker is located in the NPFFR2 gene or is genetically coupled to the NPFFR2 gene, and in one preferred embodiment, the at least one genetic marker is the chr6_{—}89059253 SNP and/or any genetic marker polymorphism genetically coupled thereto. Thus, in one embodiment, the genetic marker is the G/A SNP located at 89,059,253 Bp (UMD3.1), wherein the A allele is associated with mastitis and the G allele is associated with resistance to mastitis.

In one embodiment, the genetic marker is located on the bovine chromosome BTA6 in a region between 88-96 Mb, for example the marker is Chr6_{—}88977023 and/or the trait is mastitis resistance, such as CM11. In one embodiment, the genetic marker is selected from the group consisting of Chr6_{—}88977023, Chr6_{—}88612186, Chr6_{—}88610743, Chr6_{—}88977023, Chr6_{—}88977023, Chr6_{—}88326504, Chr6_{—}88326504, Chr6_{—}88326504 and Chr6_{—}88326504 (cf. table 12), and/or the genetic marker allele associated with increased mastitis resistance, and/or the specific trait is as indicated in table 12. In one embodiment, the marker is Chr6_{—}89059253 and the allele associated with mastitis resistance is the G-allele.

In one embodiment, the genetic marker is located in a gene selected from the group consisting of ENSBTAG00000018531, ENSBTAG00000009310, ENSBTAG00000016795, ENSBTAG00000008577, ENSBTAG00000016290, ENSBTAG00000012397, ENSBTAG00000002348, ENSBTAG00000013718, ENSBTAG00000009070 and ENSBTAG00000006507, cf. table 14.

### BTA7

In another embodiment of the invention the at least one genetic marker is located on the bovine chromosome BTA7 in a region delineated by BovineHD Genotyping BeadChip SNP#2907 and SNP#4789 and/or in a region between base nos. 14485587 and 22681472, for example, the marker is BovineHD0700005054 or is BovineHD Genotyping BeadChip SNP#18032163, or is linked to any of said markers.

In another embodiment of the invention the at least one genetic marker is located on the bovine chromosome BTA7 in a region delineated by BovineHD Genotyping BeadChip SNP#7174 and SNP#10157 and/or in a region between base nos. 31432538 and 41607314, for example, the marker is BovineHD4100005904 or is BovineHD Genotyping BeadChip SNP#33485418, or is linked to any of said markers.

### BTA12

In another embodiment of the invention the at least one genetic marker is located on the bovine chromosome BTA12 in a region delineated by BovineHD Genotyping BeadChip SNP#787 and SNP#933 and/or in a region between base nos. 2569573 and 2991581, for example, the marker is BovineHD1200000926 or is BovineHD Genotyping BeadChip SNP#2917822, or is linked to any of said markers.

In another embodiment of the invention the at least one genetic marker is located on the bovine chromosome BTA12 in a region delineated by BovineHD Genotyping BeadChip SNP#3217 and SNP#7626 and/or in a region between base nos. 11578657 and 27097379, for example, the marker is BovineHD1200006858 or is BovineHD Genotyping BeadChip SNP#22865273, or is linked to any of said markers.

In another embodiment of the invention the at least one genetic marker is located on the bovine chromosome BTA12 in a region delineated by BovineHD Genotyping BeadChip SNP#15918 and SNP#17398 and/or in a region between base nos. 62561736 and 68494212, for example, the marker is BovineHD1200017277 or is BovineHD Genotyping BeadChip SNP#63068164, or is linked to any of said markers.

### BTA13

In another embodiment of the invention the at least one genetic marker is located on the bovine chromosome BTA13 in a region delineated by BovineHD Genotyping BeadChip SNP#11798 and SNP#15089 and/or in a region between base nos. 53471793 and 70173150, for example, the marker is BovineHD1300017074 or is BovineHD Genotyping BeadChip SNP#59588546, or is linked to any of said markers.

In one embodiment, the genetic marker is located on the bovine chromosome BTA13 in a region between 57-63 Mb, for example the marker is Chr13_{—}57608628 and/or the trait is mastitis resistance, such as CM. In one embodiment, the genetic marker is selected from the group consisting of Chr13_{—}57608336, Chr13_{—}57608354, Chr13_{—}59584651, Chr13_{—}59584651, Chr13_{—}57608628, Chr13_{—}57608354, Chr13_{—}60621602, Chr13_{—}60621602 and Chr13_{—}60621602 (cf. table 15), and/or the genetic marker allele associated with increased mastitis resistance, and/or the specific trait is as indicated in table 15. In one embodiment, the marker is Chr13_{—}57579568 and the allele associated with mastitis resistance is the T-allele, and/or the marker is Chr13_{—}57579569 and the allele associated with mastitis resistance is the G-allele.

In one embodiment, the genetic marker is located in a gene selected from the group consisting of ENSBTAG00000020261, ENSBTAG00000012109, ENSBTAG00000018053, ENSBTAG00000018418, ENSBTAG00000013330, ENSBTAG00000048288, ENSBTAG00000003364, ENSBTAG00000048009, ENSBTAG00000027384, ENSBTAG00000027383, ENSBTAG00000020555, ENSBTAG00000031254, ENSBTAG00000016169, ENSBTAG00000016348, ENSBTAG00000019200, ENSBTAG00000010112, ENSBTAG00000038687 and ENSBTAG00000038412, cf. table 17.

### BTA16

In another embodiment of the invention the at least one genetic marker is located on the bovine chromosome BTA16 in a region delineated by BovineHD Genotyping BeadChip SNP#5299 and SNP#16175 and/or in a region between base nos. 21799660 and 64955150, for example, the marker is BovineHD1600014622 or is BovineHD Genotyping BeadChip SNP#52924145, or is linked to any of said markers.

In one embodiment, the genetic marker is located on the bovine chromosome BTA16 in a region between 48-55 Mb, for example the marker is Chr16_{—}50529178 and/or the trait is mastitis resistance, such as CM11. In one embodiment, the genetic marker is selected from the group consisting of Chr16_{—}50529178, Chr16_{—}49054912, Chr16_{—}49054912, Chr16_{—}54246279, Chr16_{—}50532600, Chr16_{—}52097973, Chr16_{—}53806663, Chr16_{—}53806663 and Chr16_{—}53998150 (cf. table 18), and/or the genetic marker allele associated with increased mastitis resistance, and/or the specific trait is as indicated in table 18. In one embodiment, the marker is Chr16_{—}50529178 and the allele associated with mastitis resistance is the A-allele, and/or the marker is Chr16_{—}50564280 and the allele associated with mastitis resistance is the T-allele.

In one embodiment, the genetic marker is located in a gene selected from the group consisting of ENSBTAG00000024663, ENSBTAG00000016057, ENSBTAG00000010732, ENSBTAG00000015635, ENSBTAG00000015632, ENSBTAG00000014707, ENSBTAG00000014537 and ENSBTAG00000037523, cf. table 20.

### BTA18

In another embodiment of the invention the at least one genetic marker is located on the bovine chromosome BTA18 in a region delineated by BovineHD Genotyping BeadChip SNP#11892 and SNP#13902 and/or in a region between base nos. 41653211 and 48570545, for example, the marker is BovineHD1800013234 or is BovineHD Genotyping BeadChip SNP#44778431, or is linked to any of said markers.

### BTA19

In another embodiment of the invention the at least one genetic marker is located on the bovine chromosome BTA19 in a region delineated by BovineHD Genotyping BeadChip SNP#12750 and SNP#16762 and/or in a region between base nos. 49013784 and 62339802, for example, the marker is BovineHD1900015719 or is BovineHD Genotyping BeadChip SNP#55615219, or is linked to any of said markers.

In one embodiment, the genetic marker is located on the bovine chromosome BTA19 in a region between 55-58 Mb, for example the marker is Chr19_{—}55296191 and/or the trait is mastitis resistance, such as SCS3. In one embodiment, the genetic marker is selected from the group consisting of Chr19_{—}57164311, Chr19_{—}55461224, BovineHD1900015719, Chr19_{—}57418222, BovineHD1900015719, Chr19_{—}55296191, Chr19_{—}55296191, Chr19_{—}55296191 and Chr19_{—}55296191 (cf. table 21), and/or the genetic marker allele associated with increased mastitis resistance, and/or the specific trait is as indicated in table 21.

In one embodiment, the genetic marker is located in a gene selected from the group consisting of ENSBTAG00000013677, ENSBTAG00000005104 and ENSBTAG00000044443; cf. table 22.

### BTA20

In another embodiment of the invention the at least one genetic marker is located on the bovine chromosome BTA20 in a region delineated by BovineHD Genotyping BeadChip SNP#7852 and SNP#14407 and/or in a region between base nos. 28291423 and 55744850, for example, the marker is BovineHD2000010279 or is BovineHD Genotyping BeadChip SNP#35981673, or is linked to any of said markers.

In one embodiment, the genetic marker is located on the bovine chromosome BTA20 in a region between 32-40 Mb, for example the marker is Chr20_{—}35965955 and/or the trait is mastitis resistance, such as CM2. In one embodiment, the genetic marker is selected from the group consisting of Chr20_{—}34269660, Chr20_{—}35965955, Chr20_{—}35965955, Chr20_{—}35914181, Chr20_{—}35965955, Chr20_{—}35969130, Chr20_{—}35865606, Chr20_{—}35914086 and Chr20_{—}35543794 (cf. table 23), and/or the genetic marker allele associated with increased mastitis resistance, and/or the specific trait is as indicated in table 23. In one embodiment, the marker is Chr20_{—}35965955 and the allele associated with mastitis resistance is the A-allele.

In one embodiment, the genetic marker is located in a gene selected from the group consisting of ENSBTAG00000010423, ENSBTAG00000014972, ENSBTAG00000016149, ENSBTAG00000006697, ENSBTAG00000033107, ENSBTAG00000011766 and ENSBTAG00000014177, cf. table 25.

In one specific embodiment, the at least one genetic marker is located in the Caspase recruitment domain-containing protein 6 gene (CARD6) on BTA20. Thus, in one preferred embodiment, the genetic marker associated with at least one trait indicative of mastitis, such as clinical mastitis, for example CM11, is located in the CARD6 gene, in particular in the coding region of NPFFR2. In one embodiment, the genetic marker associated with one or more mastitis traits is the rs133218364 SNP, which is located in the CARD6 gene on BTA20; cf. SEQ ID NO: 2. This SNP is a T-C substitution. However, as alternative SNPs located within the CARD6 gene are strongly coupled to the rs133218364 SNP, any genetic marker polymorphism located in the CARD6 gene is associated with a trait indicative of mastitis. Thus, the present invention relates to methods of determining mastitis and/or a breeding value as well as methods for selected cattle for breeding, and kits, wherein the at least one genetic marker is located in the CARD6 gene or is genetically coupled to the CARD6 gene, and in one preferred embodiment, the at least one genetic marker is the rs133218364 SNP and/or any genetic marker polymorphism genetically coupled thereto. Thus, in one embodiment, the genetic marker is the T/C SNP located in the CARD6 gene, wherein the T allele is associated with mastitis and the C allele is associated with resistance to mastitis.

In another specific embodiment, the at least one genetic marker is located in the Leukemia inhibitory factor receptor gene (LIFR) on BTA20, or the flanking sequences thereof, such as 5000 bp upstream or downstream of the LIFR gene. In one preferred embodiment, the genetic marker associated with at least one trait indicative of mastitis, such as clinical mastitis, for example CM11, is located in the LIFR gene or the flanking sequences, in particular within 5000 bp downstream of the LIFR gene coding region. In one embodiment, the genetic marker associated with one or more mastitis traits is the rs133596506 SNP, which is located 3323 bp downstream of the LIFR gene on BTA20; cf. SEQ ID NO: 3. This SNP is a T-C substitution. However, as alternative SNPs located within the LIFR gene and its flanking regions are strongly coupled to the rs133596506 SNP, any genetic marker polymorphism located in the LIFR gene and its flanking regions is associated with a trait indicative of mastitis. Thus, the present invention relates to methods of determining mastitis and/or a breeding value as well as methods for selected cattle for breeding, and kits, wherein the at least one genetic marker is located in the LIFR gene or its flanking regions is genetically coupled to the LIFR gene, and in one preferred embodiment, the at least one genetic marker is the rs133596506 SNP and/or any genetic marker polymorphism genetically coupled thereto. Thus, in one embodiment, the genetic marker is the T/C SNP located in the LIFR gene or its flanking regions, wherein the C allele is associated with mastitis and the T allele is associated with resistance to mastitis.

### Detection

The method according to the present invention for determining mastitis resistance of a bovine subject comprises detecting in a sample from said bovine subject the presence or absence of at least one genetic marker allele that is associated with at least one trait indicative of mastitis resistance of said bovine subject and/or off-spring therefrom. Specific genetic markers associated with mastitis resistance are provided elsewhere herein. The genetic markers, including microsatellite markers and/or SNPs, or a complementary sequence as well as transcriptional (mRNA) and translational products (polypeptides, proteins) therefrom may be identified by any method known to those of skill within the art.

It will be apparent to the person skilled in the art that there are a large number of analytical procedures which may be used to detect the presence or absence of variant nucleotides at one or more of positions mentioned herein in the specified region. Mutations or polymorphisms within or flanking the specified region can be detected by utilizing a number of techniques. Nucleic acid from any nucleated cell can be used as the starting point for such assay techniques, and may be isolated according to standard nucleic acid preparation procedures that are well known to those of skill in the art. In general, the detection of allelic variation requires a mutation discrimination technique, optionally an amplification reaction and a signal generation system.

A number of mutation detection techniques are listed below. Some of the methods listed are based on the polymerase chain reaction (PCR), wherein the method according to the present invention includes a step for amplification of the nucleotide sequence of interest in the presence of primers based on the nucleotide sequence of the variable nucleotide sequence. The methods may be used in combination with a number of signal generation systems, a selection of which is listed further below.

| | |
|---|---|
| General techniques | DNA sequencing, Sequencing by hybridisation, SNAP- |
| | shot |
| Scanning techniques | Single-strand conformation polymorphism analysis, De- |
| | naturing gradient gel electrophoresis, Temperature gradi- |
| | ent gel electrophoresis, Chemical mismatch cleavage, |
| | cleavage, heteroduplex analysis, enzymatic mismatch |
| | cleavage |
| Hybridisation based | Solid phase hybridisation: Dot blots, Multiple allele |
| techniques | specific diagnostic assay (MASDA), Reverse dot blots, |
| | Oligo-nucleotide arrays (DMA Chips) |
| | Solution phase hybridisation: Taqman -U.S. Pat. No. |
| | 5,210,015 & 5,487,972 (Hoffmann-La Roche), Molecular |
| | Beacons -- Tyagi et al (1996), Nature Biotechnology, 14, |
| | 303; WO 95/13399 (Public Health Inst., New York), Light- |
| | cycler, optionally in combination with Fluorescence reso- |
| | nance energy transfer (FRET). |
| Extension based tech- | Amplification refractory mutation system (ARMS), Ampli- |
| niques | fication refractory mutation system linear extension |
| | (ALEX) - European Patent No. EP 332435 B1 (Zeneca |
| | Limited), Competitive oligonucleotide priming system |
| | (COPS) - Gibbs et al (1989), Nucleic Acids Research, 17, |
| | 2347. |
| Incorporation based | Mini-sequencing, Arrayed primer extension (APEX) |
| techniques | |
| Restriction Enzyme | Restriction fragment length polymorphism (RFLP), Re- |
| based techniques | striction site generating PCR |
| Ligation based tech- | Oligonucleotide ligation assay (OLA) |
| niques | |
| Other | Invader assay |
| Various Signal Genera- | Fluorescence: |
| tion or Detection Sys- | Fluorescence resonance energy transfer (FRET), Fluo- |
| tems | rescence quenching, Fluorescence polarisation-United |
| | Kingdom Patent No. 2228998 (Zeneca Limited) |
| Other | Chemiluminescence, Electrochemiluminescence, Raman, |
| | Radioactivity, Colorimetric, Hybridisation protection as- |
| | say, Mass spectrometry |

Further amplification techniques are found elsewhere herein. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2nd Edition by Newton & Graham, BIOS Scientific Publishers Limited, 1997.

The detection of genetic markers can according to one embodiment of the present invention be achieved by a number of techniques known to the skilled person, including typing of microsatellites or short tandem repeats (STR), restriction fragment length polymorphisms (RFLP), detection of deletions or insertions, random amplified polymorphic DNA (RAPIDs) or the typing of single nucleotide polymorphisms by methods such as restriction fragment length polymerase chain reaction, allele-specific oligomer hybridisation, oligomer-specific ligation assays, hybridisation with PNA or locked nucleic acids (LNA) probes.

In one embodiment, the methods of the invention comprise amplifying a genetic region comprised in the sample provided from the bovine subject. Thus, specific methods may include amplifying a genetic region comprising a genetic marker of the invention, and detecting that amplification product.

In another preferred embodiment, the genetic marker is detected by DNA array methods. It is, for example, possible to genotype large numbers of SNP markers simultaneously using commercially available SNP genotyping kits. Such kits are for example the bovineSNP50 beadchip SNP kit provided by Illumina Inc., and the BovineHD BeadChip from Illumina Inc. Both of these kits are preferred for SNP genotyping according to the present invention.

A primer of the present invention is a nucleic acid molecule sufficiently complementary to the sequence on which it is based and of sufficiently length to selectively hybridise to the corresponding region of a nucleic acid molecule intended to be amplified. The primer is able to prime the synthesis of the corresponding region of the intended nucleic acid molecule in the methods described above. Similarly, a probe of the present invention is a molecule for example a nucleic acid molecule of sufficient length and sufficiently complementary to the nucleic acid sequence of interest which selectively binds to the nucleic acid sequence of interest under high or low stringency conditions. The genetic marker associated with mastitis resistance according to the present invention can be detected by a number of methods known to those of skill within the art. For example, the genetic marker may be identified by genotyping using a method selected from the group consisting of single nucleotide polymorphisms (SNPs), microsatellite markers, restriction fragment length polymorphisms (RFLPs), DNA chips, amplified fragment length polymorphisms (AFLPs), randomly amplified polymorphic sequences (RAPDs), sequence characterised amplified regions (SCARs), cleaved amplified polymorphic sequences (CAPSs), nucleic acid sequencing, and microsatellite genotyping.

In a preferred embodiment, the genetic markers associated with mastitis resistance traits as disclosed in the present invention is detected by SNP or microsatellite genotyping. SNP or microsatellite genotyping may be performed by amplification of the SNP or microsatellite marker by sequence specific oligonucleotide primers, and subsequent analysis of the amplification product, in terms of for example length, quantity and/or sequence of the amplification product.

Specifically, the at least one genetic marker according to the present invention may be detected by use of at least one oligonucleotide comprising between 5 and 100 consecutive nucleotides, such as between 10 and 30 consecutive nucleotides, or at least 5, such as 6, 7, 8, 9, 10, 11, 12, 13, 14, 15, 16, 17, 18, 19, 20, 21, 22, 23, 24 or at least 25 consecutive nucleotides of the NPFFR2 gene, such as SEQ ID NO: 1, or a nucleic acid sequence at least 70% identical thereto, such as at least 75, 76, 77, 78, 79, 80, 81, 82, 83, 84, 85, 86, 87, 88, 89, 90, 91, 92, 93, 94, 95, 96, 97, 98, such as at least 99% thereto.

In one embodiment of the methods and kits of the present invention, the genetic marker is detected by using an oligonucleotide primer or probe capable of recognizing at least one SNP selected from the group of SNPs set forth in column 10 of table 2. The oligonucleotide may be used as a primer in a nucleic acid amplification reaction and/or the oligonucleotide may be used as a probe in a hybridization detection technique.

The primers of the present invention may be used individually or in combination with one or more primers or primer pairs, such as any primer of the present invention.

The design of such primers or probes will be apparent to the molecular biologist of ordinary skill. Such primers are of any convenient length such as up to 50 bases, up to 40 bases, more conveniently up to 30 bases in length, such as for example 8-25 or 8−15 bases in length. In general such primers will comprise base sequences entirely complementary to the corresponding wild type or variant locus in the region. However, if required one or more mismatches may be introduced, provided that the discriminatory power of the oligonucleotide probe is not unduly affected. The primers/probes of the invention may carry one or more labels to facilitate detection.

In one embodiment, the primers and/or probes are capable of hybridizing to and/or amplifying a subsequence hybridizing to a single nucleotide polymorphism containing the sequence delineated by the markers as shown herein.

The primer nucleotide sequences of the invention further include: (a) any nucleotide sequence that hybridizes to a nucleic acid molecule comprising a genetic marker sequence or its complementary sequence or RNA products under stringent conditions, e.g., hybridization to filter-bound DNA in 6× sodium chloride/sodium citrate (SSC) at about 45° C. followed by one or more washes in 0.2×SSC/0.1% Sodium Dodecyl Sulfate (SDS) at about 50-65° C., or (b) under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel F. M. et al., eds., 1989, Current Protocols in Molecular Biology, Vol. I, Green Publishing Associates, Inc., and John Wiley & sons, Inc., New York, at pp. 6.3.1-6.3.6 and 2.10.3). Preferably the nucleic acid molecule that hybridizes to the nucleotide sequence of (a) and (b), above, is one that comprises the complement of a nucleic acid molecule of the genomic DNA comprising the genetic marker sequence or a complementary sequence or RNA product thereof.

Among the nucleic acid molecules of the invention are deoxyoligonucleotides (“oligos”) which hybridize under highly stringent or stringent conditions to the nucleic acid molecules described above. In general, for probes between 14 and 70 nucleotides in length the melting temperature (TM) is calculated using the formula:

Tm(° C.)=81.5+16.6(log [monovalent cations(molar)])+0.41(% G+C)−(500/N)

where N is the length of the probe. If the hybridization is carried out in a solution containing formamide, the melting temperature is calculated using the equation Tm(° C.)=81.5+16.6(log [monovalent cations (molar)])+0.41(% G+C)−(0.61% formamide)−(500/N) where N is the length of the probe. In general, hybridization is carried out at about 20-25 degrees below Tm (for DNA-DNA hybrids) or 10-15 degrees below Tm (for RNA-DNA hybrids).

Exemplary highly stringent conditions may refer, e.g., to washing in 6×SSC/0.05% sodium pyrophosphate at 37° C. (for about 14-base oligos), 48° C. (for about 17-base oligos), 55° C. (for about 20-base oligos), and 60° C. (for about 23-base oligos).

Accordingly, the invention further provides nucleotide primers or probes which detect the polymorphisms of the invention. The assessment may be conducted by means of at least one nucleic acid primer or probe, such as a primer or probe of DNA, RNA or a nucleic acid analogue such as peptide nucleic acid (PNA) or locked nucleic acid (LNA).

According to one aspect of the present invention there is provided an allele-specific oligonucleotide probe capable of detecting a polymorphism at one or more of positions in the delineated regions.

The allele-specific oligonucleotide probe is preferably 5-50 nucleotides, more preferably about 5-35 nucleotides, more preferably about 5-30 nucleotides, more preferably at least 9 nucleotides.

Determination of Association with Mastitis

In order to detect if a genetic marker is present in the genetic material, standard methods well known to persons skilled in the art may be applied, e.g. by the use of nucleic acid amplification. In order to determine if the genetic marker is genetically linked to mastitis resistance traits, a permutation test can be applied (Doerge and Churchill, 1996), or the Piepho-method can be applied (Piepho, 2001). The principle of the permutation test is well described by Doerge and Churchill (1996), whereas the Piepho-method is well described by Piepho (2001). Significant linkage in the within family analysis using the regression method, a 10000 permutations were made using the permutation test (Doerge and Churchill, 1996). A threshold at the 5% chromosome wide level was considered to be significant evidence for linkage between the genetic marker and the mastitis resistance and somatic cell count traits. In addition, the QTL was confirmed in different sire families. For the across family analysis and multi-trait analysis with the variance component method, the Piepho-method was used to determine the significance level (Piepho, 2001). A threshold at the 5% chromosome wide level was considered to be significant evidence for linkage between the genetic marker and the mastitis resistance and somatic cell count traits.

### Method for Selecting a Bovine Subject

In one aspect, the present invention further relates to a method for selecting a bovine subject for breeding purposes. This method for selecting a bovine subject for breeding purposes comprises determining resistance to mastitis of said bovine subject and/or off-spring therefrom by any method as defined herein, such as determining resistance to mastitis in a bovine subject, by detecting in a sample from said bovine subject the presence or absence of at least one genetic marker as defined herein.

The purpose of the method is to select those bovine subjects with the best breeding value for breeding. For example, selection of bovine subjects for breeding according to the present invention serve to increase the mean breeding value of the next generation of bovine subjects, compared to the mean breeding value of the previous (parent) generation of bovine subjects.

In one embodiment, the method of the present invention for selecting a bovine subject for breeding purposes comprises estimating a breeding value of said selected bovine subject. For example, the breeding value is estimated on the basis of the presence or absence of a genetic marker of the present invention.

### Kit

In one aspect, the present invention relates to a kit, such as a diagnostic kit, for detecting the presence or absence in a bovine subject of at least one genetic marker as described herein, such as a marker associated with resistance to mastitis. In one embodiment, the present invention relates to a diagnostic kit for detecting the presence or absence in a bovine subject of two or more genetic marker alleles as described elsewhere herein, said kit comprising at least one detection member. Specifically, the kit is suitable for detection of the presence or absence of at least one genetic marker allele, such as two or more genetic markers, which are associated with at least one trait indicative of mastitis resistance of said bovine subject and/or off-spring therefrom. Examples of specific traits which are indicative of mastitis resistance are disclosed elsewhere herein. Such traits include, SCS, SCC, and treated cases of clinical mastitis, for example CM11, CM12, CM2, CM3, CM, SCC3, SCC2, SCC1 and/or SCC.

The kit of the invention preferably comprise at least one detection member for determining a genetic marker located in a genomic region as defined herein above.

Detection members of the present invention include any entity, which is suitable for detecting a genetic marker on the genomic (including epigenomic), transcriptional or translational level. Detection members comprise oligonucleotide primers and/or probes, antibodies, aptamers, chemical substances etc.

In one embodiment, the diagnostic kit comprises at least one oligonucleotide for detecting said genetic marker allele in said bovine subject.

In one embodiment, the detection member is an oligonucleotide primer and/or an oligonucleotide probe. In a preferred embodiment, the detection member is an oligonucleotide primer as described elsewhere herein, or an oligonucleotide probe with a sequence corresponding to any oligonucleotide primer as defined herein. The at least one oligonucleotide of the kit preferably comprises or consists of between 5 and 100 consecutive nucleotides, such as between 10 and 30 consecutive nucleotides, or at least 5, such as 6, 7, 8, 9, 10, 11, 12, 13, 14, 15, 16, 17, 18, 19, 20, 21, 22, 23, 24 or at least 25 consecutive nucleotides. In a preferred embodiment, the detection member is an oligonucleotide comprising at least 5 consecutive nucleotides specific for any one of the SNP markers set forth in columns 9 and 10 in the table identified in table 2.

In one aspect, the present invention relates to a kit for use in detecting the presence or absence in a bovine subject of at least one genetic marker associated with resistance to mastitis, comprising at least one detection member for determining a genetic marker located in a region of the bovine genome selected from the group consisting of regions 1-61 of table 2, wherein said regions are delineated by the SNP markers identified in columns 3 and 5, and/or delineated by the genomic position identified in columns 4 and 6.

The genetic markers to be detected by the detection members of the kit of the present invention are disclosed elsewhere herein. Thus, the genetic marker is for example any genetic marker as described herein, such as two or more genetic marker alleles located in a gene selected from the group consisting of the markers mentioned in columns 9 and 10 of table 2. In a preferred embodiment, the genetic marker is located in the NPFFR2 gene, as defined elsewhere herein. Thus, in one embodiment, the kit of the invention comprise at least one detected member capable of detecting a mutation in the NPFFR2 gene, in particular for detecting the chr6_{—}89059253 SNP located at 89,059,253 Bp position on BTA6. The detection member, thus in a preferred embodiment is a nucleic acid sequence comprising between 5 and 100 consecutive nucleotides, such as between 10 and 30 consecutive nucleotides, or at least 5, such as 6, 7, 8, 9, 10, 11, 12, 13, 14, 15, 16, 17, 18, 19, 20, 21, 22, 23, 24 or at least 25 consecutive nucleotides of the NPFFR2 gene, such as SEQ ID NO: 1, or a nucleic acid sequence at least 70% identical thereto, such as at least 75, 76, 77, 78, 79, 80, 81, 82, 83, 84, 85, 86, 87, 88, 89, 90, 91, 92, 93, 94, 95, 96, 97, 98, such as at least 99% thereto. In a preferred embodiment, the nucleic acid sequence comprises the chr6_{—}89059253 SNP, and/or any genetic marker polymorphism coupled thereto.

The kits of the present invention may further comprise at least one reference sample. In one embodiment, said reference sample comprises a nucleic acid sequence comprising a genetic marker associated with mastitis resistance, such as described herein, and in another embodiment, the reference sample comprises a nucleic acid sequence comprising a genetic marker associated with susceptibility to mastitis

The kits of the present invention further comprise in specific embodiments instructions for performance of the detection method of the kit and for the interpretation of the results.

Genotyping of a bovine subject in order to establish the genetic determinants of resistance to mastitis for that subject according to the present invention can be based on the analysis of DNA and/or RNA. One example is genomic DNA which can be provided using standard DNA extraction methods as described herein. The genomic DNA may be isolated and amplified using standard techniques such as the polymerase chain reaction using oligonucleotide primers corresponding (complementary) to the polymorphic marker regions. Additional steps of purifying the DNA prior to amplification reaction may be included. Thus, a diagnostic kit for establishing mastitis resistance and somatic cell count characteristics comprises, in a separate packing, at least one oligonucleotide sequence.

The invention also relates to the use of a kit of the invention for detecting the presence or absence in a bovine subject of at least one genetic marker associated with resistance to mastitis, in particular for detecting any one or more of the markers identified herein. Furthermore, the present invention relates to the use of a kit of the present invention for estimating breeding value in respect of susceptibility to mastitis in a bovine subject.

### Method of Estimating Breeding Value

The present invention also relates to determination of estimated breeding values.

In a large randomly mated population, each individual should on average give birth to two offspring in order to maintain the size of the population. The distribution of the number of offspring in the population has a left skewed binominal distribution (Poisson distributed) with an average value of 2 and variance of 2. Which means that the number of offspring per individual can vary from 0 and upwards, the values 0, 1, 2, 3, 4 and 5 being the most frequent. An estimated breeding value is often called an index (I). The index can be estimated on the basis of information of phenotype values from all possible relatives. A simple regression line or multiple regression can be used. The higher the number of relatives is the better the estimation will be. Correlation between the true breeding value (A) and the index is given the name Accuracy and it has the symbol rAI. The estimated breeding value is based on a theory of linear regression and correlation.

In one aspect, the present invention relates to a method for estimating a breeding value in respect of susceptibility to mastitis in a bovine subject, comprising detecting in a sample from said bovine subject the presence or absence of at least one genetic marker that is associated with at least one trait indicative of mastitis resistance of said bovine subject and/or off-spring therefrom, wherein said at least one genetic marker is located in a region of the bovine genome selected from the group consisting of regions 1-61 of table 2, wherein said regions are delineated by the SNP markers identified in columns 3 and 5, and/or delineated by the genomic position identified in columns 4 and 6. The method preferably comprises detection of one or more of the specific markers associated with mastitis, which are identified elsewhere herein.

The breeding value is in one example determined using a multi-trait random regression model (mtRRM) combined longitudinal TDSCS and binary CM traits, for example having the general description of the model in matrix form:

y=Xb+Hₕh+Kₖk+Zₐa+Zₚp+e,

where: y is a vector with observations on the nine different traits explained above. Vectors b, h, k contain the environmental effects whilst vectors a, and p contain additive genetic and nongenetic animal regression coefficients, respectively.

Environmental effects in the model could be calving age, herd environment and stage of lactation. Both additive genetic and non-genetic animal effects can be modelled by a second order Legendre polynomial for TDSCS and intercept for the other traits leading to a 15×15 (co)variance matrix for each random effect to be estimated. Vector e contains the residuals of the 9 traits.

In order to facilitate accurate estimation, residual (co)variances between CM traits and TDSCS may be assumed to be zero and the residual variance of CM and udder type traits may be set to operationally low values so that part of this variance entered the permanent environmental component. This can facilitate estimation of permanent environmental correlation between CM and the longitudinal trait. The covariance components were estimated using DMU package.

In one embodiment, the breeding value is calculated using a marker-assisted single trait Best Linear Unbiased Prediction (MA-BLUP).

The specific mastitis resistances traits, genetic markers and marker alleles, samples, bovine subjects, detection methods etc. are defined elsewhere herein.

### Selective Breeding

In one aspect, the present invention provides a method for selective breeding of bovine subjects. The method of the invention allows the identification of bovine subjects suitable for selective breeding.

In one embodiment these methods comprise the steps of

a. providing a bovine subject,
b. obtaining a biological sample from said subject,
c. determining the presence in that sample of at least one genetic marker located in a region of the bovine genome selected from the group consisting of regions 1-61 of table 2, wherein said regions are delineated by the SNP markers identified in columns 3 and 5, and/or delineated by the genomic position identified in columns 4 and 6,
d. selecting a bovine subject having in its genome said at least one genetic marker, and
e. using said bovine subject for breeding.

The biological sample could be any suitable sample comprising genetic material, and which is preferably easily obtainable. Sample types are described further elsewhere herein.

The bovine is preferably a male subject, i.e. a bull. For example, when the bovine subject is a bull, the use of the bovine subject for breeding would normally include collecting semen from said bull and using said semen for artificial insemination of one or more heifers or cows.

However, the presence of the relevant genetic marker(s) may also be determined in cows and heifers according to the method of the invention.

### EXAMPLES

### Example 1

### Fine-Mapping of Clinical Mastitis and Somatic Cell Score QTL in Dairy Cattle

### Introduction

Genome-wide linkage analysis was until recently the method of choice for quantitative trait loci (QTL) genome scan in cattle due to availability of large half-sib family structure. Linkage analysis is the method traditionally used to identify genes for phenotypes exhibiting Mendelian inheritance. For complex phenotypes such as quantitative traits, linkage analysis has only had limited success. In linkage analysis there are a few opportunities for recombination to occur within families and pedigree with known ancestry, resulting in relatively low mapping resolution which limits the candidate polymorphism search. In the contrary, association mapping (linkage disequilibrium mapping) has emerged as a powerful tool to resolve complex trait variation down to the sequence level by exploiting historical recombination events at the population level for high resolution mapping. In this approach markers/haplotypes with predicting ability in the general population for a trait of interest are identified. Such markers and haplotypes could be used directly for marker-based selection. Typically genome scans are used to map QTL for which some test statistic exceeds a pre-defined threshold value. Although the threshold level can be chosen to be very conservative, a probability that the QTL in reality represents a type I error remains. Therefore, results from QTL studies should be confirmed in an independent analysis before being used in subsequent fine mapping experiments or in marker-assisted selection. If the results from linkage analysis can be confirmed by an association study, it will also provide credibility to the detected QTL.

Lund et al (2008) mapped QTL for clinical mastitis and somatic cell score in Danish Holstein cattle using linkage analysis. These authors used data on 356 microsatellite markers spread across all autosomes with an average marker spacing of 8.6 cM. Nonetheless, the QTL regions reported were quite long (more than 20 cM for some QTL). Such large QTL regions along with family-specific marker-QTL associations limit the usability of their result for practical animal breeding as well as for candidate polymorphism searches. Thus, a need to map QTL to narrower genomic regions remains because inclusion of QTL information in selection decisions requires fine-mapping of causal polymorphisms. In this example, association mapping was carried out for 6 mastitis traits in cattle using dense SNP markers.

### Materials and Methods

### Genotyping

A total of 2,531 Danish and Swedish Holstein bulls were genotyped using the bovineSNP50 beadchip (Illumina®). Only SNPs with minor allele frequency equal to or higher than 0.05 and average GC score of at least 0.65 were retained for the analysis. Thus total of 36,387 SNPs on 29 bovine autosomes (BTAs) were selected for association analyses. Individual SNP types with GC score less than 0.6 were dropped. The number of SNPs included for analysis varied from 675 on BTA28 to 2,320 on BTA1. The details on the genotyping platform and quality control for SNPs are described by Sahana et al. (2010a). The SNP positions within a chromosome were based on the Bos taurus genome assembly (Btau_{—}4.0, Liu et al. 2009).

### Phenotypic Data

Single trait breeding values (STBV) were used as phenotypes in this analysis. Six mastitis related STBVs were analyzed for association with SNPs. Single-trait breeding values were calculated for each animal using best linear unbiased prediction (BLUP) procedures and a sire model by the Nordic Cattle Genetic Evaluation. For definitions and models used in breeding value prediction, see http://www.nordicebv.info, except that the correlation to other traits was set to 0 to avoid information from phenotypes of correlated traits to affect results of any particular trait. Also, only sire-son and sonoffspring relationships were included, effectively producing a sire model. The STBV were adjusted for the same systematic environmental effects as in the official routine evaluations. Clinical mastitis was defined as a binary trait, mastitis treatment (1) or not (0) within four time periods: the incidence of mastitis from −15 to 50 days in first lactation (CM11), 51 to 305 days in first lactation (CM12), −15 to 305 days in second lactation (CM2), −15 to 305 days in third lactation (CM3), all measure as binary trait. The STBVs for the four mastitis traits are weighted together by the following relative weights: CM=0.25*CM11+0.25*CM12+0.3*CM2+0.2*CM3 to form a mastitis resistance index (CM) (Johansson et al. 2007), standardized to a mean of 100 and a standard deviation of 10. Somatic cell score (SCS) is an important trait for the estimation of breeding values for udder health. SCS is an index of average log somatic cell count from 5 to 170 days from first three lactations with relative weights of 0.5, 0.3 and 0.2 for first, second and third lactation respectively (Johansson et al. 2007). The number of STBVs available for analysis among the genotyped animals were 1671 for CM11, CM12 1668, CM2 1669, CM3 1544, CM 2098 and SCS 1671.

### Statistical Methods for Association Analysis

Mixed model: The mixed model analysis as proposed by Yu et al. (2006) was used for association analyses. In this approach, a polygenic genetic effect was fitted as a random effect. Single SNPs were successively included as fixed effect in the model. The model was:

y=μ1+αs+Zu+e

where y is a vector of observed phenotypes (STBV), μ is a shared fixed effect, 1 is a vector of ones, α is allele substitution effect of the SNP, s is an incidence vector with elements 0, 1 or 2 relating α to the individuals, Z is a matrix relating records to individuals, u is a vector of additive polygenic effects and e is a vector of random residual effects. The random variables u, and e are assumed to be multivariate normally distributed. u has mean 0 and covariance matrix σ_{g}²A, where σ_{g}² is the polygenic genetic variance and A is the additive relationship matrix derived from pedigree. e has mean 0 and covariance matrix □ₑ²I, where □ₑ² is the residual variance and I is the identity matrix. The analysis was carried out using the software package DMU (http://gbiagrsci.dk/dmu/). Significance of each marker's effect was tested using a t-test against a null hypothesis of α=0.

Significance Test:

For control of the family-wise error rate (FWER), the Bonferroni correction was applied. The Bonferroni correction controls FWER (α)=1−(1−aᵢ)^{m} αᵢm, where αᵢ is the individual test rejection level and m is the number of tests. The 5% chromosome-wise significance thresholds ranged from the point wise p-value of 2.16×10⁻⁵ on BTA1 to 7.41×10⁻⁵ on BTA28, or 4.67 to 4.13 in the −log₁₀ transformed scale. Bonferroni correction is very conservative (Han et al. 2009) as it does not take account of correlation (linkage disequilibrium) among SNPs. We used a liberal significance threshold of 10⁻⁴ for the QTL regions where QTL have previously been identified by Lund et al. (2008) who used linkage analyses with microsatellite markers for QTL mapping. In the following sections a significant association will mean chromosome-wise significant, and a suggestive association means a point wise p-value less than 10⁻⁴.

Marking the QTL Region:

Normally multiple SNPs in the vicinity of a QTL are expected to yield significant results in a single SNP analysis. This is because all SNPs that are physically located near the causal factor will tend to be in linkage disequilibrium. This effect declines with genetic distance and also depends on minor allele frequencies. In this study, QTL regions were demarcated subjectively. Starting at the most significant SNP, the QTL region was extended left and right until a region was reached where all markers had −log(p) values below 3. I.e. that the QTL thus demarcated may contain one or more non-significant markers. To compare results from the present study with the earlier ones, we took the maker positions from Btau_{—}4.0. If the maker location was not available in Btau_{—}4.0, we have reported marker and given the position in cM from MARC table [http://www.marc.usda.gov/genome/cattle/cattle.html].

### Results

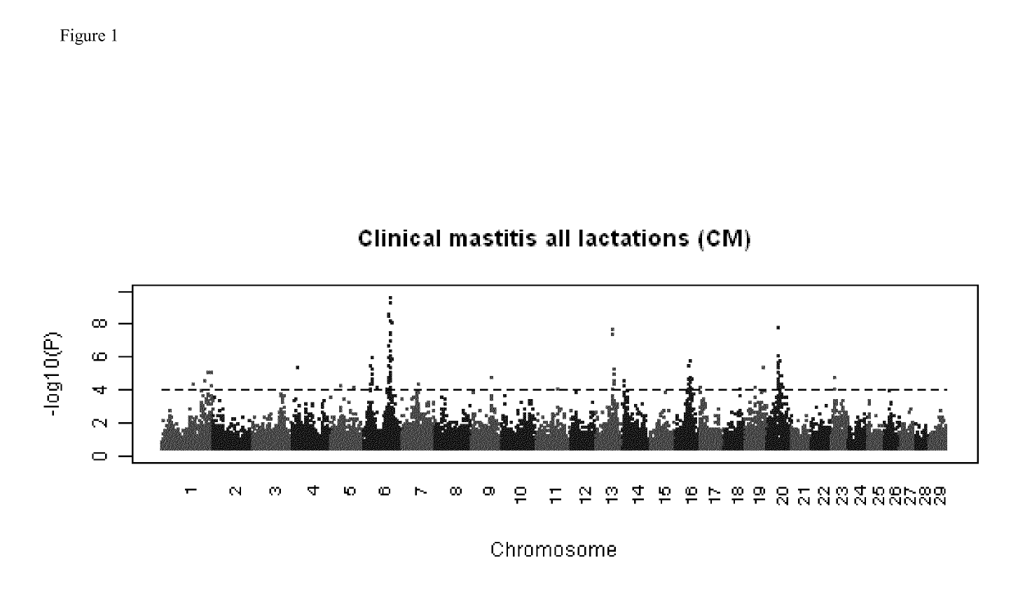

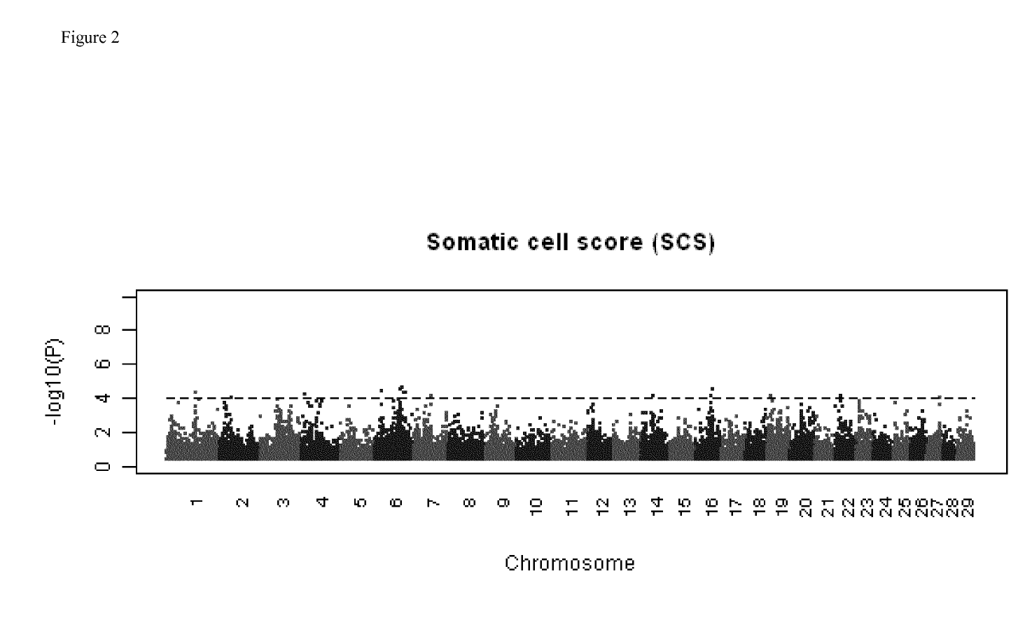

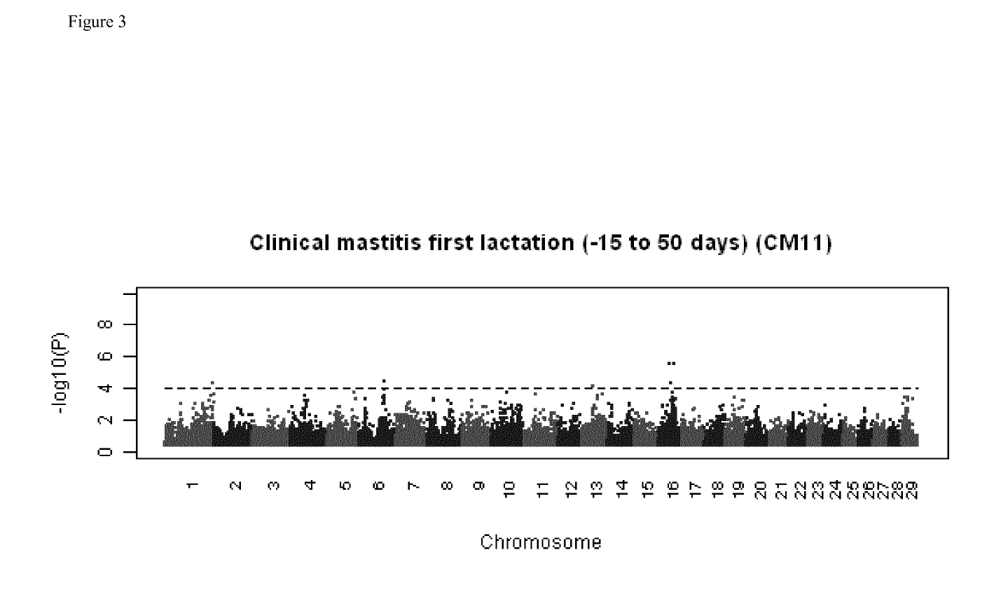

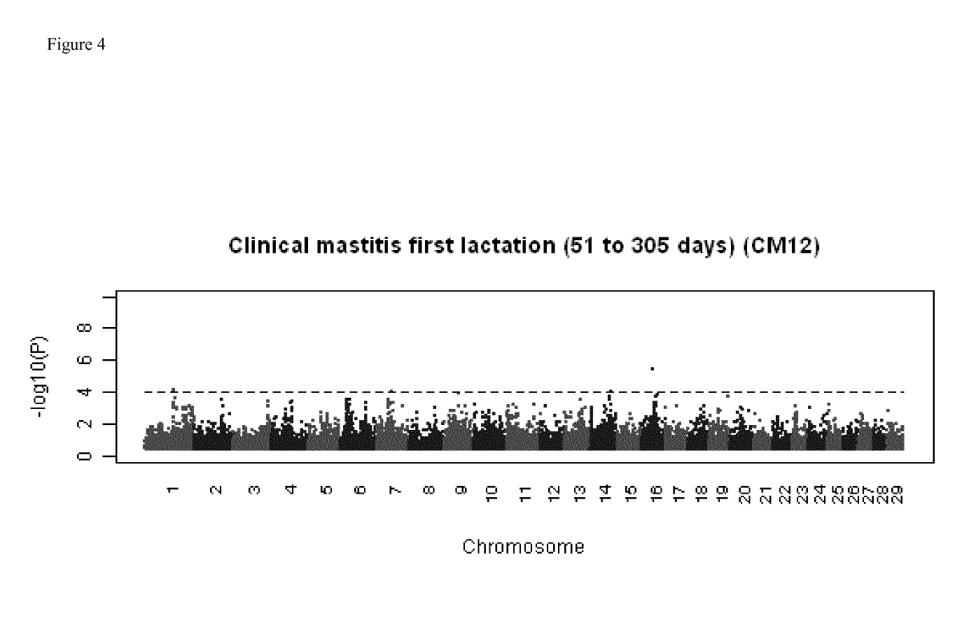

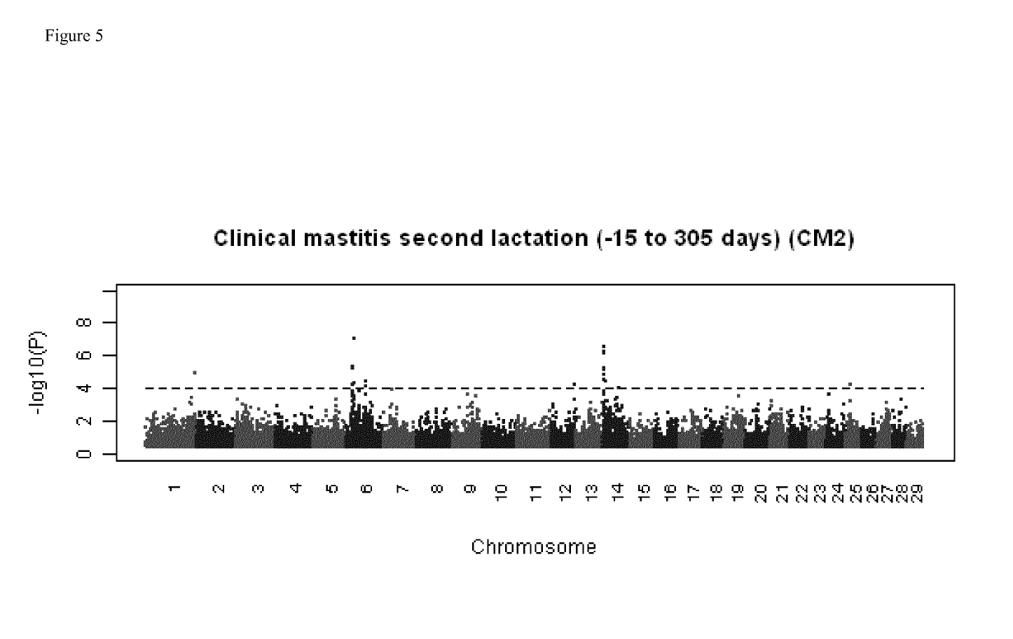

The present genome-wide association study (GWAS) identified 9 chromosome-wise significant QTL for clinical mastitis and somatic cell score on 8 chromosomes in Danish and Swedish Holstein cattle (Table 3). We have presented 92 SNP×trait combinations which showed chromosome-wise significant association and out of then 24 combinations crossed genome-wide significance threshold (Supplementary Table 3). Most of the genome-wide significant associations were observed for CM and four SNP showed genome-wide significant association with CM2. Five SNPs showed significant association with more than one mastitis trait. The signal plots (FIGS. 1 to 6) give an overview how the SNPs association are located across the genome and also help to visualize if the QTL on the genome location affecting more than one trait. The most highly significant signal was observed on BTA6. Here a highly significant association with several mastitis traits was observed. The strongest signal was for CM followed by CM2, SCS and CM11. Consistent results across traits for association were observed on BTA16 for SCS, CM11, CM12, and CM, and on BTA1 for SCS, CM11, CM12, CM2 and CM. Confirmation of QTL at the same chromosomal locations across several mastitis traits was also observed on BTA14.

TABLE 3
Quantitative trait loci (QTL) detected by association analysis for
mastitis traits with the most significant SNPs and the QTL region.

| | QTL region | Most significant SNP | | | Traits with significant/ |
|---|---|---|---|---|---|
| Chr. | (Mb) | Name | Pos (Bp) | −log10(P) | suggestive association |
| 1 | 148.3-160.9 | ss86284888 | 159167781 | 5.077 | CM, CM11, CM2 |
| 4 | 14.0-25.7 | rs41649041 | 19718653 | 5.292 | CM, SCS |
| 6 | 20.5-27.8 | ss61565956 | 25195079 | 7.083 | CM, CM2, SCS |
| 6 | 85.0-90.7 | ss86341106 | 89212073 | 9.535 | CM, CM11, SCS |
| 13 | 57.5-61.9 | ss86317725 | 57728100 | 7.653 | CM, CM11, CM12 |
| 14 | 0.1-2.8 | ss86328358 | 679601 | 6.488 | CM, CM2 |
| 16 | 46.3-55.1 | rs41812941 | 50838131 | 5.735 | CM, CM11, SCS |
| 19 | 51.2-61.3 | ss86327354 | 54763344 | 5.314 | CM, CM12, CM2 |
| 20 | 34.1-44.3 | rs41940571 | 37740343 | 7.786 | CM, SCS |

TABLE 4
SNP showing chromosome-wise significant association with mastitis traits. The genome-wide
significant SNPs (which are preferred markers of the present invention) are in bold font.

| | | Position | | | | |
|---|---|---|---|---|---|---|
| chr | SNP | (Bp) | Trait | alpha | se | -log₁₀(P) |
| 1 | ss86328743 | 150055732 | CM | −0.783 | 0.170 | 5.01 |
| 1 | rs41618669 | 157571776 | CM2 | 0.244 | 0.053 | 4.95 |
| 1 | ss86284888 | 159167781 | CM | −0.893 | 0.192 | 5.08 |
| 1 | rs41580905 | 160353510 | CM | −2.522 | 0.543 | 5.07 |
| 4 | rs41649041 | 19718653 | CM | −1.543 | 0.324 | 5.29 |
| 6 | rs43706944 | 20565525 | CM2 | −0.247 | 0.052 | 5.30 |
| 6 | rs42189699 | 20586033 | CM2 | 0.247 | 0.052 | 5.28 |
| 6 | rs42553026 | 22210179 | CM | −0.734 | 0.153 | 5.38 |
| 6 | rs41664497 | 25195079 | CM | −1.418 | 0.310 | 4.94 |
| 6 | rs41664497 | 25195079 | CM2 | 0.581 | 0.104 | 7.08 |
| 6 | ss86290235 | 26706544 | CM | 0.885 | 0.176 | 5.90 |
| 6 | ss86340493 | 27761080 | CM | −0.698 | 0.159 | 4.57 |
| 6 | ss86305923 | 27786722 | CM | 2.455 | 0.554 | 4.65 |
| 6 | ss86330005 | 29212237 | CM | −1.146 | 0.244 | 5.19 |
| 6 | ss86340725 | 81134003 | CM | 0.760 | 0.172 | 4.62 |
| 6 | rs29015635 | 81958670 | CM | 0.810 | 0.161 | 5.89 |
| 6 | rs42895750 | 82226494 | CM | 1.350 | 0.303 | 4.71 |
| 6 | ss117968104 | 84194536 | CM | 1.513 | 0.315 | 5.40 |
| 6 | rs29017739 | 85040979 | CM | −2.016 | 0.331 | 8.42 |
| 6 | rs29001782 | 86128028 | CM | 0.949 | 0.154 | 8.55 |
| 6 | rs41588957 | 86467725 | CM | −0.837 | 0.156 | 6.62 |
| 6 | ss86307579 | 87255541 | CM | −0.883 | 0.160 | 6.98 |
| 6 | ss86317213 | 87879378 | CM | −0.788 | 0.156 | 5.90 |
| 6 | rs41610991 | 87904281 | CM | −0.880 | 0.154 | 7.44 |
| 6 | ss117968170 | 88263655 | CM | 0.764 | 0.173 | 4.60 |
| 6 | ss117968764 | 88326005 | CM | 0.720 | 0.151 | 5.29 |
| 6 | ss117968030 | 88370145 | CM | −1.418 | 0.319 | 4.67 |
| 6 | ss117968525 | 88427760 | CM | −0.724 | 0.159 | 4.87 |
| 6 | rs29019575 | 88946762 | CM | −0.891 | 0.192 | 5.07 |
| 6 | ss117968738 | 88983536 | CM | 0.974 | 0.172 | 7.36 |
| 6 | ss86326721 | 89030230 | CM | 0.691 | 0.157 | 4.57 |
| 6 | ss86341106 | 89212073 | cell | 0.010 | 0.002 | 4.60 |
| 6 | ss86341106 | 89212073 | CM | −1.071 | 0.164 | 9.53 |
| 6 | rs29010419 | 89274693 | CM | −0.959 | 0.217 | 4.64 |
| 6 | rs29022799 | 89603521 | CM | 1.183 | 0.248 | 5.31 |
| 6 | ss86278591 | 89668441 | CM | −2.444 | 0.468 | 6.29 |
| 6 | ss86337596 | 89774923 | CM | −0.972 | 0.152 | 9.22 |
| 6 | rs43338539 | 89838828 | CM | 0.917 | 0.179 | 6.05 |
| 6 | ss86296213 | 90008100 | CM | 1.144 | 0.190 | 8.18 |
| 6 | rs42766480 | 90075264 | CM | 0.916 | 0.167 | 6.89 |
| 6 | rs41617692 | 90670191 | CM | −1.155 | 0.232 | 5.76 |
| 6 | ss117963883 | 94872475 | CM | −0.987 | 0.166 | 8.02 |
| 6 | rs43475842 | 97726008 | CM | −1.680 | 0.336 | 5.81 |
| 7 | rs29019286 | 55023686 | CM | −0.697 | 0.164 | 4.33 |
| 9 | ss86292503 | 75920644 | CM | 0.757 | 0.169 | 4.75 |
| 13 | ss86317725 | 57728100 | CM | −0.858 | 0.148 | 7.65 |
| 13 | ss86290731 | 57750019 | CM | 0.846 | 0.149 | 7.32 |
| 13 | ss86332750 | 60565842 | CM | 0.683 | 0.149 | 4.94 |
| 13 | ss86335834 | 61476511 | CM | 0.682 | 0.156 | 4.56 |
| 13 | ss86340346 | 61851139 | CM | −0.657 | 0.152 | 4.45 |
| 13 | ss105239139 | 61885421 | CM | −0.721 | 0.153 | 5.20 |
| 14 | ss117971362 | 76704 | CM2 | 0.246 | 0.052 | 5.23 |
| 14 | ss86287919 | 236533 | CM2 | −0.282 | 0.054 | 6.25 |
| 14 | ss86329615 | 443936 | CM2 | −0.278 | 0.054 | 6.09 |
| 14 | ss86301882 | 596340 | CM2 | 0.297 | 0.068 | 4.54 |
| 14 | ss86328358 | 679601 | CM2 | −0.279 | 0.052 | 6.49 |
| 14 | ss117971370 | 1461084 | CM2 | 0.251 | 0.054 | 5.15 |
| 14 | ss117971325 | 1490177 | CM2 | 0.237 | 0.053 | 4.79 |
| 14 | ss86339873 | 1913107 | CM2 | 0.267 | 0.061 | 4.57 |
| 14 | ss117971671 | 2757890 | CM | −0.681 | 0.156 | 4.54 |
| 14 | ss117971176 | 4477035 | CM2 | −0.246 | 0.057 | 4.41 |
| 16 | rs41807595 | 41214862 | CM12 | 0.269 | 0.056 | 5.39 |
| 16 | rs41807595 | 41214862 | CM11 | 0.191 | 0.039 | 5.49 |
| 16 | rs29023167 | 44203083 | CM | 0.706 | 0.159 | 4.64 |
| 16 | ss86303613 | 46324306 | CM | −0.883 | 0.183 | 5.45 |
| 16 | ss86283374 | 47856310 | CM | 0.882 | 0.206 | 4.38 |
| 16 | ss86328473 | 47965588 | CM | −0.895 | 0.204 | 4.58 |
| 16 | ss86307986 | 48992727 | CM | 1.580 | 0.351 | 4.77 |
| 16 | rs41603818 | 49348430 | CM | −1.788 | 0.399 | 4.74 |
| 16 | rs41812941 | 50838131 | CM | 0.786 | 0.158 | 5.73 |
| 16 | ss105262977 | 54985553 | cell | −0.012 | 0.003 | 4.54 |
| 16 | ss105262977 | 54985553 | CM | 0.916 | 0.207 | 4.66 |
| 16 | rs42465037 | 55087523 | CM11 | −0.386 | 0.080 | 5.48 |
| 19 | ss86327354 | 54763344 | CM | 0.747 | 0.157 | 5.31 |
| 20 | ss86327432 | 34080608 | CM | −0.852 | 0.197 | 4.46 |
| 20 | ss61484557 | 34367588 | CM | −0.878 | 0.178 | 5.66 |
| 20 | rs42329877 | 35113127 | CM | 0.980 | 0.204 | 5.38 |
| 20 | ss86333005 | 35266596 | CM | −0.765 | 0.179 | 4.36 |
| 20 | ss86306906 | 35610598 | CM | 0.778 | 0.181 | 4.41 |
| 20 | ss117972835 | 36202144 | CM | −0.863 | 0.177 | 5.55 |
| 20 | rs41938511 | 36232606 | CM | −0.984 | 0.193 | 6.02 |
| 20 | rs42542144 | 36520617 | CM | −0.880 | 0.191 | 5.02 |
| 20 | rs41940571 | 37740343 | CM | 1.127 | 0.193 | 7.79 |
| 20 | rs41947330 | 37946352 | CM | −0.879 | 0.179 | 5.64 |
| 20 | rs29018751 | 39518858 | CM | −0.884 | 0.204 | 4.45 |
| 20 | rs41581087 | 39556494 | CM | −0.823 | 0.187 | 4.60 |
| 20 | ss105263178 | 41861300 | CM | 0.969 | 0.195 | 5.75 |
| 20 | rs41641052 | 43585047 | CM | −0.798 | 0.176 | 4.82 |
| 20 | rs41641055 | 44311000 | CM | −0.970 | 0.215 | 4.82 |
| 20 | ss86292111 | 44333199 | CM | −0.922 | 0.215 | 4.36 |
| 23 | rs41600165 | 11692055 | CM | −0.690 | 0.154 | 4.74 |
| 25 | ss86306865 | 12503168 | CM2 | 0.221 | 0.053 | 4.22 |

### Discussion

The QTL intervals observed with association mapping were much narrower than those reported by Lund et al. (2008) who used linkage study with sparse map of microsatellite markers. Association mapping utilizes population level linkage disequilibrium. It therefore can map a QTL to a very small chromosomal region. The definitions of the mastitis traits were slightly different in Lund et al. (2008) and the present study. Thus, clinical mastitis for the first lactation (−10 to 305 d) was studied as one trait (CM1), while we have divided the first lactation mastitis into two sub-traits (CM11 and CM12). On BTA4, we detected a QTL affecting CM and SCS at 19.7 Mb. We further observed 3 SNPs between 66.46-66.61 Mb had −log₁₀(p) values between 3.5-3.8. We also detected two suggestive QTL for CM at 40.3 and 97.0 Mb on BTA5.

The strongest association of SNP to mastitis traits in this study was observed on BTA6 at 89.2 Mb. This QTL affected CM, CM11 and SCS. The most significant SNP, ss86341106, is located within the gene Deoxycytidine kinase (DCK), which catalyzes the rate-determining step in the deoxyribonucleoside salvage pathway. The highest levels of DCK expression are found in thymus and bone marrow, which indicates a role of DCK in lymphopoiesis. Indeed, knockout mice lacking enzyme activity revealed a combined immune deficiency phenotype, i.e. they produce very low levels of both T and B lymphocytes (Toy et al., 2010). Another strong candidate gene in this region is the IGJ gene, which encodes the immunoglobulin J polypeptide. This protein serves a nucleating function in the formation of the immunoglobulin M (IgM) pentameric complex and in the assembly of IgA dimers and polymers. IgM is the first antibody produced in the primary immune response to microbial infections and therefore plays a crucial role in preventing systemic spread of the pathogen (Racine and Winslow, 2009). Also IgA is engaged in the defense against microorganisms, in particular those that invade the host through mucosal surfaces. Thus, IgA is the major antibody class found in mucosal secretions, where it combines with microbes to prevent them from attaching to or penetrating the mucosal membranes (Lamm, 1997).

We have also detected another QTL at 25.2 on BTA6 significantly associated with the SNP ss61565956. An interesting candidate gene in this region is DAPP1, also known as Bam32, which is expressed in B cell lymphocytes and has been implicated in B cell antigen receptor (BCR) signaling. Thus, antigen binding to BCR involves a chain of signaling processes that are critical for B cell-fate decisions such as proliferation and differentiation, and BCR-mediated antigen internalization, processing, and presentation to T cells (Pierce, 2002). Studies of Bam32 deficient mice have shown that Bam32 mediates BCR-induced proliferation of B cell but not survival (Han et al., 2003), it regulates B cell antigen receptor internalization (Niiro et al., 2004), and it promotes the formation of stable interactions between B cells and T cells needed for efficient T cell activation, most likely by promoting adhesion to integrin ligands expressed on T cells (Al-Alwan et al., 2010).

We also found suggestive evidence for a QTL affecting CM at 75.9 Mb on BTA9.

We observed a suggestive evidence for a QTL affecting CM at 69.2 Mb on BTA11. Also, a suggestive QTL for CM was observed in our analysis at 30.4 Mb.

On BTA13, we detected a genome-wide significant QTL for CM, CM11 and CM12 at 57.7 Mb on BTA13. There were two closely located SNPs, which showed genome-wide association, located very close to the endothelin 3 gene [http://www.ensembl.org/Bos_taurus/]. The endothelins ET-1, ET-2, and ET-3 constitute a family of 21-amino acid peptides that are produced by numerous cells and tissues such as macrophages, and endothelial and epithelial cells (Giaid et al., 1991). In addition to a vasoconstrictive effect, they also have an impact on many different cell types, including activation of neutrophils (Elferink and De Koster, 1998). Neutrophils are blood-borne leukocytes that combat bacterial and fungal infections by phagocytosis or release of antimicrobial peptides (Selsted and Ouellette, 2005). Another possible candidate gene located in this region is Phactr3 (phosphatase and actin regulator 3), which has been shown to stimulate cell spreading and migration through direct interaction with the actin cytoskeleton (Sagara et al., 2009). Cell mobility is critically important for cell-mediated immune response (Luster et al., 2005). Lund et al. (2008) detected QTL for SCS between microsatellite markers BM9248 (29.1 cM) and BL1071 (68.6 cM; 71.9 Mb on Btau_{—}4.0). The QTL interval reported was very large (39.5 cM) in the linkage analysis. In contrast, the present GWAS was able to narrow the QTL to a 4 Mb region.

We have identified a genome-wide significant QTL at 37.7 Mb on BTA20. The most significant SNP, rs41940571 is linked to the gene RIPTOR independent companion of MTOR, complex 2. There are several other genes located in this QTL region in the Btau_{—}4.0 assembly. Among these is the C9 gene, encoding the complement component C9 precursor. The complement system is part of the immune response against invading pathogens. Activation of the complement system through the classical, alternative, or mannan-binding lectin pathways ultimately leads to formation of the Membrane Attack Complex, which creates pores in bacterial membranes, resulting in cell lysis. Complement C9 is the pore-forming subunit of MAC and mutations in this gene are associated with increased risk of infections, for example meningococcal meningitis (Kira et al., 1998; Zoppi et al., 1990; Horiuchi et al., 1998). Lund et al. (2008) observed a QTL for UD between 31.3 and 48.2 Mb on BTA20. These two studies point probably to the same QTL.

On BTA23, Lund et al. (2008) observed a QTL for SCS between BMS466 (46.1 cM; 43.4 Mb on Btau_{—}4.0) and INRA090 (53.2 cM) and a QTL for UD between 43.9-46.6 Mb. Ashwell et al. (1997) and Heyen et al. (1999) detected QTL for SCS on BTA3 at 39.9 and 48.6 Mb, respectively. Our study found suggestive evidence for a QTL affecting CM and SCS on this chromosome at 11.7 Mb, far away from the earlier reports. The QTL we found could be a different one than those reported earlier.

We also detected a genome-wide significant QTL affecting CM and CM2 at the proximal end of BTA14 (0.7 Mb) which was not detected in the same population by Lund et al. (2008). Three SNPs showed genome-wide significant association with MAS2. A region around 1.3 Mb with CYP11B1 harbors a QTL for SCS in German Holstein cattle (Kaupe et al. 2007) which could be the same QTL as detected in the present study. There are several genes located in the QTL region in Btau_{—}4.0 including DGAT1 (Grisart et al. 2002) which has a large influence on phenotypic variance in milk fat content and other milk characteristics.

The genetic correlation between clinical mastitis and SCS is >0.70 (Lund et al. 1999, Carlen et al. 2004; Heringstad et al. 2006). Therefore, it was expected that many of the QTL affecting CM would also affect SCS. Out of nine significant QTL affecting clinical mastitis traits, five showed effect on SCS. This was as expected due to high genetic correlation between clinical mastitis and SCS. As we are analyzing both clinical mastitis and SCS in the present study, may help to indicate the extent of SCS QTL from the literature can be expected to affect clinical mastitis. Out of the six mastitis traits analyzed in the present study, maximum number of QTL was observed for mastitis index which was an index combing clinical mastitis from first three lactations.

The present study identified several mastitis QTLs. We used association study with dense SNP markers in a mixed model analyses which was observed to perform best for samples from complex pedigreed population like cattle (Sahana et al. 2010b). In the present study QTL positions were refined to much narrower genomic regions than has been possible by previous linkage analysis. This association mapping identified SNPs which are in linkage disequilibrium with the QTL, or which are causative mutations, and therefore, marker-based selection at the population level for mastitis resistance could be carried out. Some of the QTL regions were narrow enough to initiate further search for candidate genes underlying mastitis QTL.

### Example 2

### Ultra-Fine-Mapping of Clinical Mastitis and Somatic Cell Score QTL in Dairy Cattle

Clinical mastitis and somatic cell score QTL in dairy cattle were fine-mapped using high-density SNP Chips comprising 777,962 SNP probes.

### Association Mapping

Association mapping identifies specific functional variants (i.e., loci, alleles) linked to phenotypic differences in a trait, to facilitate detection of trait causing DNA sequence polymorphisms and/or selection of genotypes that closely resemble the phenotype. Association mapping has been variously defined (Chakraborty and Weiss 1988; Kruglyak 1999), and has also been referred to as “association genetics,” “association studies,” and “linkage disequilibrium mapping”. Genome-wide association studies (GWAS) provide an important avenue for undertaking an agnostic evaluation of the association between common genetic variants and risk of disease or quantitative traits. Recent advances in our understanding of genetic variation and the technology to measure such variation have made GWAS feasible.

In the present example, association mapping has been used to identify single nucleotide polymorphisms (SNPs) which are associated with mastitis resistance in dairy cattle. Several Quantitative Trait Loci (QTL) were identified which can be usefully applied in selection of animals for improvement of resistance to mastitis.

### Phenotypes

The genome scan for mastitis resistance was carried out using Danish and Swedish Holstein cattle for nine mastitis phenotypes analysed. The phenotype used for mapping quantitative trait loci (QTL) for mastitis resistance was udder health index estimated for Nordic cattle genetic evaluation (NAV, Pedersen, 2008, www.nordicebv.info). The udder health traits currently evaluated in NAV included four clinical mastitis traits from three lactations, all measured as a binary trait (Table 5). These four mastitis traits are weighted together to form a mastitis resistance index (CM), standardized to a mean of 100 and a standard deviation of 10 (Johansson et al. 2007). There were 4200 progeny tested bulls from Danish, Swedish and Finnish Holstein dairy cattle with recode for these nine mastitis related phenotypes. The SNP genotype and phenotypes of these bulls were utilized for association mapping.

TABLE 5
Abbreviations and definitions of traits included in the study

| Trait | Trait | |
|---|---|---|
| No. | abbreviation | Trait definitions |
| 1 | CM11 | Clinical mastitis (1) or not (0) between −15 and |
| | | 50 days after 1st calving |
| 2 | CM12 | Clinical mastitis (1) or not (0) between 51 and |
| | | 305 days after 1st calving |
| 3 | CM2 | Clinical mastitis (1) or not (0) between −15 and |
| | | 305 days after 2nd calving |
| 4 | CM3 | Clinical mastitis (1) or not (0) between −15 and |
| | | 305 days after 3rd calving |
| 5 | CM | Clinical mastitis: 0.25*CM11 + 0.25*CM12 + |
| | | 0.3*CM2 + 0.2*CM3 |
| 6 | SCC1 | Log. somatic cell count average in 1st lactation |
| 7 | SCC2 | Log. somatic cell count average in 2nd lactation |
| 8 | SCC3 | Log. somatic cell count average in 3rd lactation |
| 9 | SCC | Log somatic cell count: 0.5*SCC1 + 0.3*SCC2 + |
| | | 0.2*SCC3 |

### Genotypes

The Holstein bulls were genotyped using the Illumina Bovine SNP50 BeadChip. Genotyping was done by the Illumina Bovine SNP50 BeadChip (Illumina Inc., http://www.illumina.com/Documents/products/datasheets/datasheet_bovine_snp5O.pdf) at the Danish Institute of Agricultural Sciences, Research Center Foulum, Department of Molecular Biology and Genetics and at GenoSkan, AgroBusiness Park Foulum. The platform used was an Illumina® Infinium II Multisample assay device. SNP chips were scanned using iScan and analyzed using Beadstudio ver. 3.1 software. The quality parameters used for selection of SNPs were minimum call rates of 85% for individuals and of 95% for loci. Marker loci with minor allele frequencies (MAFs) below 5% were excluded. The minimal acceptable GC score was 0.60 for individual typings. Individuals with average GC scores below 0.65 were excluded. The number of SNPs after quality control was 43,415 in the 50 k dataset. A total of 557 Holstein bulls in the EuroGenomics project (Lund et al., 2011) were regenotyped using the BovineHD Genotyping BeadChip (http://www.illumina.com/Documents/products/datasheets/datasheet_bovineHD.pdf). There are a total of 777,962 SNPs on the BovineHD BeadChip that uniformly span over entire bovine genome with an average gap size of 3.43 kb and a median gap size of 2.68 kb. The quality control parameters set for HD data were similar as it was for 50K chip as described above. The 50 k genotypes were imputed to the HD genotypes using Beagle software package (Browning and Browning, 2009), based on the marker data of the HD genotyped bulls (Su et al 2011; interbull meeting presentation). The markers in the 50 k chip but not included in the HD chip were excluded in the imputation process. The number of SNPs after imputation to BovineHD chip was 648,219. The genome positions of the SNPs were taken from UMD3.1 assembly (http://www.ensembl.org/Bos_taurus/2011_{—}09_cow_genebuild.pdf). The physical maps for the 648,219 SNPs located on 29 Bovine autosomes are available at www.illumina.com.

### The Model Used for Association Mapping

The details of the association mapping model are described by Yu et al. (2006) and Sahana et al. (2010). The statistical model used for association analyses was:

yᵢ=μ+bxᵢ+sᵢ+eᵢ

Where yᵢ was the single trait estimated breeding value of individual i, μ was the general mean, xᵢ was a count in individual i of one of the two alleles (with an arbitrary labeling), b was the allele substitution effect, sᵢ was the random effect of the sire of individual i, assumed to have a normal distribution N(0, Aσₛ²), where A is the additive relationship matrix and σₛ² is the sire variance, and eᵢ was a random residual of individual i assumed to follow a normal distribution with mean zero and error variance, σₑ². Testing was done using a Wald test against a null hypothesis of H₀: b=0. The significance threshold was determined using a Bonferroni correction. The genome-wide significance threshold was calculated by dividing the nominal significance threshold of 0.05 by the total numbers of SNPs included in the analysis.

### Results

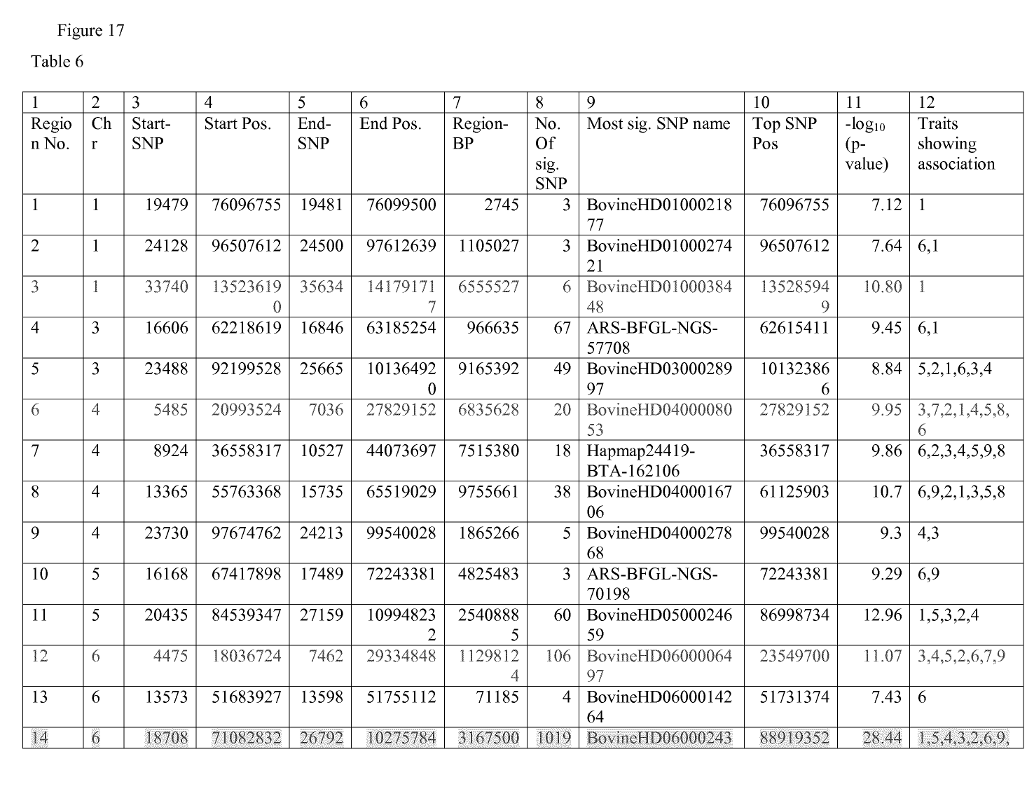

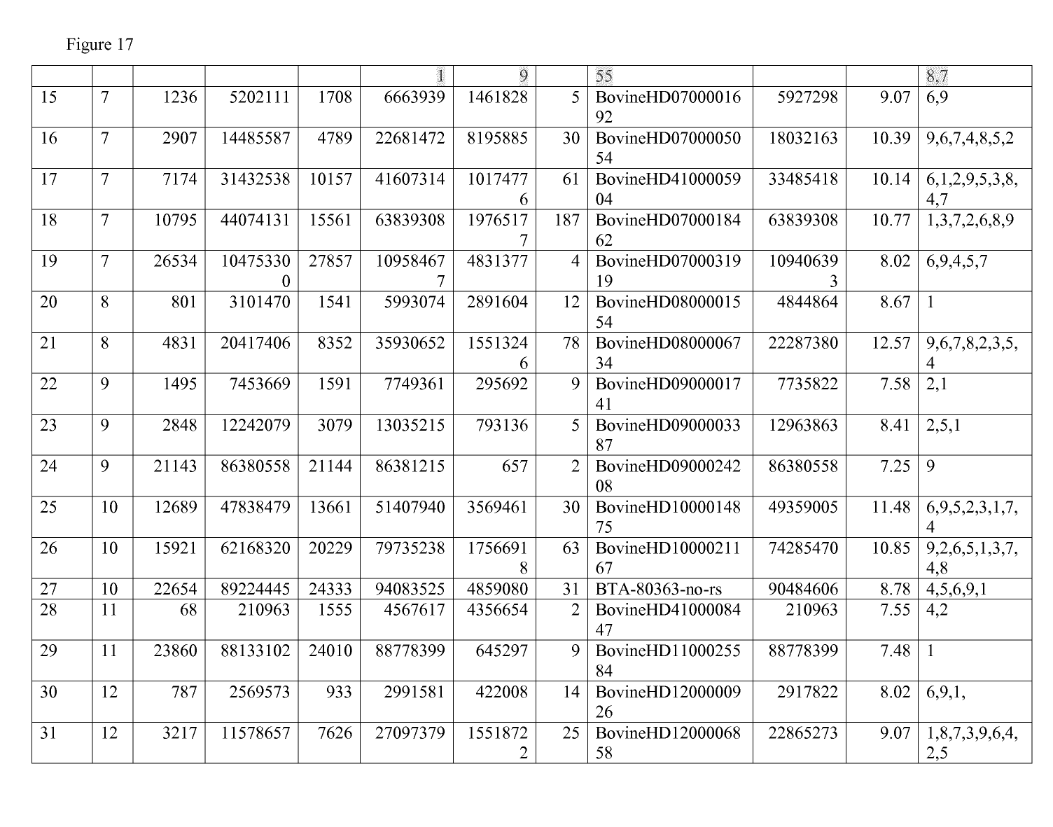

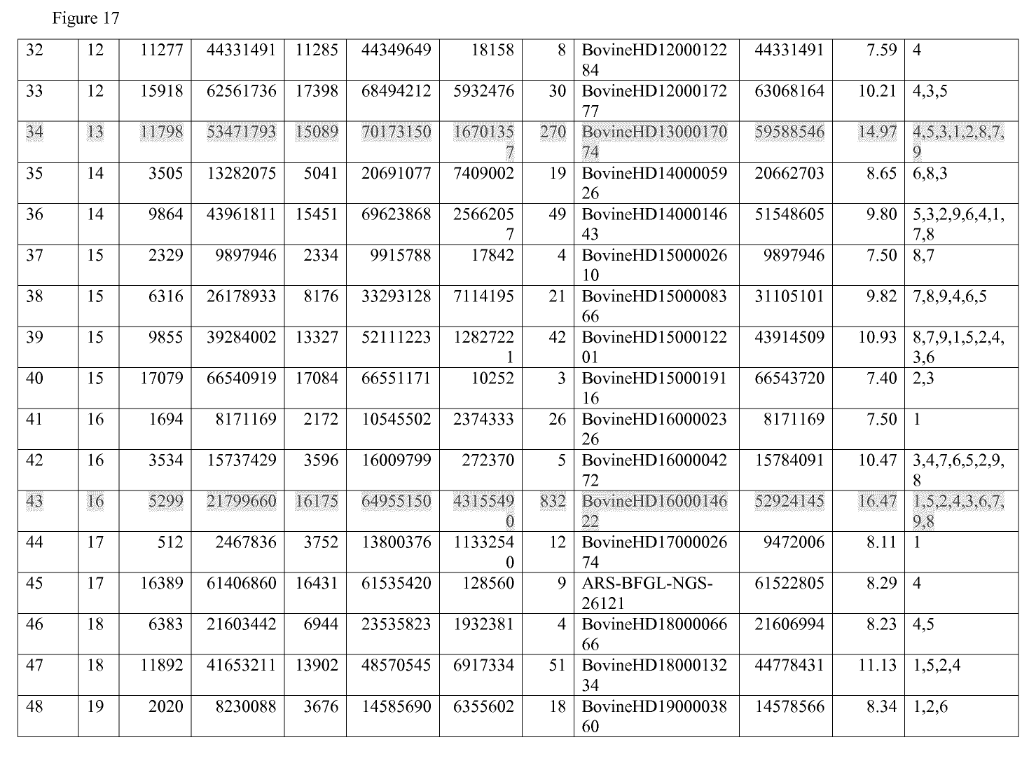

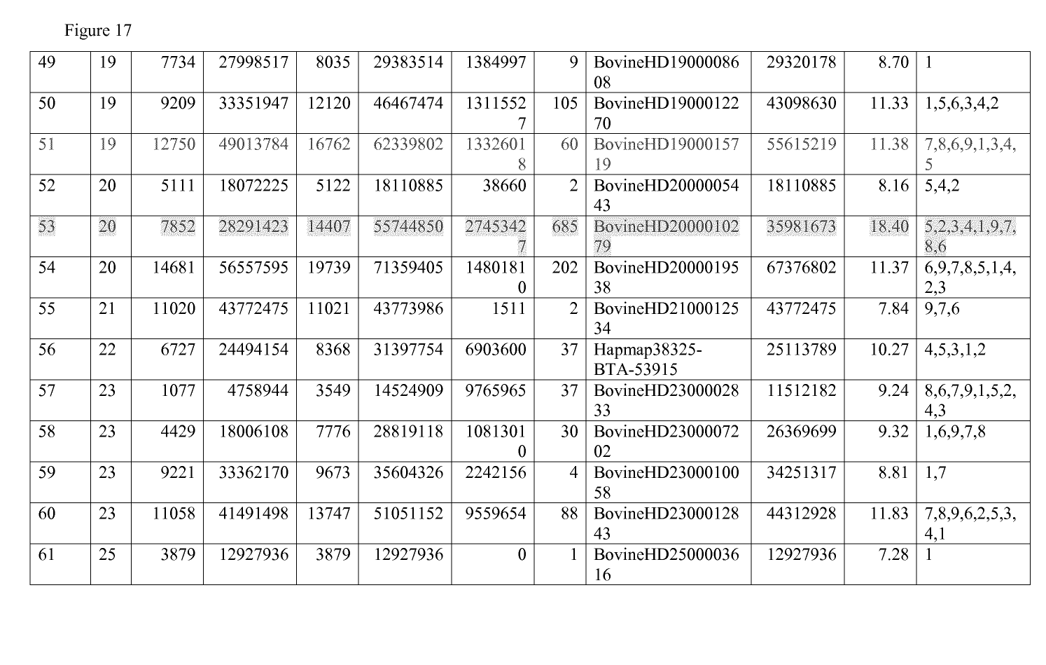

A total 61 QTL regions on 22 chromosomes associated with mastitis related traits were identified. The QTL regions along with the highest significantly associated SNP for each QTL are presented in Table 6; cf. FIG. 17. The data sheet for the BovineHD Genotyping BeadChip can be downloaded from: (http://www.illumina.com/Documents/products/datasheets/datasheet_bovineHD.pdf). The names and positions of the SNPs are available the website of Illumina, cf. www.Illumina.com.

Table 6: cf. FIG. 17

TABLE 7

| Column | Column | |
|---|---|---|
| number | headings | Description |
| 1 | Region No. | Serial number for the QTL regions |
| 2 | Chr | Chromosome number |
| 3 | Start-SNP | The genome-wide significant SNP number |
| | | at the beginning of the QTL region |
| 4 | Start Pos. | Position of the ‘Start-SNP’ on the chromosome |
| | | (Bp) |
| 5 | End-SNP | The genome-wide significant SNP number |
| | | at the end of the QTL region |
| 6 | End Pos. | Position of the ‘End-SNP’ on the chromosome |
| | | (Bp) |
| 7 | Region-BP | The QTL region in Bp |
| 8 | No. of sig. | Number of genome-wide significant SNP |
| | SNP | within the QTL region |
| 9 | Most sig. | The highest significant SNP within a QTL |
| | SNP name | region |
| 10 | Top SNP Pos | The highest significant SNP's position on |
| | | the chromosome in Bp |
| 11 | −log10(p- | −log₁₀(p-value) for the highest significant SNP |
| | value) | in the QTL region. |
| 12 | Traits showing | 1-CM11, 2-CM12, 3-CM2, 4-CM3, 5-CM, |
| | association | 6-SCC1, 7-SCC2, 8-SCC3, 9-SCC. The |
| | | descriptions of the traits are given in the text. |

### Example 3

### Targeted Genome-Wide Association for Causative Mutation Using Whole Genome Sequence Data for a QTL Region on BTA6 (88-96 Mb)

### Targeted Region (TR).

The genomic region from 88-96 Mb on BTA6 was selected for targeted genome-wide association study with SNP variants identified from the whole genome sequence of 90 bulls. This genomic region was selected as it showed the strongest association with clinical mastitis in analyses of the Illumina Bovine SNP50 BeadChip (HD SNP chip). The most significant SNP association with clinical mastitis for HD SNP chip analyses was BovineHD0600024355 located at 88,919,352 Bp on BTA6.

### Whole Genome Sequence (WGS).

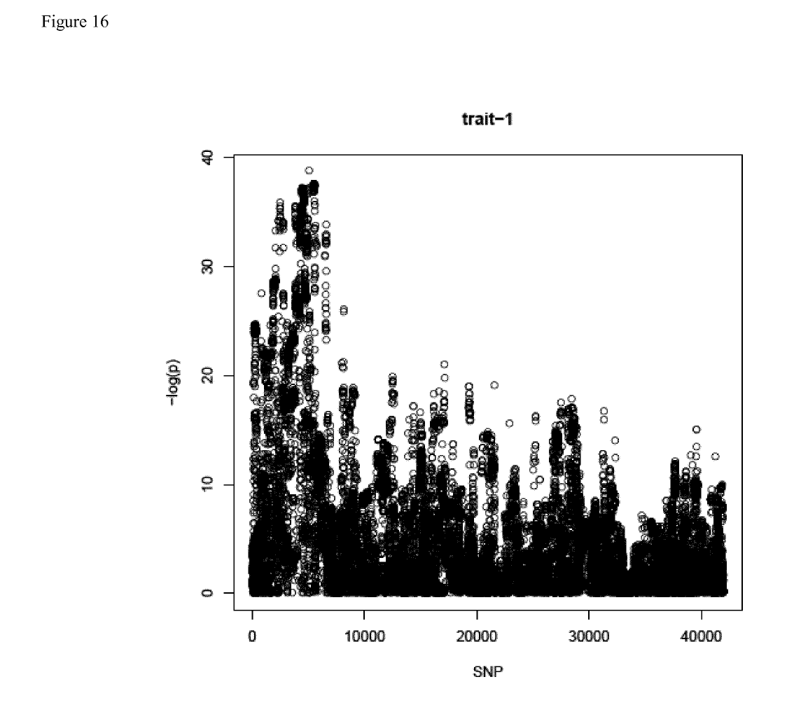

The whole genome of ninety bulls from three breeds (˜30 each from Nordic Holstein, Danish Jersey and Nordic Red breed) was sequenced (˜10× coverage) at Beijing Genomic Institute (BGI), China. The whole genome sequences were analyzed and more than 24 million variants were observed. The variants were functionally annotated. The SNP polymorphisms for the targeted region (TR) on BTA6 harbouring mastitis QTL were extracted. There were a total of 41,993 SNP variants within the TR of 8 Mb. There were 5,193 Nordic Holstein bulls with the clinical mastitis phenotypes and the HD SNP chip genotypes. These animals were imputed for the 41,993 SNP variants identified in WGS using software Beagle (Browning and Browning, 2007). The association analyses were carried out for these 5,193 bulls' data using the mixed linear model analysis (Yu et al. 2006). The results showed an association with clinical mastitis of the neuropeptide FF receptor 2 (NPFFR2) gene (FIG. 16). The gene is located at 89,052,210-89,059,348 Bp on BTA6. A non-synonymous mutation within NPFFR2 gene, identified by SNP chr6_{—}89059253, located at 89,059,253 Bp on BTA6 had the −log 10(p-value)=37.4. This SNP variant is associated with clinical mastitis in the first lactation (CM11). Thus, the NPFFR2 gene appears to strongly affect clinical mastitis and the chr6_{—}89059253 SNP is likely the causative mutation affecting resistance to clinical mastitis in Nordic Holstein cattle or this SNP is in strong linkage disequilibrium with causative polymorphism responsible for resistance to clinical mastitis.

### Example 4

### Targeted Region-Wise Association Studies (RWAS)

Genome-wide association studies (GWAS) was carried out previously for nine mastitis traits in Nordic Holstein cattle. The genotyping was done using Bovine HD SNP chip. A linear mixed model analyses was carried out to identify the SNPs significantly associated with mastitis resistance. Based on this GWAS study, six genomic regions were selected for targeted GWAS with whole genome sequence data (Table 8).

### Whole Genome Sequencing

A total of 90 bulls' (˜30 of each of Danish Red, Danish Jersey and Nordic Holstein) whole genomes were sequenced at BGI, China. The sequence data was analyzed at by the Quantitative Genetics and Genomic Centre (QGG), MBG, Aarhus University. The average genome coverage was more than 10×. Alignment of sequence reads to the cattle reference genome was done and the candidate sites or regions at which one or more samples differ from the reference sequence were identified. The quality control measures removed candidate sites that likely were false positives. The variants calls i.e. the estimation of the alleles present in each individual at variant sites was carried out using VCF tools (http://vcftools.sourceforge.net/). A total of more than 24 millions DNA level variants (single nucleotide polymorphism (SNP), insertion-deletions (indel), copy number variation (CNV) etc.) observed across three cattle breeds. All the variants were functionally annotated for search of candidate polymorphisms affecting mastitis related traits.

### Targeted Imputation

Six chromosomal regions (Table 8) were selected based on GWAS study with HD SNP chip on nine mastitis resistance traits in Nordic Holstein cattle. The length of the regions and the number of SNP variants (from whole genome sequence data) for each region selected after quality control are given in Table 8. The phenotypes (estimated breeding values) were available for 5193 Nordic Holstein bulls for nine mastitis related traits. The SNP chip genotypes (50 k and 777 k) of these bulls were imputed to the sequence level for the targeted regions using the software Beagle (Browing and Browing, 2006). All the SNP position mentioned here is as per the Bovine genome assembly (UMD3.1).

TABLE 8
The selected targeted regions on six chromosomes for RWAS. The highest significant SNP
across nine mastitis traits analyzed for each targeted region is also presented in the table.

| | | | Trait with | | | | |
|---|---|---|---|---|---|---|---|
| | Region | No. of | lowest | | Position | | -log₁₀(p- |
| Chromosome | (Mb) | SNPs | p-value | SNP | (Bp) | MAF | value) |
| BTA5 | 84-95 | 55,046 | CM11 | Chr5_92753829 | 92,753,829 | 0.204 | 9.89 |
| BTA6 | 88-96 | 41,993 | CM11 | Chr6_88977023 | 88,977,023 | 0.432 | 38.76 |
| BTA13 | 57-63 | 18,935 | CM | Chr13_57608628 | 57,608,628 | 0.305 | 15.07 |
| BTA16 | 48-55 | 27,709 | CM11 | Chr16_50529178 | 50,529,178 | 0.019 | 14.51 |
| BTA19 | 55-58 | 16,145 | SCS3 | Chr19_55296191 | 55,296,191 | 0.380 | 10.90 |
| BTA20 | 32-40 | 30,025 | CM2 | Chr20_35965955 | 35,965,955 | 0.203 | 15.24 |

### Region-Wise Association Studies (RWAS)

A SNP-by-SNP analysis where each SNP was fitted separately in a linear mixed model (LMM) following Yu et al. (2006). Complex familial relationship is the primary confounding factor in GWAS study in livestock population. LMM which include the relationship among individuals through a polygenic effect is able to control the false positives due to family structure (Yu et al., 2006).

### Linear Mixed Model

For each SNP separately, the association between the SNP and the phenotype was assessed by a single-locus regression analysis using a linear mixed model. The model was as follows:

y=1μ+mg+Zu+e

where y is the vector phenotypes (EBV), 1 is a vector of 1 s with length equal to number of observations, p is the general mean, m is the genotypic score (obtained from Beagle output; values ranged between 0 and 2) associating records to the marker effect, g is a scalar of the associated additive effect of the SNP, Z is an incidence matrix relating phenotypes to the corresponding random polygenic effect, u is a vector of the random polygenic effect with the normal distribution N(0, Aσᵤ²), where A is the additive relationship matrix and σᵤ² is the polygenic variance, and e is a vector of random environmental deviates with the normal distribution N(0, Aσₑ²), where σₑ² is the error variance. The model was fitted by restricted maximum likelihood (REML) using the software DMU (Madsen and Jensen, 2011) and testing was done using a Wald test against a null hypothesis of g=0.

### Significant Associations

A SNP was considered to have significant association if the p-value crossed the region-wise significant threshold after Bonferroni correction for multiple testing.

Association Analyses with the Most Important SNP as Cofactor in the Model

A large number of SNPs crossed region-wide significant threshold. As the LD is expected to be high these significant effect of the SNPs could be due to linkage to only one casual variant segregating in the targeted region. However, as the regions were quite large (>5 Mb in some cases), it is also possible that the effect observed was due to multiple causative variants segregating in Nordic Holstein population. This analysis was done to see if any SNP shows significant association after the most important SNP from the LMM analyses and/or functional annotation was included in the model as cofactor (Table 9). The analysis was done using lme function of nlme of R-package (http://cran.r-project.org/). The model was as below.

Yᵢⱼ=μ+Sᵢ+fixSNP+SNPₘ+eᵢⱼ

where Yᵢⱼ is the residual phenotype obtained from an animal model (i.e. adjusted for the pedigree) for the jth animal of ith sire, Si is the random effect of the i^{th} sire, fixSNP is the regression of genotype score for the highest significant SNP from the LMM (or the most important SNP based on functional annotation among a few top ones), SNPₘ is the regression of the genotype score of the m^{th} SNP (m≠fixSNP) and eᵢⱼ is the random error.

TABLE 9
The SNP selected based on the strength of association and also
functional annotation to be used as cofactor the linear model.

| Chromo- | Region | SNP used | SNP Position | |
|---|---|---|---|---|
| some | (Mb) | as cofactor | (Bp) | MAF |
| BTA5 | 84-95 | Chr5_92753829 | 92,753,829 | 0.204 |
| BTA6 | 88-96 | Chr6_89059253 | 89,059,253 | 0.483 |
| BTA13 | 57-63 | Chr13_57572723 | 57,572,723 | 0.137 |
| BTA16 | 48-55 | Chr16_50529178 | 50,529,178 | 0.019 |
| BTA19 | 55-58 | Chr19_55296191 | 55,296,191 | 0.380 |
| BTA20 | 32-40 | Chr20_35965955 | 35,965,955 | 0.203 |

### Results

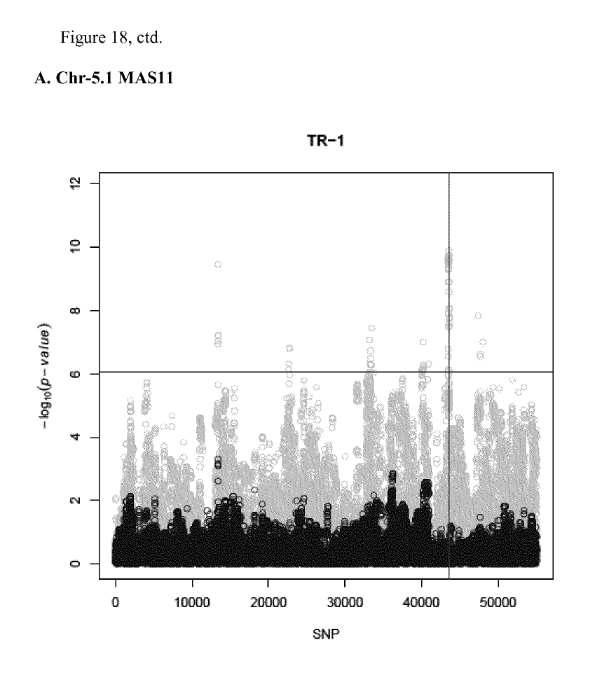

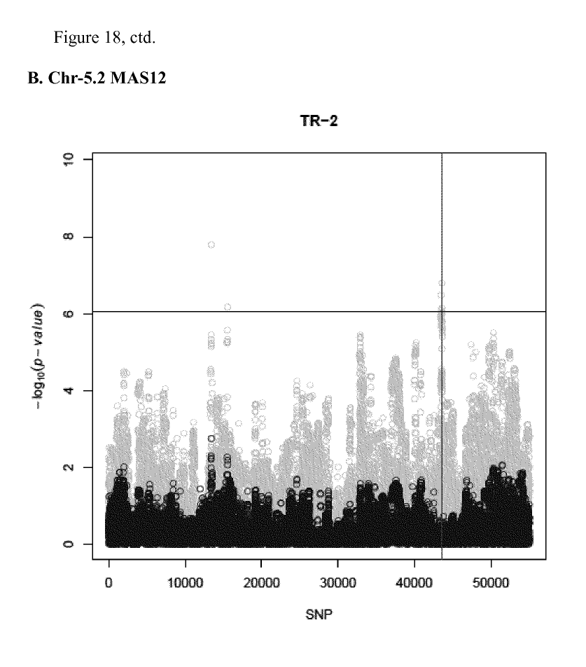

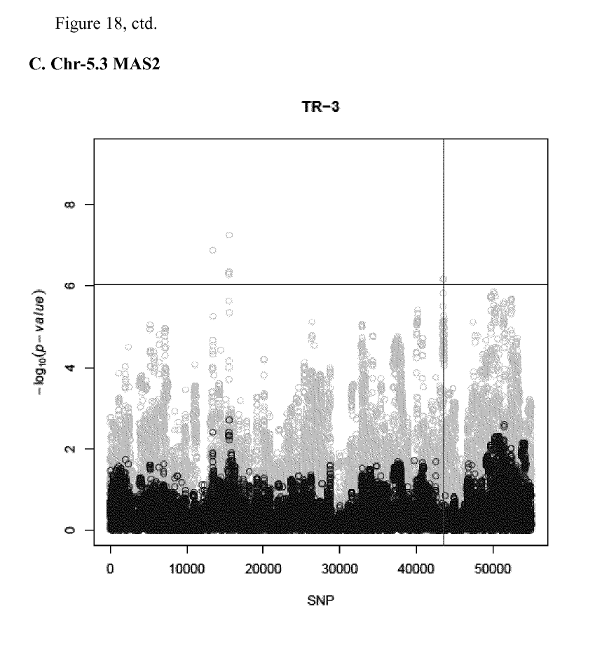

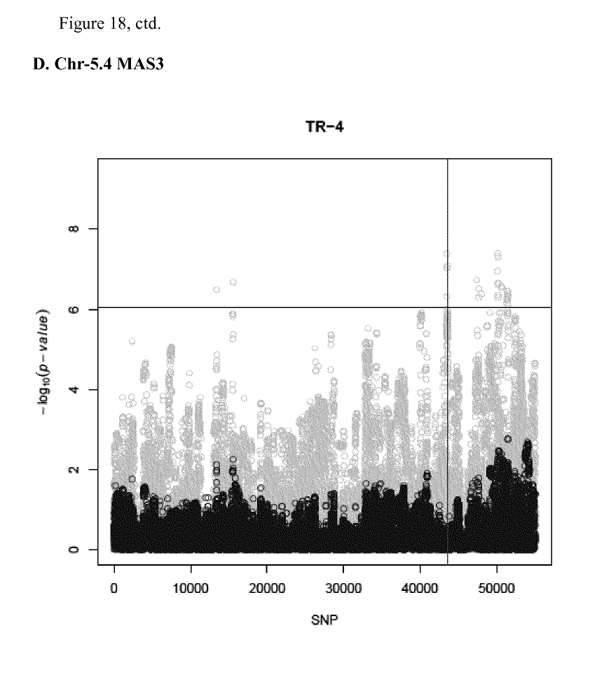

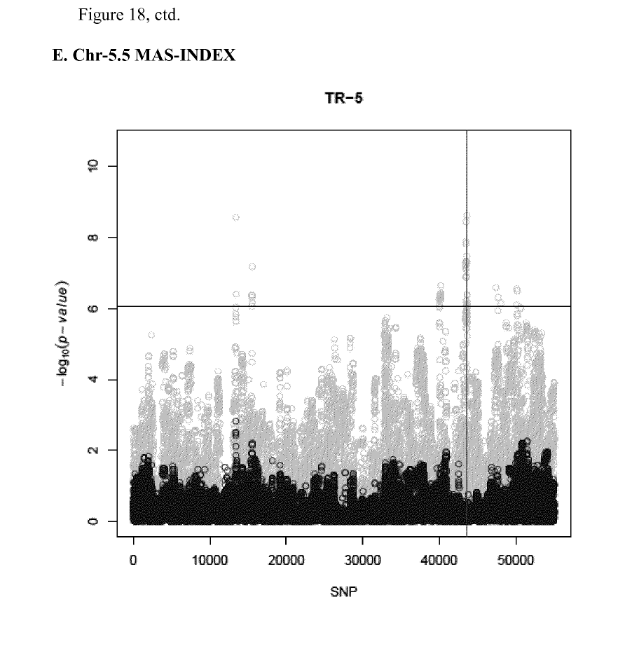

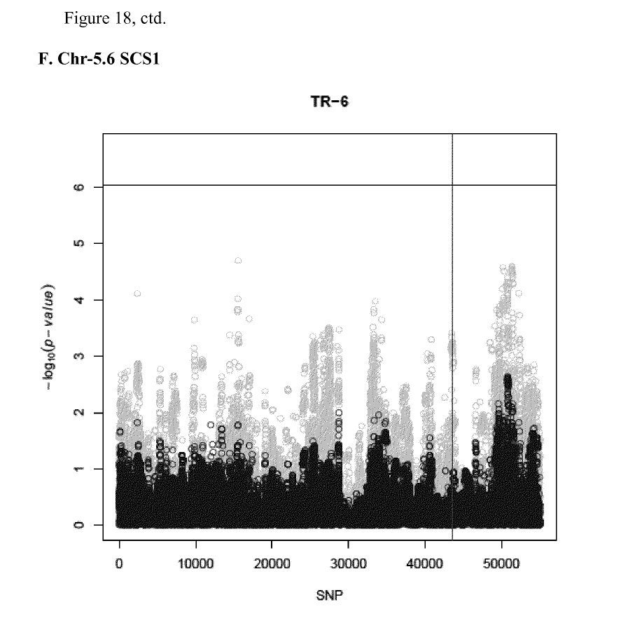

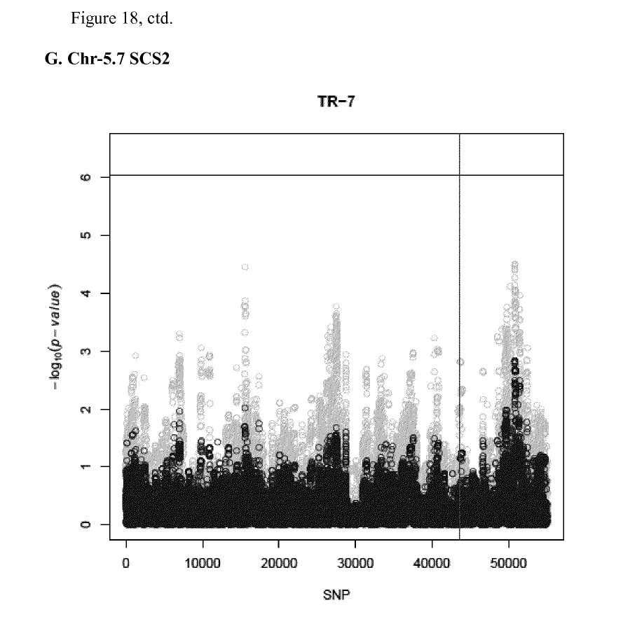

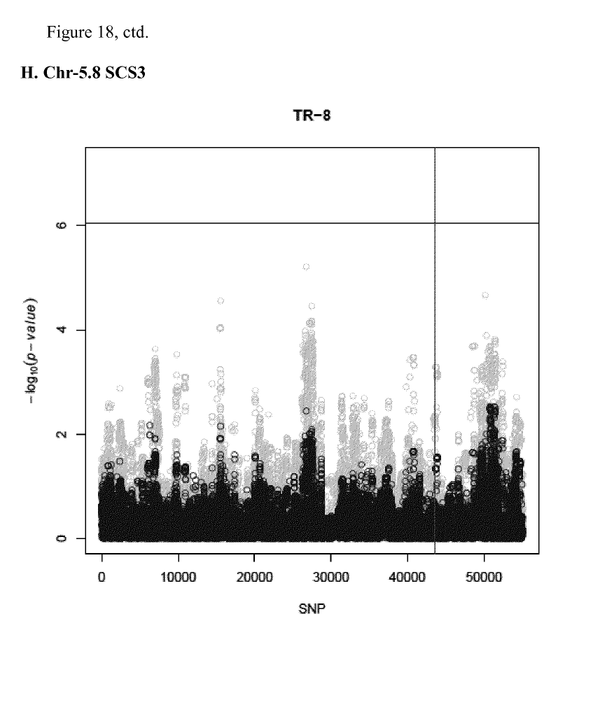

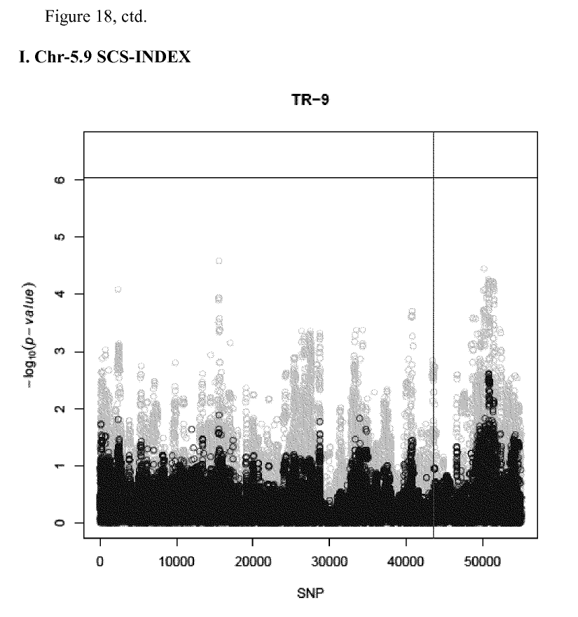

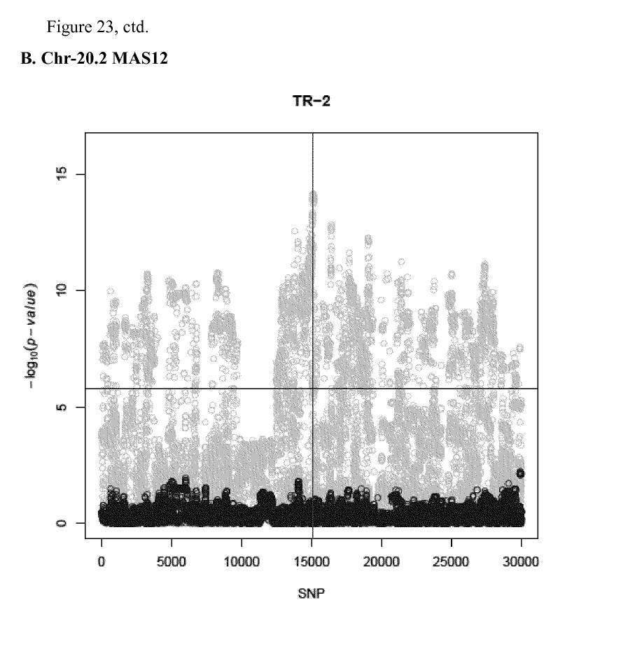

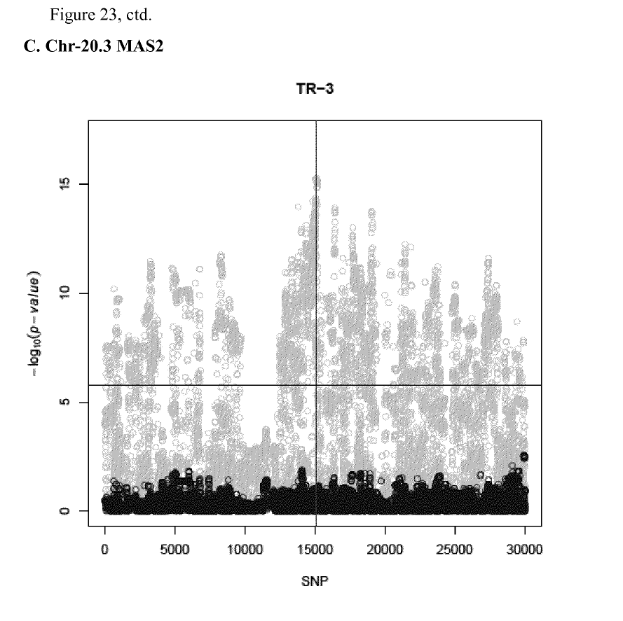

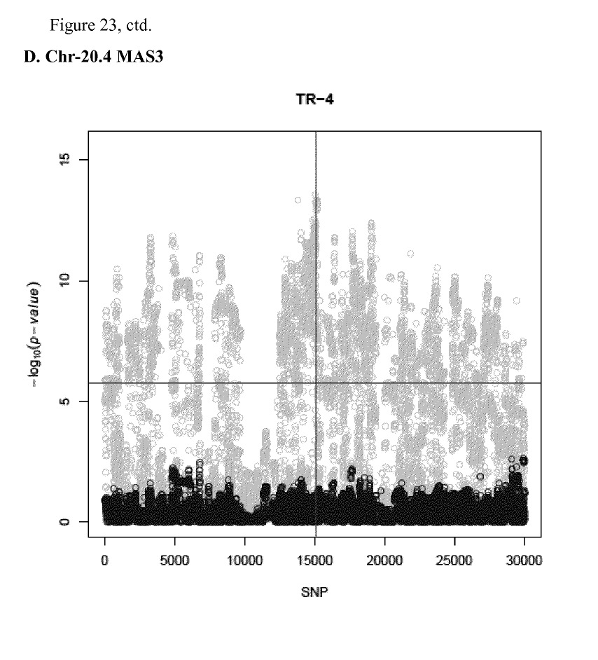

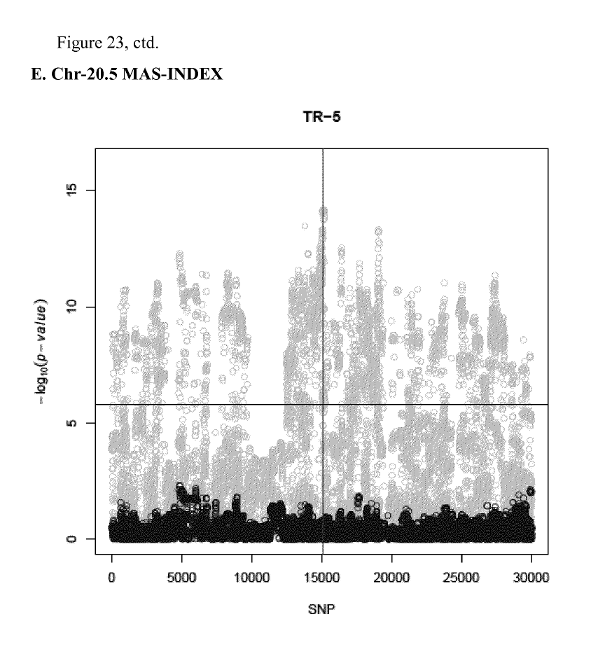

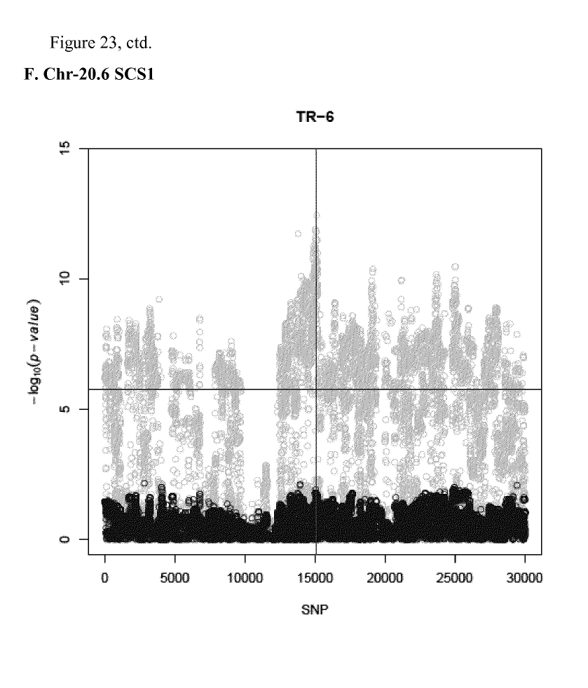

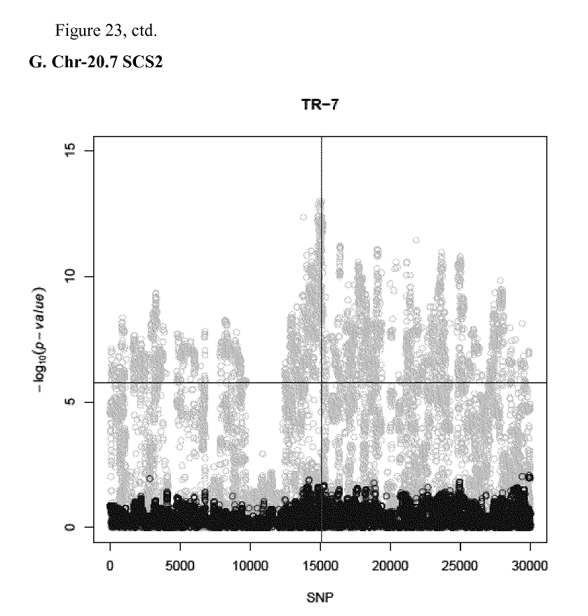

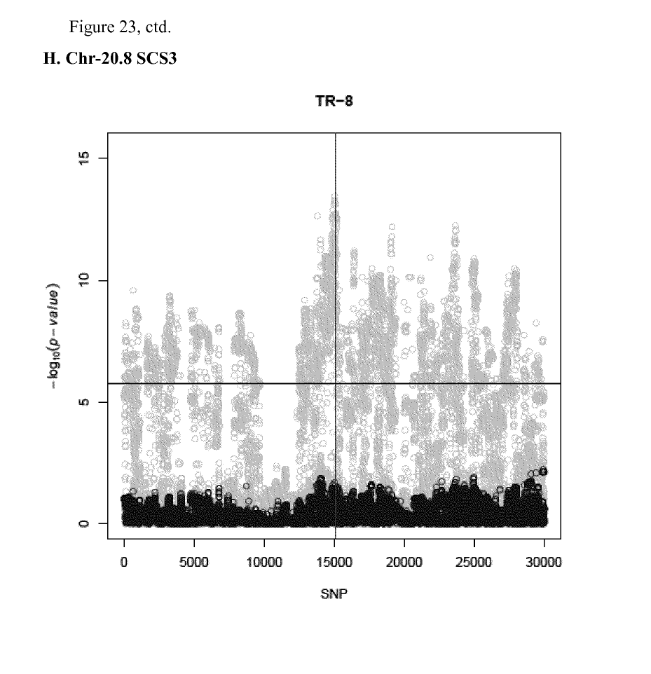

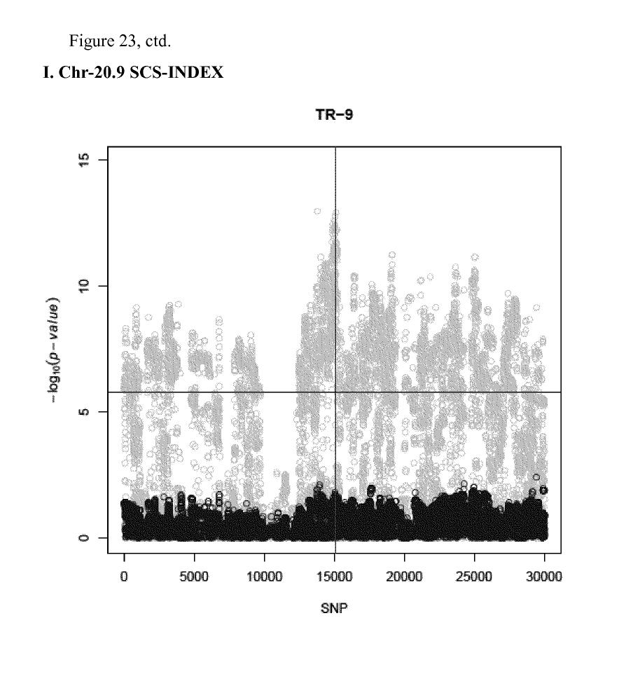

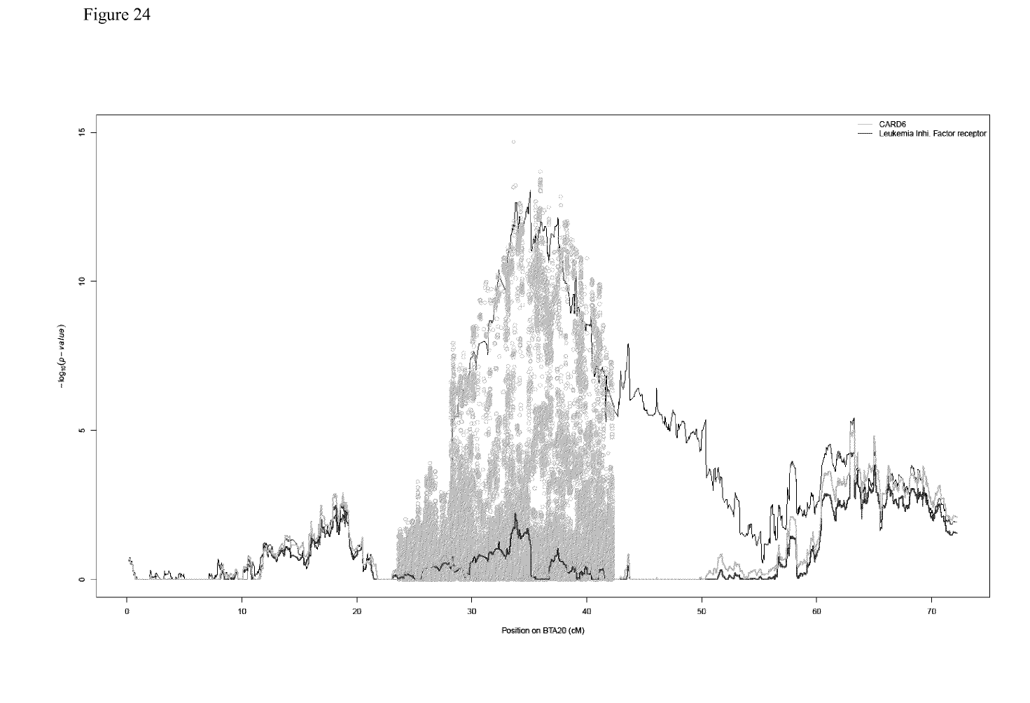

The manhattan plots for the RWAS (both linear mixed model, and the linear model with the most important SNP as cofactor) are presented in FIGS. 18-23. The lists of the most significant SNP associated with nine mastitis traits in Nordic Holstein cattle for each of these genomic regions selected for targeted GWAS are presented in the tables below. The candidate polymorphisms of the each of the targeted regions were searched based on the functional annotation information and examined for their association strengths.

All the six targeted regions had wide picks of association. However, including the most significant associated SNP as cofactor (Table 9), the entire range of associated region collapses. This indicates the SNPs mentioned in Table 9 which were used as cofactor are either the real causal polymorphisms affecting mastitis resistance in Nordic Holstein or are in very high LD with the real causal polymorphism in the targeted regions. Therefore, these SNPs could be used as predictor of mastitis resistance on individual animals in Holstein cattle. Results from individual genomic regions are discussed in details below.

### BTA5 (84-95 Mb)

The most significant SNP for each of the nine mastitis related traits are presented in table 10 for the targeted region on BTA5. The total length of the targeted region on BTA5 was 9 Mb and there were two regions (at 86.99 and 92.75 Mb) where the highly significant SNPs were concentrated. The manhatton plot for this region is presented in FIG. 18.

TABLE 10
The most significant SNP association for nine mastitis traits in the targeted region on BTA5

| | | | | | | | | Allele |
|---|---|---|---|---|---|---|---|---|
| | | SNP | | | | | | increasing |
| | | position | | | | -log₁₀(p- | | mastitis |
| Trait | SNP name | (Bp) | MAF | b-value | SE | value) | Genotype | resistance |
| CM11 | Chr5_92753829 | 92753829 | 0.204 | 2.042 | 0.317 | 9.89 | A/G | G |
| CM12 | BovineHD0500024659 | 86998734 | 0.487 | −1.135 | 0.201 | 7.80 | G/A | G |
| CM2 | Chr5_87360522 | 87360522 | 0.222 | 14.056 | 2.582 | 7.26 | A/T | T |
| CM3 | BovineHD0500026657 | 93941017 | 0.254 | −1.224 | 0.223 | 7.40 | A/G | A |
| CM | Chr5_92753829 | 92753829 | 0.204 | 1.869 | 0.313 | 8.61 | A/G | G |
| SCS1 | Chr5_87360522 | 87360522 | 0.222 | 11.068 | 2.593 | 4.70 | A/T | T |
| SCS2 | Chr5_94040670 | 94040670 | 0.160 | −1.577 | 0.378 | 4.51 | C/A | C |
| SCS3 | Chr5_89528205 | 89528205 | 0.020 | 13.344 | 2.947 | 5.22 | G/T | T |
| SCS | Chr5_87360522 | 87360522 | 0.222 | 10.758 | 2.556 | 4.58 | A/T | T |

Candidate Polymorphism within the BTA5 Targeted Region:

BTA5 (86.99 Mb): There is a huge intron at 86.99 Mb. Upstream there is a non-synonymous polymorphism (allele frequency of the alternative allele for the polymorphisms (alt) 64%) at 86,948,388 which could be the candidate polymorphism. Downstream at 87,004,771 (alt 15%) and 87,004,957 (alt 3%), there are two polymorphisms in a non-coding gene in an intron. Further downstream there is a synonymous coding splice-site polymorphism at 87,023,448 (alt 31%).

BTA5 (92.75 Mb): The gene around 92,496,500 has three polymorphisms at 92,496,251 (alt 54%), 92,496,510 (alt 28%) and 92,496,586 (alt 3%). Downstream the next annotation starts around 93,688,996 (gene ENSBTAG00000013541). However, all polymorphisms in this gene are either intronic, upstream or downstream. The next downstream a candidate causative polymorphism could be at 93,939,231 (alt 7%) (gene ENSBTAG00000008541) which is non-synonymous coding. However, none of above candidate polymorphisms discussed within the targeted region of BTA5 showed strong association signal across the mastitis traits analyzed.

BTA5: Genes associated with mastitis according to the analysis are summarized in the table below. For clinical mastitis the top SNPs are concentrated around 92.7 Mb, whereas there are minor peaks at positions 87 Mb, (88.8 Mb, 90.9 MB) and 93.4 Mb. The following genes are located in the two major peak regions around 87 Mb and 92.7 Mb.

TABLE 11
BTA5: Genes associated with mastitis according to the present analysis.

| | | Associated | Gene location |
|---|---|---|---|
| Ensembl Gene ID | Common gene name | gene name | (UMD3.1) |
| ENSBTAG00000022360 | Transcription factor SOX-5 | SOX5 | 86,571,273-87,036,285 |
| ENSBTAG00000005833 | Ethanolamine kinase 1 | ETNK1 | 87,967,760-88,017,062 |
| ENSBTAG00000001673 | Hypothetical protein | LOC520387 | 88,099,588-88,191,001 |
| | LOC520387 | | |
| ENSBTAG00000013202 | 1-phosphatidylinositol-4,5- | PLCZ1 | 91,771,436-91,820,146 |
| | bisphosphate phos- | | |
| | phodiesterase zeta-1 | | |
| ENSBTAG00000047048 | Novel_gene | | 91,880,701-91,882,214 |
| ENSBTAG00000046178 | Noncoding | | 91,945,426-91,946,169 |
| ENSBTAG00000020715 | Phosphoinositide-3-kinase, | PIK3C2G | 91,835,146-92,276,939 |
| | class 2, gamma polypeptide | | |
| ENSBTAG00000030493 | Ras-related and estrogen- | RERGL | 92,432,331-92,442,968 |
| | regulated growth inhibitor- | | |
| | like protein | | |
| ENSBTAG00000013541 | LIM domain only protein 3 | LMO3 | 93,693,961-93,757,644 |
| ENSBTAG00000008541 | Microsomal glutathione S- | MGST1 | 93,926,791-93,950,162 |
| | transferase 1 | | |
| ENSBTAG00000009444 | Solute carrier family 15, | SLC15A5 | 94,030,765-94,127,585 |
| | member 5 | | |

Among the candidate genes in this region we find RERGL encoding Ras-related and estrogenregulated growth inhibitor-like protein. There is little or no functional information about this specific gene in the literature. However, the Ras family of small GTPases is a group of more than 150 proteins that function in diverse biological processes including immunity and inflammation (Johnson and Chen, Current Opinion in Pharmacology 12, 458-463, 2012). Another good candidate gene which might be relevant in relation to mastitis is PIK3C2G, which codes for phosphoinositide-3-kinase class 2 gamma subunit. Many PI3K enzymes play an important role in the functioning of immune cells (Johnson and Chen, Current Opinion in Pharmacology, 2012; Koyasu, Immunology, 2003).

### BTA6 (88-96 Mb)

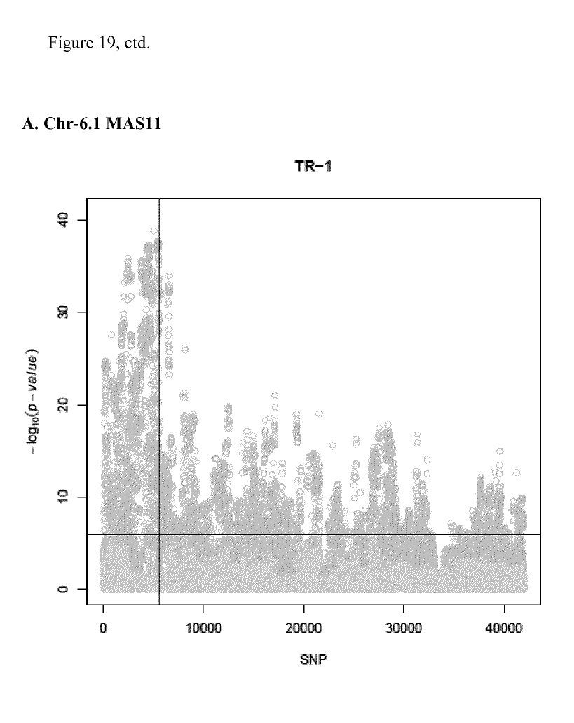

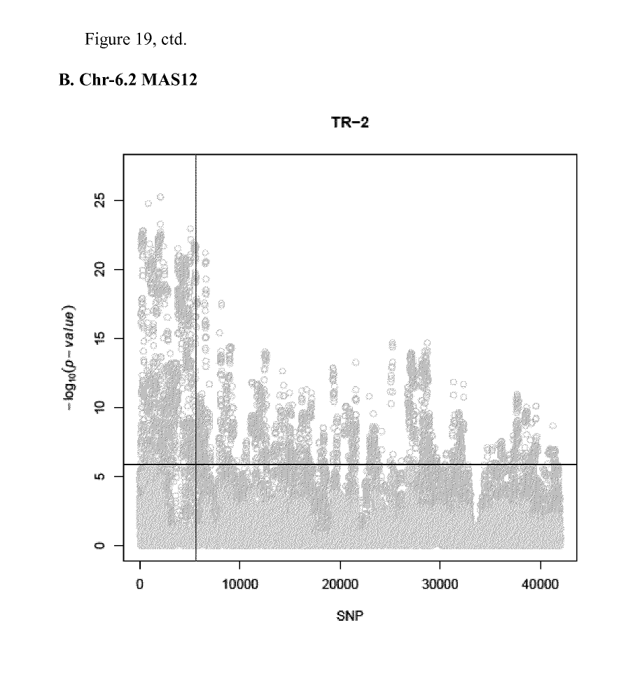

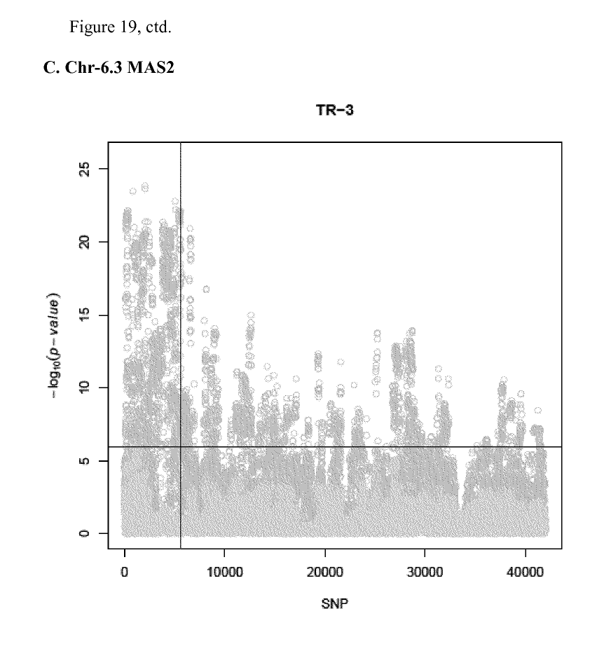

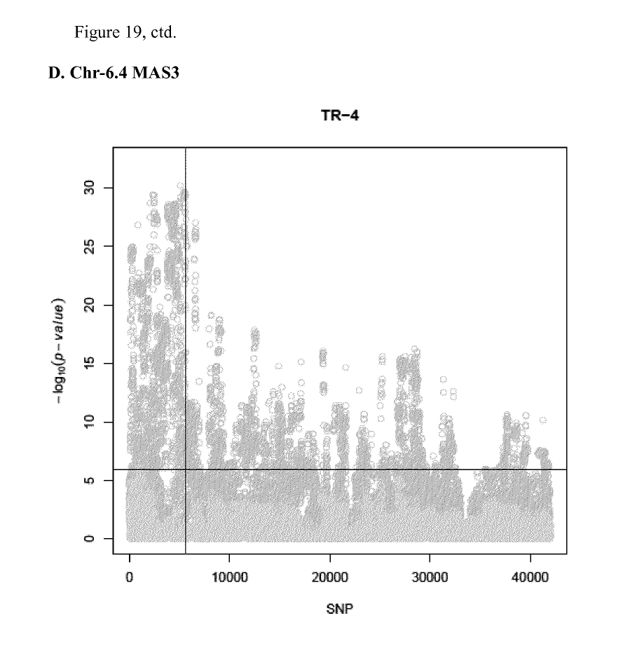

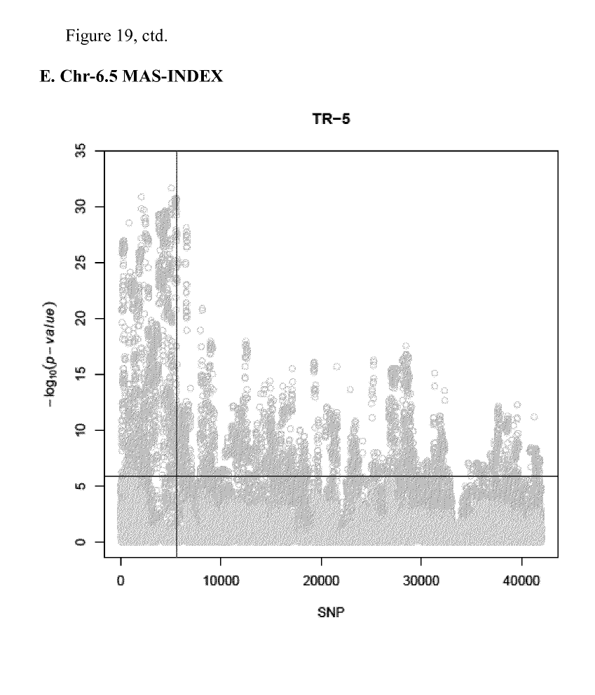

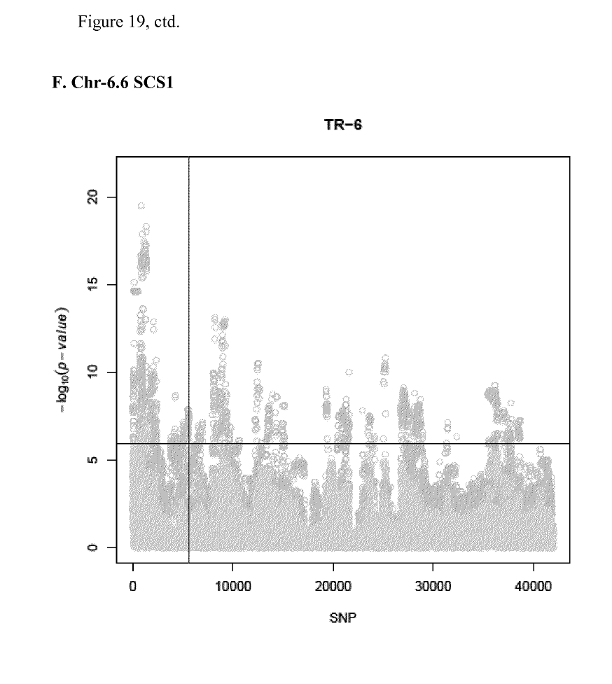

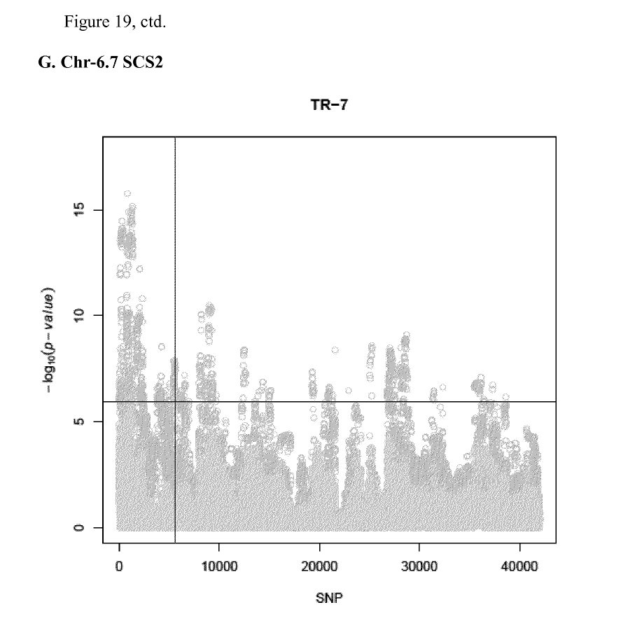

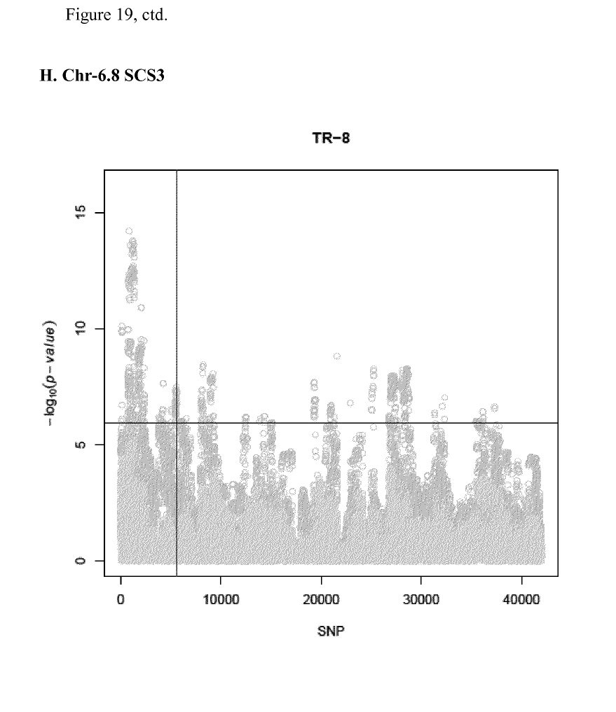

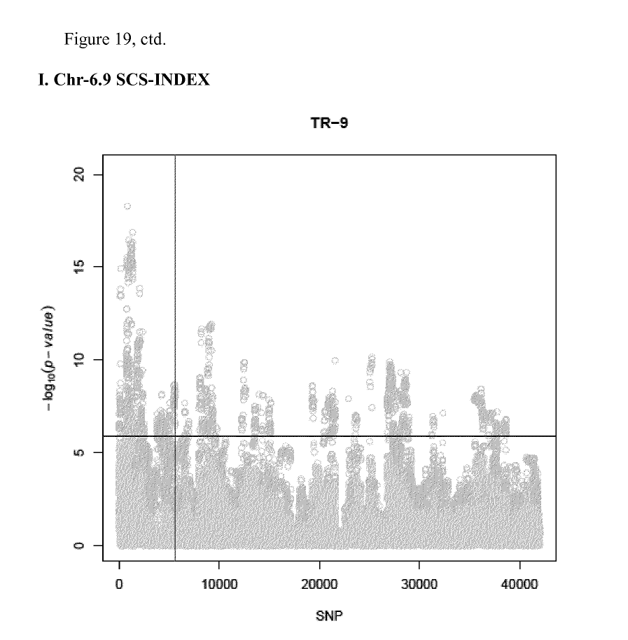

The most significant SNP for each of the nine mastitis related traits are presented in table 12 for the targeted region of BTA6. The targeted region on BTA6 was 8 Mb in length. The manhatton plot for this region is presented in the FIG. 19.

TABLE 12
The most significant SNP association for nine mastitis traits in the targeted region on BTA6.

| | | | | | | | | Allele |
|---|---|---|---|---|---|---|---|---|
| | | SNP | | | | | | increasing |
| | | position | | b- | | -log₁₀(p- | | mastitis |
| Trait | SNP name | (Bp) | MAF | value | SE | value) | Genotype | resistance |
| CM11 | Chr6_88977023 | 88977023 | 0.432 | −2.800 | 0.211 | 38.76 | C/T | C |
| CM12 | Chr6_88612186 | 88612186 | 0.403 | −2.772 | 0.262 | 25.27 | G/T | G |
| CM2 | Chr6_88610743 | 88610743 | 0.169 | −5.945 | 0.578 | 23.84 | T/A | T |
| CM3 | Chr6_88977023 | 88977023 | 0.432 | −2.447 | 0.210 | 30.21 | C/T | C |
| CM | Chr6_88977023 | 88977023 | 0.432 | −2.493 | 0.209 | 31.66 | C/T | C |
| SCS1 | Chr6_88326504 | 88326504 | 0.124 | −6.134 | 0.124 | 19.45 | G/A | G |
| SCS2 | Chr6_88326504 | 88326504 | 0.124 | −5.756 | 0.697 | 15.75 | G/A | G |
| SCS3 | Chr6_88326504 | 88326504 | 0.124 | −5.738 | 0.734 | 14.19 | G/A | G |
| SCS | Chr6_88326504 | 88326504 | 0.124 | −5.886 | 0.659 | 18.25 | G/A | G |

### Candidate Polymorphism for the BTA6 Targeted Region:

SNP, Chr6_{—}89059253, is a strong candidate polymorphism (alt 48%, gene ENSBTAG00000009070) for the targeted region of BTA6. This SNP showed very strong association with all the five clinical mastitis traits (CM11, CM12, CM2, CM3 and CM) (Table 13).

TABLE 13
The most associated polymorphism SNP from annotation were located at 89,059,253 on
BTA6. This SNP show high association with all the five clinical mastitis traits.

| | | | | | | Allele |
|---|---|---|---|---|---|---|
| | | | | | | increasing |
| | SNP position | | | | | mastitis |
| SNP-name | (BP) | trait | MAF | -log₁₀(p-value) | Genotype | resistance |
| Chr6_89059253 | 89059253 | CM11 | 0.483 | 37.40 | G/A | G |
| Chr6_89059253 | 89059253 | CM12 | 0.483 | 21.68 | G/A | G |
| Chr6_89059253 | 89059253 | CM2 | 0.483 | 22.08 | G/A | G |
| Chr6_89059253 | 89059253 | CM3 | 0.483 | 29.34 | G/A | G |
| Chr6_89059253 | 89059253 | CM | 0.483 | 30.62 | G/A | G |
| Chr6_89059253 | 89059253 | SCSI | 0.483 | 7.39 | G/A | G |
| Chr6_89059253 | 89059253 | SCS2 | 0.483 | 7.56 | G/A | G |
| Chr6_89059253 | 89059253 | SCS3 | 0.483 | 7.21 | G/A | G |
| Chr6_89059253 | 89059253 | SCS | 0.483 | 8.30 | G/A | G |

TABLE 14
BTA6: Genes associated with mastitis according to the present analysis. For
clinical mastitis the top SNPs are concentrated around 88.9 Mb, whereas the
major peak for SCS is centered on 88.4 MB. Here we find the following genes:

| | | Associated | Gene location |
|---|---|---|---|
| Ensembl Gene ID | Description | gene name | (UMD3.1) |
| ENSBTAG00000018531 | Immunoglobulin J chain | IGJ | 87,759,438-87,768,834 |
| ENSBTAG00000009310 | UTP3, small subunit (SSU) pro- | UTP3 | 87,798,136-87,799,560 |
| | cessome component, homolog | | |
| | (S. cerevisiae) | | |
| ENSBTAG00000016795 | RUN and FYVE domain containing 3 | RUFY3 | 87,819,398-87,910,688 |
| ENSBTAG00000008577 | G-rich sequence factor 1 | GRSF1 | 87,922,395-87,941,062 |
| ENSBTAG00000016290 | MOB kinase activator 1B | MOB1B | 87,976,520-88,030,195 |
| ENSBTAG00000012397 | Deoxycytidine kinase | DCK | 88,049,498-88,077,488 |
| ENSBTAG00000002348 | Electrogenic sodium bicarbonate | SLC4A4 | 88,182,303-88,541,046 |
| | cotransporter 1 | | |
| ENSBTAG00000013718 | Vitamin D-binding protein precursor | GC | 88,695,940-88,739,180 |
| ENSBTAG00000009070 | Neuropeptide FF receptor 2 | NPFFR2 | 89,052,210-89,059,348 |
| ENSBTAG00000006507 | ADAM metallopeptidase with | ADAMTS3 | 89,162,542-89,460,195 |
| | thrombospondin type 1 motif, 3 | | |

One associated gene is the IGJ gene, which encodes the immunoglobulin J polypeptide although it should be noted that the gene might be located too far away from the peak. This protein interacts with immunoglobulins IgM and IgA. IgM is the first antibody produced in the primary immune response to microbial infections whereas IgA is engaged in the defense against microorganisms in particular those invading the host through mucosal surfaces. Another associated gene is Deoxycytidine kinase (DCK gene), which catalyzes the rate-determining step in the deoxyribonucleoside salvage pathway. DCK is expressed in thymus and bone marrow, possibly indicating a role in lymphopoiesis. Mice lacking DCK enzyme activity revealed a combined immune deficiency phenotype, i.e. they produce very low levels of both T and B lymphocytes (Toy et al., PNAS, 2010). A relevant gene in this region is the GC gene, which belongs to the albumin family. The GC protein binds vitamin D and is involved in (inflammationprimed) activation of macrophages (Yamamoto and Naraparaju, Journal of Immunology, 1996; Kisker et al., Neoplasia, 2003). Another gene associated with mastitis in this region is the NPFFR2 gene (also known as GPR74), which encodes neuropeptide FF receptor 2. NPFFR2 show expression in several tissues including thymus, liver, spleen, brain, spinal cord and other. NPFF receptors have been implicated in hormonal modulation, regulation of food intake, thermoregulation and nociception through modulation of the opioid system (information from GeneCards). However, it is well documented that many neuropeptides participate in immune responses for example by acting as stimulators or inhibitors of macrophage activity (reviewed by Ganea and Delgado, Microbes and Infection, 2001). NPFFR2 also binds the prolactin-releasing-hormone, suggesting that NPFFR2 may play a role in prolactin secretion (Ma et al., European journal of neuroscience, 2009). Interestingly, in addition to regulating lactation, prolactin also acts as an important regulator of the immune system (Yu-lee, Recent Progress in Hormone Research, 2002).

### BTA13 (57-63 Mb)

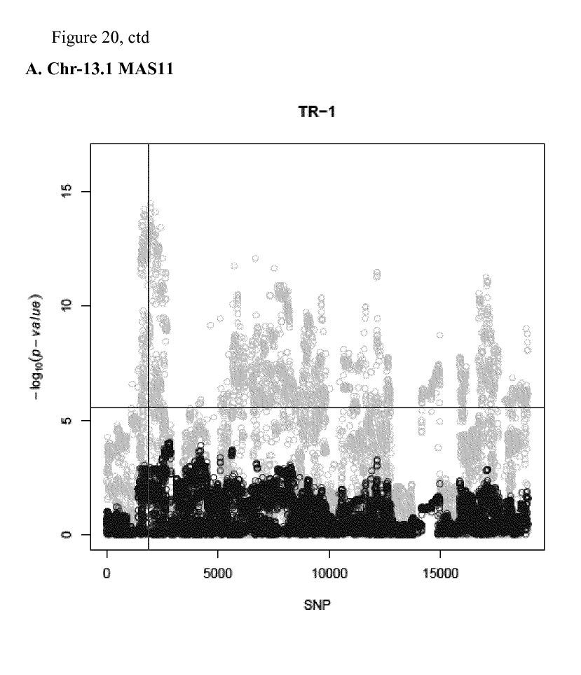

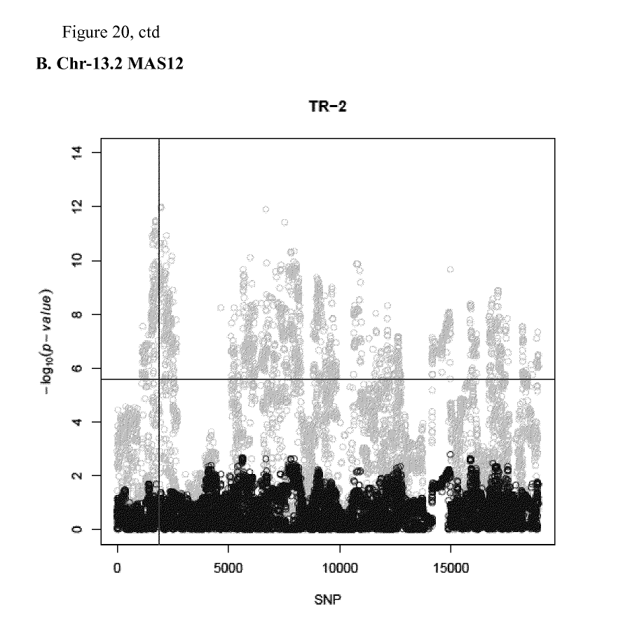

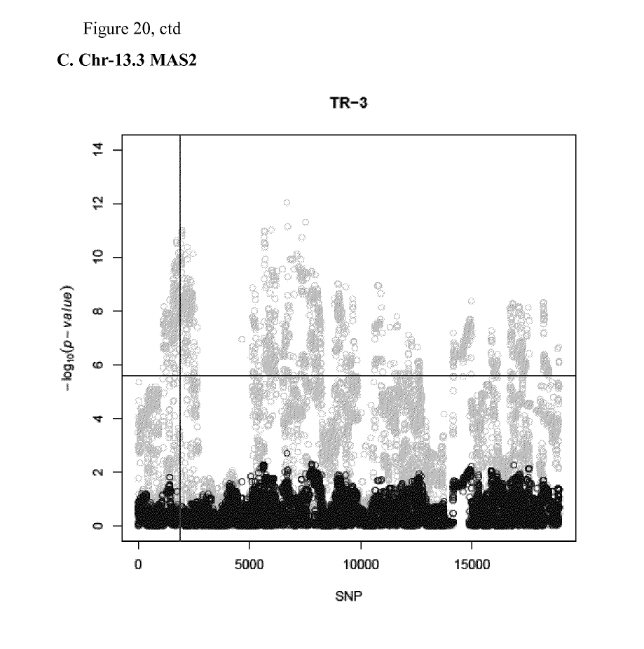

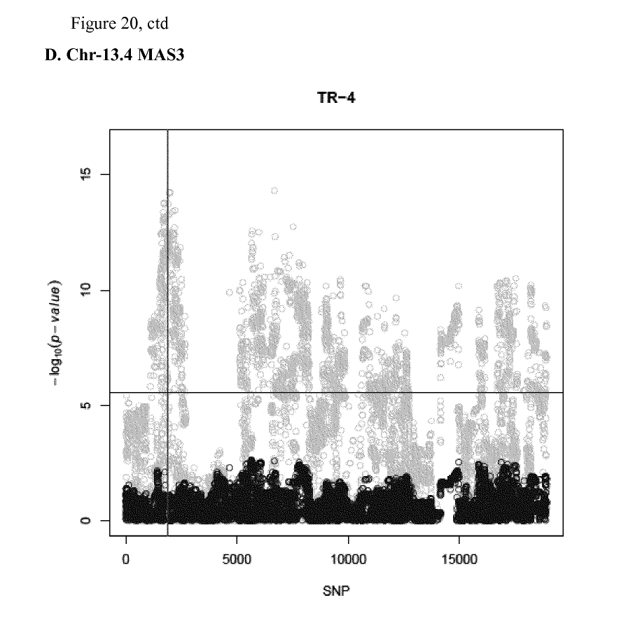

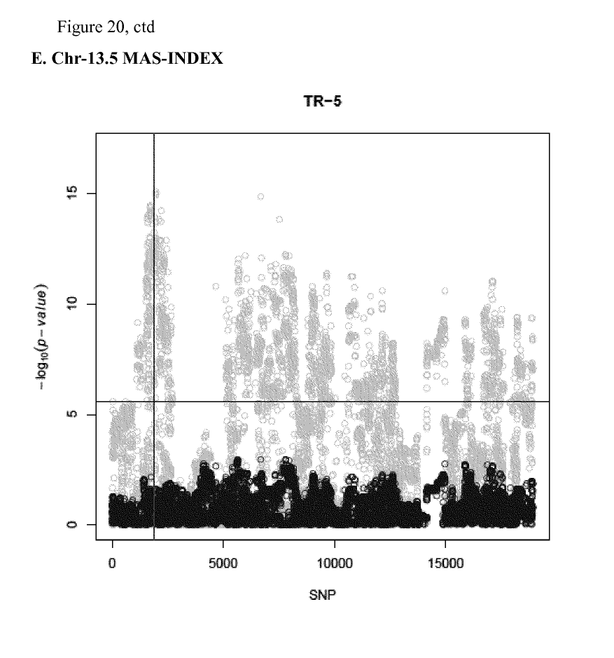

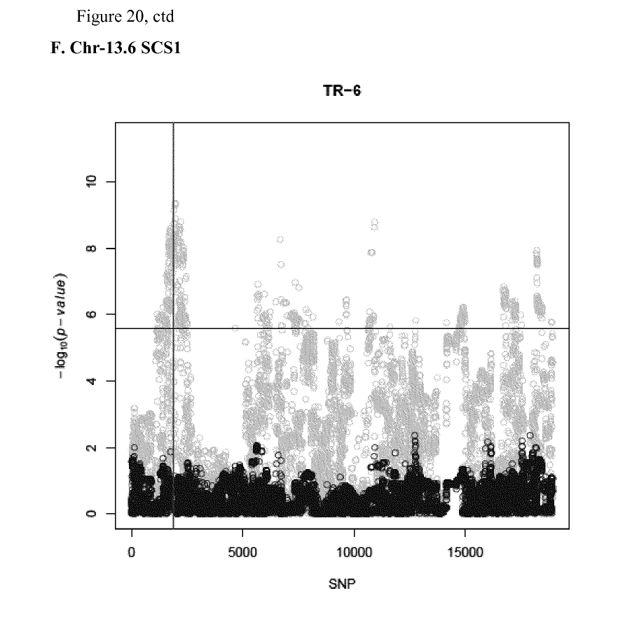

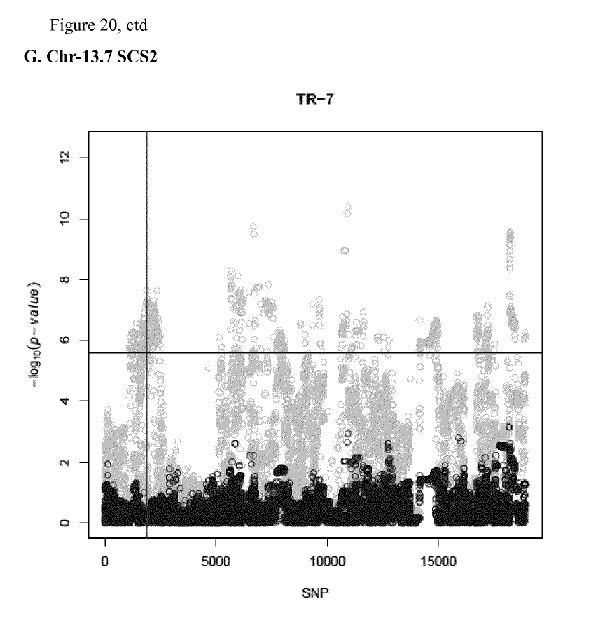

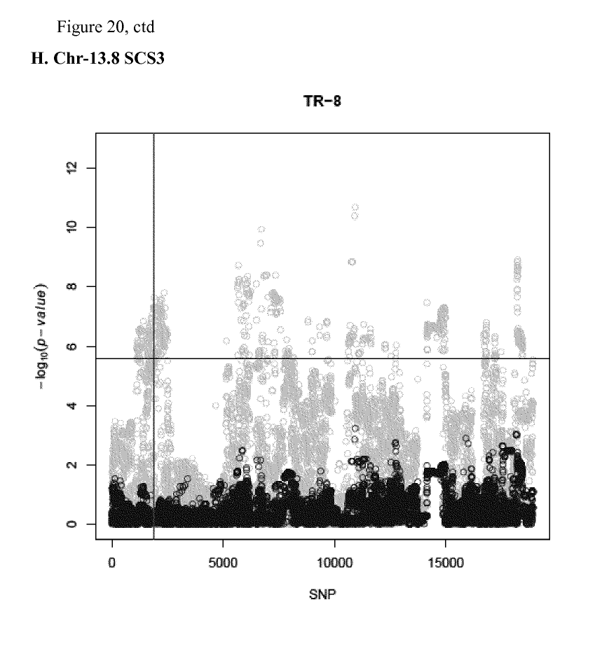

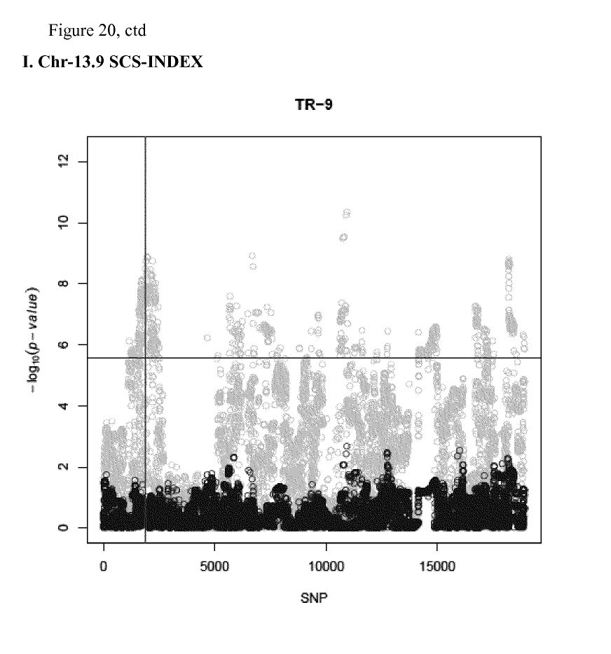

The most significant SNP for each of the nine mastitis related traits for the targeted region of BTA13 are presented in table 15. The targeted region was 6 Mb in length. The manhatton plot for this region is presented in the FIG. 20.

TABLE 15
The most significant SNP association for nine mastitis traits in the targeted region on BTA13

| | | | | | | | | Allele |
|---|---|---|---|---|---|---|---|---|
| | | | | | | | | increasing |
| | | Position | | | | -log₁₀(p- | | mastitis |
| Trait | Top-SNP | (Bp) | MAF | b-value | SE | value) | Genotype | resistance |
| CM11 | Chr13_57608336 | 57608336 | 0.072 | −8.127 | 1.029 | 14.46 | A/C | A |
| CM12 | Chr13_57608354 | 57608354 | 0.294 | −1.793 | 0.251 | 12.00 | A/G | A |
| CM2 | Chr13_59584651 | 59584651 | 0.234 | −6.433 | 0.899 | 12.02 | T/G | T |
| CM3 | Chr13_59584651 | 59584651 | 0.234 | −6.728 | 0.857 | 14.32 | T/G | T |
| CM | Chr13_57608628 | 57608628 | 0.305 | −1.908 | 0.236 | 15.07 | A/G | A |
| SCS1 | Chr13_57608354 | 57608354 | 0.294 | −1.619 | 0.259 | 9.35 | A/G | A |
| SCS2 | Chr13_60621602 | 60621602 | 0.014 | −29.835 | 4.511 | 10.39 | A/G | A |
| SCS3 | Chr13_60621602 | 60621602 | 0.014 | −31.429 | 4.678 | 10.69 | A/G | A |
| SCS | Chr13_60621602 | 60621602 | 0.014 | −28.314 | 4.290 | 10.34 | A/G | A |

### Candidate Polymorphism for BTA13 Targeted Region:

Two possible candidate polymorphisms based on functional annotation within the targeted region of BTA13 could be two consecutive SNPs located at 57579568 and 57579569 and both of them showed very high associations with mastitis traits.

TABLE 16
Association results for the two most associated polymorphism SNPs
from annotation with clinical mastitis on BTA13.

| | | | | | | Allele |
|---|---|---|---|---|---|---|
| | SNP | | | | | increasing |
| | position | | | -log₁₀(p- | Geno- | mastitis |
| SNP-name | (BP) | trait | MAF | value) | type | resistance |
| Chr13_57579568 | 57579568 | CM11 | 0.094 | 13.22 | G/T | T |
| Chr13_57579568 | 57579568 | CM12 | 0.094 | 9.19 | G/T | T |
| Chr13_57579568 | 57579568 | CM2 | 0.094 | 8.23 | G/T | T |
| Chr13_57579568 | 57579568 | CM3 | 0.094 | 12.11 | G/T | T |
| Chr13_57579568 | 57579568 | CM | 0.094 | 12.59 | G/T | T |
| Chr13_57579568 | 57579568 | SCS1 | 0.094 | 8.22 | G/T | T |
| Chr13_57579568 | 57579568 | SCS2 | 0.094 | 5.80 | G/T | T |
| Chr13_57579568 | 57579568 | SCS3 | 0.094 | 5.62 | G/T | T |
| Chr13_57579568 | 57579568 | SCS | 0.094 | 7.71 | G/T | T |
| Chr13_57579569 | 57579569 | CM11 | 0.063 | 13.22 | C/G | G |
| Chr13_57579569 | 57579569 | CM12 | 0.063 | 9.20 | C/G | G |
| Chr13_57579569 | 57579569 | CM2 | 0.063 | 8.23 | C/G | G |
| Chr13_57579569 | 57579569 | CM3 | 0.063 | 12.11 | C/G | G |
| Chr13_57579569 | 57579569 | CM | 0.063 | 12.60 | C/G | G |
| Chr13_57579569 | 57579569 | SCS1 | 0.063 | 8.22 | C/G | G |
| Chr13_57579569 | 57579569 | SCS2 | 0.063 | 5.80 | C/G | G |
| Chr13_57579569 | 57579569 | SCS3 | 0.063 | 5.62 | C/G | G |
| Chr13_57579569 | 57579569 | SCS | 0.063 | 7.71 | C/G | G |

TABLE 17
BTA13. Genes associated with mastitis according to the present analysis.

| Ensembl Gene ID | Location | Gene name | Short name | Comments |
|---|---|---|---|---|
| ENSBTAG00000020261 | 57056797-57091107 | Cadherin 26 | CAD26 | Cadherins are a family of adhesion mole- |
| | | | | cules that mediate Ca2+-dependent cell- |
| | | | | cell adhesion in all solid tissues and |
| | | | | modulate a wide variety of processes, |
| | | | | including cell polarization and migration. |
| ENSBTAG00000012109 | 57571799-57596875 | Endothelin 3 | EDN3 | Endothelins are proteins that constrict |
| | | | | blood vessels and raise blood pressure. |
| | | | | endothelium family member Edn3, acting |
| | | | | through the endothelin receptor EdnrA. |
| | | | | This might mediate transport of energy |
| | | | | and other small molecules to specific |
| | | | | tissues. |
| ENSBTAG00000018053 | 58537701-58585721 | Ras-related | RAB22A | The protein encoded by this gene is a |
| | | protein Rab-22A | | member of the RAB family of small |
| | | | | GTPases. The GTP-bound form of the |
| | | | | encoded protein has been shown to in- |
| | | | | teract with early-endosomal antigen 1, |
| | | | | and may be involved in the trafficking of |
| | | | | and interaction between endosomal |
| | | | | compartments. Small GTPases of the |
| | | | | RAB family, such as RAB22A, are in- |
| | | | | volved in the transport of macromole- |
| | | | | cules along endocytic and exocytic path- |
| | | | | ways. |
| | 59.1 Mb | novel, 3 tran- | | blastp hit to “predicted: z-DNA binding |
| | | scripts | | protein 1 (Bos taurus)” and “DNA- |
| | | | | dependent activator of IFN-regulatory |
| | | | | factor (Sus scrofa)”. Could be interesting |
| | | | | if involved in interferon regulation. |
| | 60.2 Mb | novel protein | | Domains Ig-like. Having Ig-like domains |
| | | coding | | could indicate involvement in recognition |
| | | | | of other molecules. |
| ENSBTAG00000018418 | 60487257-60492005 | Transmem- | TMEM74B | TMEM74 is a lysosome and autophago- |
| | | brane protein | | some protein that plays a role in autoph- |
| | | 74B | | agy, however as human TMEM74 is lo- |
| | | | | cated on Hsa8 it is not the homologue of |
| | | | | this TMEM74B gene. |
| ENSBTAG00000013330 | 61123467-61142447 | TBC1 do- | TBC1D20 | Sklan et al. (2007) showed that reduction |
| | | main family | | of TBC1D20 expression by siRNA se- |
| | | member 20 | | verely impaired Hepatitis C Virus replica- |
| | | | | tion and inhibited new infection. Howev- |
| | | | | er, as this is a virus it might be a different |
| | | | | pathway and not relevant for mastitis. |
| ENSBTAG00000048288 | 61314568-61316738 | Defensin, | DEFB129 | The beta defensins are antimicrobial pep- |
| | | beta 129 | | tides implicated in the resistance of epi- |
| | | | | thelial surfaces to microbial colonization. |
| ENSBTAG00000003364 | 61523659-61533444 | Beta- | DEFB119 | |
| | | defensin 119 | | |
| ENSBTAG00000048009 | 61501526-61501651 | defensin, | DEFB117 | |
| | | beta 117 | | |
| ENSBTAG00000027384 | 61562053-61566096 | beta-defensin | DEFB122a | |
| | | 122a | | |
| ENSBTAG00000027383 | 61572838-61577455 | beta-defensin | DEFB122 | |
| | | 122 | | |
| ENSBTAG00000020555 | 61584391-61595672 | beta-defensin | DEFB123 | |
| | | 123 | | |
| ENSBTAG00000031254 | 61612683-61615456 | beta-defensin | DEFB124 | |
| | | 124 | | |
| ENSBTAG00000016169 | 61726125-61727283 | DNA-binding | ID1 | During B-cell differentiation, Id inhibitory |
| | | protein inhibi- | | proteins, particularly ID1 and ID2, are |
| | | tor ID-1 | | expressed at high levels in pro-B cells |
| | | | | (Sun et al., 1991; Wilson et al., 1991) and |
| | | | | are downregulated as cells differentiate |
| | | | | into pre-B and mature B cells, presum- |
| | | | | ably for the purpose of releasing the bHLH |
| | | | | proteins (e.g., E2A; 147141) that are im- |
| | | | | portant for differentiation. |
| | 61.9 Mb | Uncharacter- | | Blast shows similarity to “interferon regu- |
| | | ized protein | | latory factor 4”, which is a transcription |
| | | | | factor essential for the development of T |
| | | | | helper-2 (Th2) cells, IL17-producing Th17 |
| | | | | cells, and IL9-producing Th9 cells (Staudt |
| | | | | et al., 2010). |
| ENSBTAG00000016348 | 62030345-62054881 | XK, Kell | XKR7 | Blood groups are interesting as they of- |
| | | blood group | | ten presents a defense against macro- |
| | | complex | | molecules. The exact function of the Kell |
| | | subunit- | | blood groups has not been deduced. |
| | | related fami- | | |
| | | ly, member 7 | | |
| ENSBTAG00000019200 | 62850752-62869092 | BPI fold con- | BPIFB2 | BPIL1 shares significant similarity with |
| | | taining family | | members of the lipid transfer |
| | | B, member 2 | | (LT)/lipopolysaccharide (LPS)-binding |
| | | | | protein (LBP) family. All LT/LBP proteins |
| | | | | are capable of binding phospholipids and |
| | | | | LPS. Some are involved in lipid transfer |
| | | | | and metabolism (e.g., CETP), and others |
| | | | | are involved in host response to gram- |
| | | | | negative bacterial infection (e.g., BPI) |
| | | | | (summary by Mulero et al., 2002). |
| ENSBTAG00000010112 | 62877511-62892488 | BPI fold con- | BPIFB6 | BPI = bactericidal/permeability increasing |
| | | taining family | | |
| | | B, member 6 | | |
| ENSBTAG00000038687 | 62901440-62918251 | BPI fold con- | BPIFB3 | |
| | | taining family | | |
| | | B, member 3 | | |
| ENSBTAG00000038412 | 62927643-62950669 | BPI fold con- | BPIFB4 | |
| | | taining family | | |
| | | B, member 4 | | |
| | 63.0 Mb | Uncharacter- | | Blast shows similarity to “SPLUNC6” and |
| | | ized protein | | “+I89”. This seems related to BPI. |

### BTA16 (48-55 Mb)

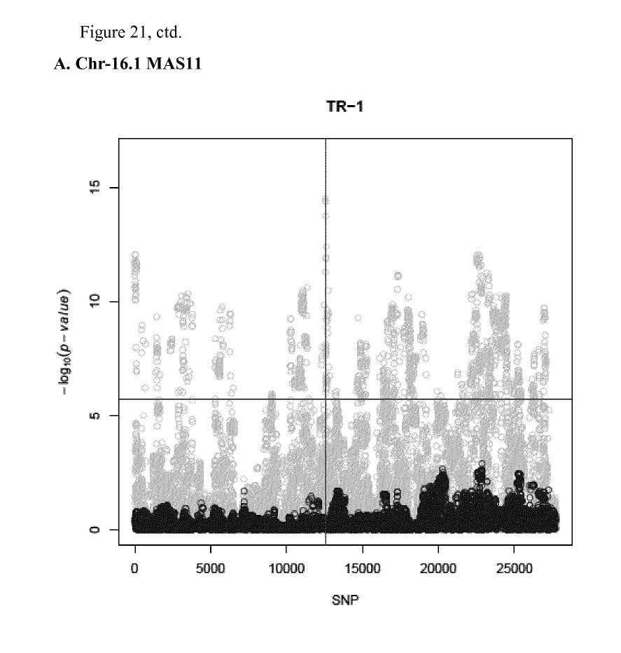

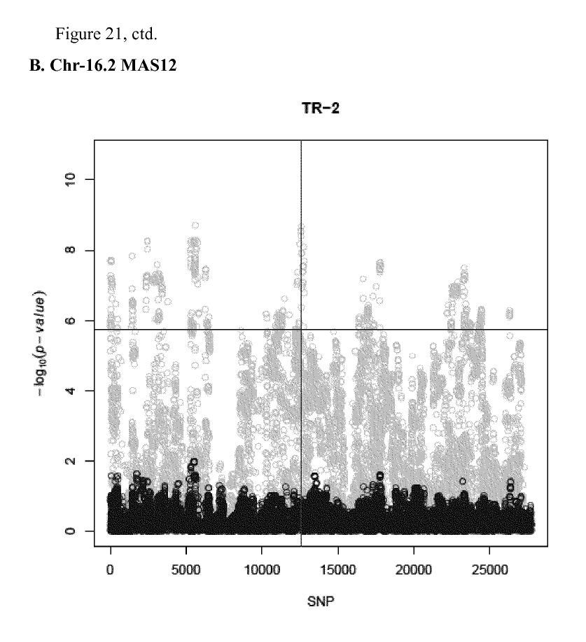

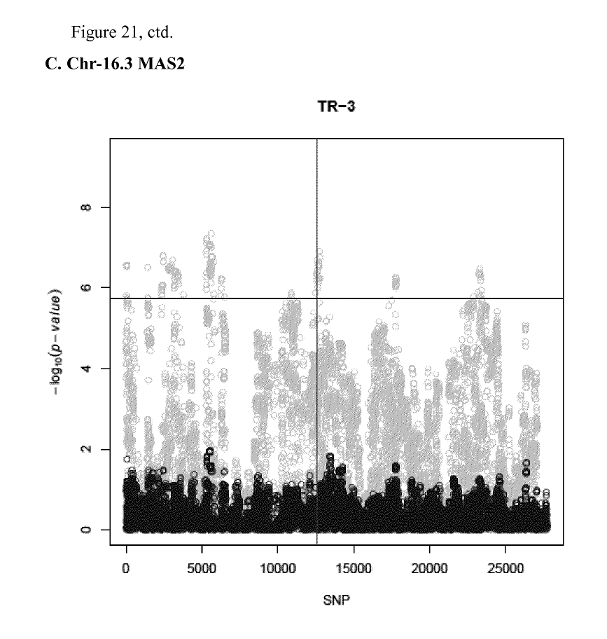

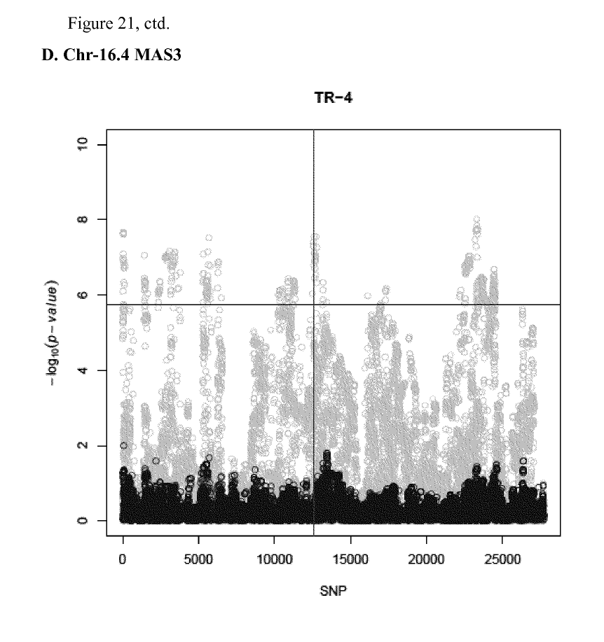

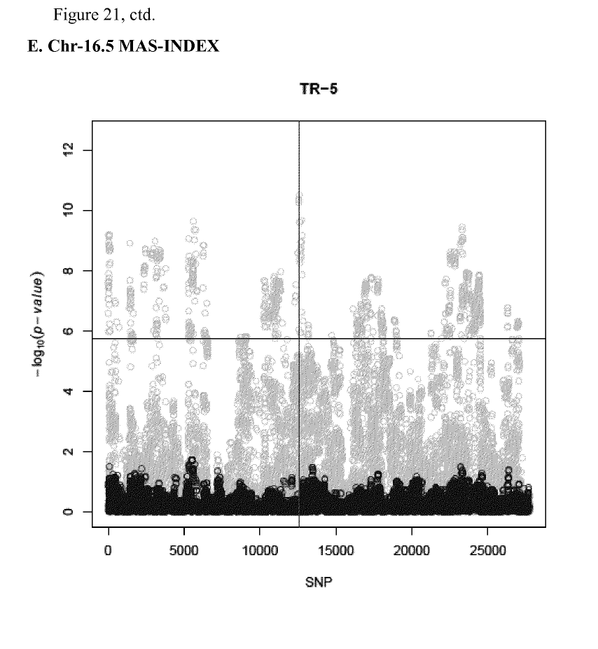

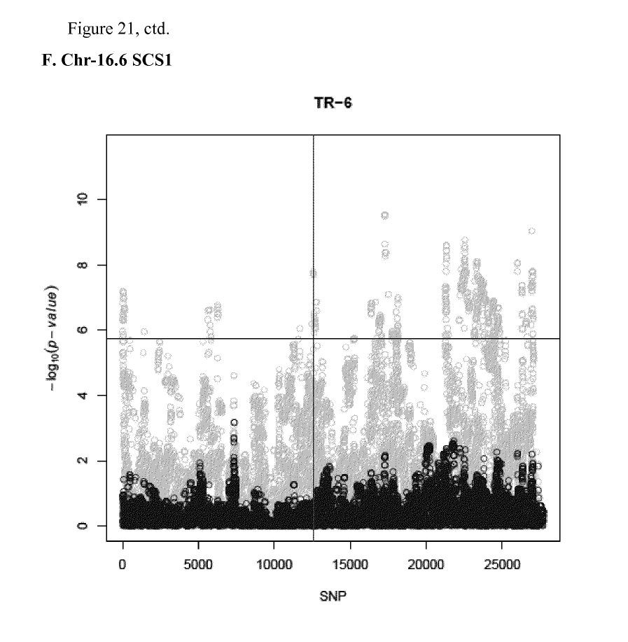

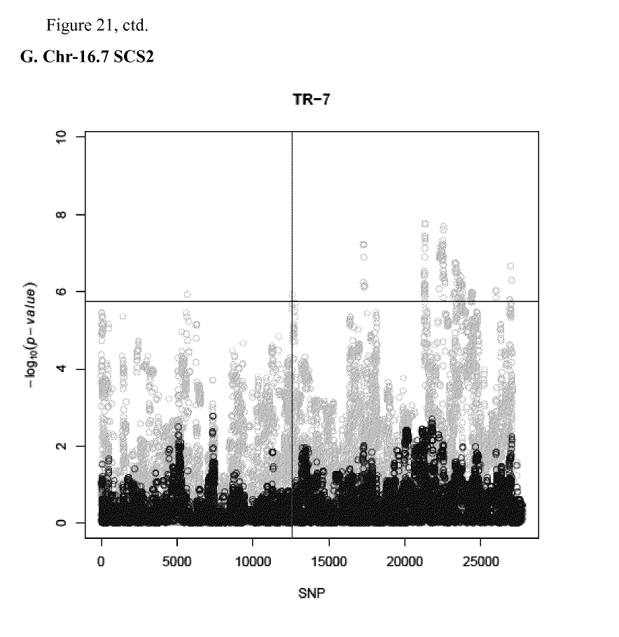

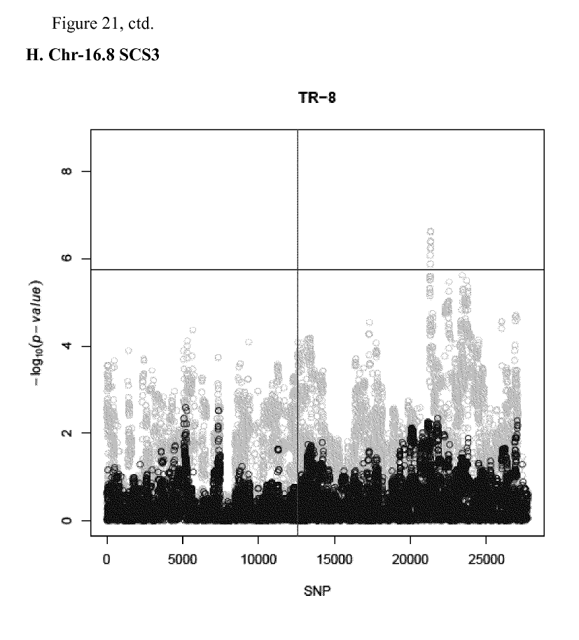

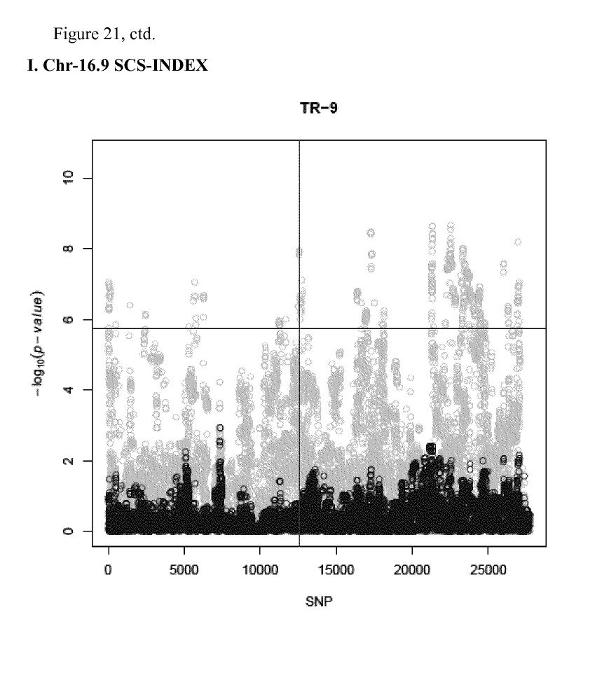

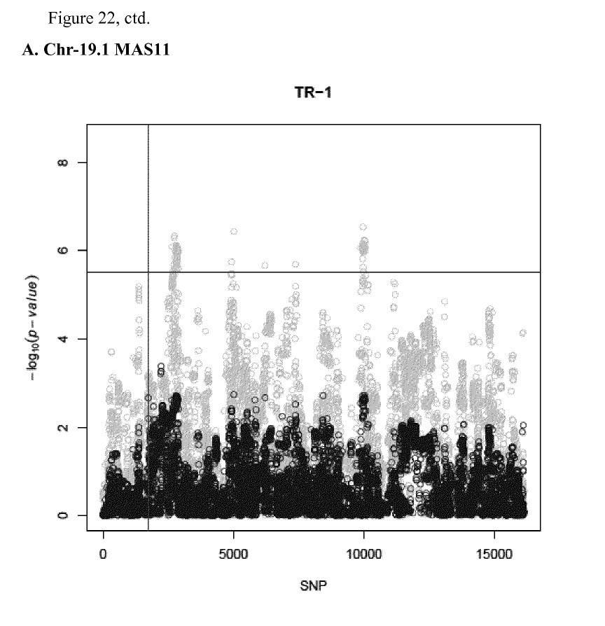

The most significant SNP for each of the nine mastitis related traits for the targeted region of BTA16 are presented in table 18. The targeted region on BTA16 was 7 Mb. The manhatton plot for this region is presented in the FIG. 21.

TABLE 18
The most significant SNP association for nine mastitis traits in the targeted region on BTA16

| | | | | | | | | Allele |
|---|---|---|---|---|---|---|---|---|
| | | SNP | | | | | | increasing |
| | | position | | | | -log₁₀(p- | | mastitis |
| Trait | SNP name | (Bp) | MAF | b-value | SE | value) | Genotype | resistance |
| CM11 | Chr16_50529178 | 50529178 | 0.019 | 28.704 | 3.628 | 14.51 | G/A | A |
| CM12 | Chr16_49054912 | 49054912 | 0.282 | 1.504 | 0.250 | 8.72 | C/T | T |
| CM2 | Chr16_49054912 | 49054912 | 0.282 | 1.416 | 0.259 | 7.34 | C/T | T |
| CM3 | Chr16_54246279 | 54246279 | 0.241 | 1.308 | 0.228 | 8.01 | C/A | A |
| CM | Chr16_50532600 | 50532600 | 0.306 | 1.663 | 0.250 | 10.49 | C/A | A |
| SCS1 | Chr16_52097973 | 52097973 | 0.052 | 11.676 | 1.849 | 9.53 | C/A | A |
| SCS2 | Chr16_53806663 | 53806663 | 0.449 | 1.317 | 0.233 | 7.78 | C/G | G |
| SCS3 | Chr16_53806663 | 53806663 | 0.449 | 1.260 | 0.234 | 6.61 | C/G | G |
| SCS | Chr16_53998150 | 53998150 | 0.169 | 6.124 | 1.022 | 8.66 | C/T | T |

### Candidate Polymorphism for BTA16 Targeted Region

The candidate SNPs for the targeted region on BTA16 which showed strong association across several mastitis related traits are presented in Table 19. The SNP at 50,529,178 showed the strong association followed by two more SNPs (50,564,280 and 50,573,032) across several traits. Besides these three candidates, there is a non-synonymous polymorphism at 50,529,395 (alt 8%) located in the gene ENSBTAG00000020014. Downstream there are candidates in ENSBTAG00000004738 at 50,546,994 (alt 45%) (non-synonymous), and a splice-site polymorphism at 50,547,815 (alt 78%).

TABLE 19
Association results for the strongest polymorphisms from annotation
with clinical mastitis traits on BTA16.

| | | | | | | Allele |
|---|---|---|---|---|---|---|
| | SNP | | | | | increasing |
| | position | | | -log₁₀(p- | Geno- | mastitis |
| SNP name | (Bp) | trait | MAF | value) | type | resistance |
| Chr16_50529178 | 50529178 | CM11 | 0.019 | 14.51 | G/A | A |
| Chr16_50529178 | 50529178 | CM12 | 0.019 | 8.39 | | |
| Chr16_50529178 | 50529178 | CM2 | 0.019 | 6.21 | G/A | A |
| Chr16_50529178 | 50529178 | CM3 | 0.019 | 7.30 | G/A | A |
| Chr16_50529178 | 50529178 | CM | 0.019 | 10.25 | G/A | A |
| Chr16_50529178 | 50529178 | SCS1 | 0.019 | 7.79 | G/A | A |
| Chr16_50529178 | 50529178 | SCS2 | 0.019 | 5.65 | G/A | A |
| Chr16_50529178 | 50529178 | SCS3 | 0.019 | 3.74 | G/A | A |
| Chr16_50529178 | 50529178 | SCS | 0.019 | 7.91 | G/A | A |
| Chr16_50564280 | 50564280 | CM11 | 0.248 | 9.32 | C/T | T |
| Chr16_50564280 | 50564280 | CM12 | 0.248 | 7.56 | C/T | T |
| Chr16_50564280 | 50564280 | CM2 | 0.248 | 6.60 | C/T | T |
| Chr16_50564280 | 50564280 | CM3 | 0.248 | 7.07 | C/T | T |
| Chr16_50564280 | 50564280 | CM | 0.248 | 8.96 | C/T | T |
| Chr16_50564280 | 50564280 | SCS1 | 0.248 | 6.30 | C/T | T |
| Chr16_50564280 | 50564280 | SCS2 | 0.248 | 5.11 | C/T | T |
| Chr16_50564280 | 50564280 | SCS3 | 0.248 | 3.80 | C/T | T |
| Chr16_50564280 | 50564280 | SCS | 0.248 | 6.55 | C/T | T |
| Chr16_50573032 | 50573032 | CM11 | 0.254 | 10.49 | G/T | T |
| Chr16_50573032 | 50573032 | CM12 | 0.254 | 8.08 | G/T | T |
| Chr16_50573032 | 50573032 | CM2 | 0.254 | 6.92 | G/T | T |
| Chr16_50573032 | 50573032 | CM3 | 0.254 | 7.55 | G/T | T |
| Chr16_50573032 | 50573032 | CM | 0.254 | 9.68 | G/T | T |
| Chr16_50573032 | 50573032 | SCS1 | 0.254 | 6.85 | G/T | T |
| Chr16_50573032 | 50573032 | SCS2 | 0.254 | 5.50 | G/T | T |
| Chr16_50573032 | 50573032 | SCS3 | 0.254 | 3.94 | G/T | T |
| Chr16_50573032 | 50573032 | SCS | 0.254 | 7.12 | G/T | T |

TABLE 20
BTA16: Genes associated with mastitis according to the present analysis.

| Ensembl Gene ID | Location | Gene name | Short name | Comments |
|---|---|---|---|---|
| ENSBTAG00000024663 | 49272707-49285532 | Ladinin 1 | LAD1 | Ladinin is an anchoring filament protein |
| | | | | of basement membrane at the dermal- |
| | | | | epidermal junction. Human ladinin is an |
| | | | | autoantigen associated with linear IgA |
| | | | | disease |
| ENSBTAG00000016057 | 49332770-49353517 | Cysteine and | CSRP1 | CSRP1 is a member of the CSRP family |
| | | glycine-rich | | of genes encoding a group of LIM do- |
| | | protein 1 | | main proteins, which may be involved in |
| | | | | regulatory processes important for devel- |
| | | | | opment and cellular differentiation. The |
| | | | | LIM/double zinc-finger motif found in |
| | | | | CRP1 is found in a group of proteins with |
| | | | | critical functions in gene regulation, cell |
| | | | | growth, and somatic differentiation |
| ENSBTAG00000010732 | 52260743-52263073 | matrix metal- | MMP23B | The MMPs belong to a larger family of |
| | | loproteinase- | | proteases known as the metzincin super- |
| | | 23 precursor | | family. Collectively they are capable of |
| | | | | degrading all kinds of extracellular matrix |
| | | | | proteins, but also can process a number |
| | | | | of bioactive molecules. They are known |
| | | | | to be involved in the cleavage of cell sur- |
| | | | | face receptors, the release of apoptotic |
| | | | | ligands (such as the FAS ligand), and |
| | | | | chemokine/cytokine in/activation. MMPs |
| | | | | are also thought to play a major role on |
| | | | | cell behaviors such as cell proliferation, |
| | | | | migration (adhesion/dispersion), differen- |
| | | | | tiation, angiogenesis, apoptosis and host |
| | | | | defense. In humans duplicated (MMP |
| | | | | and CDC2) in a tail to tail fashion. Appar- |
| | | | | ently not in cattle. |
| ENSBTAG00000015635 | 52484468-52487309 | tumor necro- | TNFRSF4 | Although several membrane receptors |
| | | sis factor | | impact NF-kappaB activation, signaling |
| | | receptor su- | | from OX40 (CD134, TNFRSF4), a mem- |
| | | perfamily | | ber of the tumor necrosis factor receptor |
| | | member 4 | | (TNFR) superfamily, has proven to be |
| | | | | important for T cell immunity and a strong |
| | | | | contributor to NF-kappaB activity. |
| ENSBTAG00000015632 | 52492065-52494746 | tumor necro- | TNFSRF18 | |
| | | sis factor | | |
| | | receptor su- | | |
| | | perfamily, | | |
| | | member 18 | | |
| ENSBTAG00000014707 | 52714627-52715665 | Ubiquitin-like | ISG15 | ISG15 is secreted from monocytes in |
| | | protein | | response to type I IFNs and causes natu- |
| | | ISG15 | | ral killer (NK)-cell proliferation and an |
| | | | | augmentation of non-MCH (major histo- |
| | | | | compatibility complex)-restricted cytotox- |
| | | | | icity. ISG15 contains a unique subtype of |
| | | | | IFN-stimulated response element (ISRE) |
| | | | | that allows the binding of both PU.1 and |
| | | | | IRFs and the synergistic activation of the |
| | | | | element by the heterocomplex. |
| | 52.7 | Uncharacter- | | Blast showed weak similarity to “igA FC |
| | | ized protein | | receptor (Streptococcus) surface pro- |
| | | C1orf170 | | teinPspC (Streptococcus)”. If there is a |
| | | homolog | | significant resemblance to the presenting |
| | | | | molecule in Streptococcus, there might |
| | | | | be a relation to the immune defense |
| | | | | recognition of streptococcus or other bac- |
| | | | | teria. |
| ENSBTAG00000014537 | 52748704-52755937 | pleckstrin | PLEKHN1 | Some of the PLEKH (not necessarily |
| | | homology | | family N member 1) proteins are involved |
| | | domain con- | | in the signaling pathway of NFKB1 which |
| | | taining, fami- | | have been detected in cell types express- |
| | | ly N member | | ing cytokines, chemokines and acute |
| | | 1 | | phase proteins. The involvement in the |
| | | | | acute response can therefore not be |
| | | | | ruled out. |
| | 53.1 | Uncharacter- | | BLAST shows similarity to PLEKHM2. |
| | | ized protein | | Some of the PLEKH (not necessarily |
| | | | | family M member 2) proteins are involved |
| | | | | in the signaling pathway of NFKB1 which |
| | | | | have been detected in cell types express- |
| | | | | ing cytokines, chemokines and acute |
| | | | | phase proteins. The involvement in the |
| | | | | acute response can therefore not be |
| | | | | ruled out. |
| ENSBTAG00000037523 | 52467804-52468793 | UDP- | B3GALT6 | There is no info onB3GALT6 but other |
| | | Gal: betaGal | | members of the family are interesting. |
| | | beta 1,3- | | B3GALT5: Sequence analysis revealed |
| | | galactosyl- | | that the predicted 310-amino acid protein |
| | | transferase | | is a type II membrane protein, like other |
| | | polypeptide 6 | | glycosyltransferases. It has been demon- |
| | | | | strated that the beta-3-GalT5 enzyme is |
| | | | | the most probable candidate for the syn- |
| | | | | thesis of type 1 Lewis antigens in gastro- |
| | | | | intestinal and pancreatic cancers. |
| | | | | B3GALT3 encodes beta-1,3-N- |
| | | | | acetylgalactosaminyltransferase (EC |
| | | | | 2.4.1.79), an enzyme that catalyzes the |
| | | | | addition of GalNAc onto globotriaosylcer- |
| | | | | amide (GB3), the P(k) blood group anti- |
| | | | | gen, to form GB4, the P blood group an- |
| | | | | tigen. P(k) is synthesized by alpha-1,4- |
| | | | | galactosyltransferase (A4GALT). |

### BTA19 (55-58 Mb)

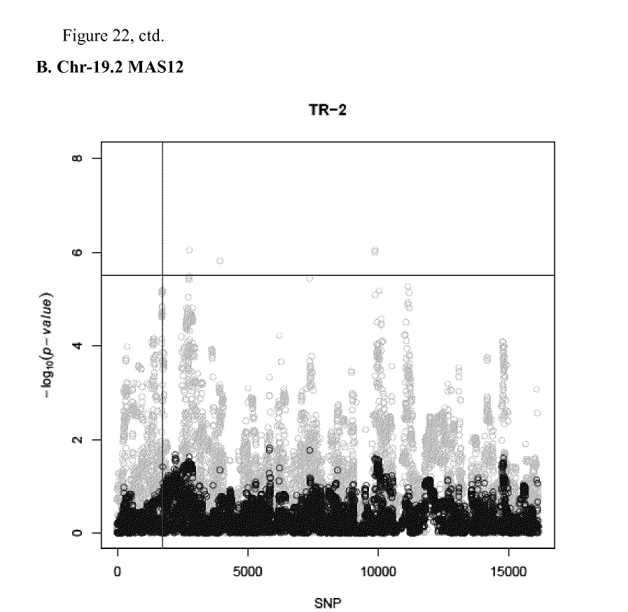

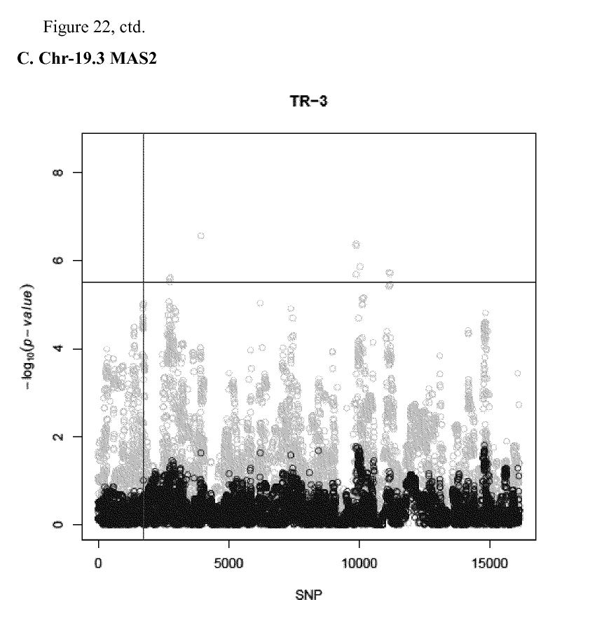

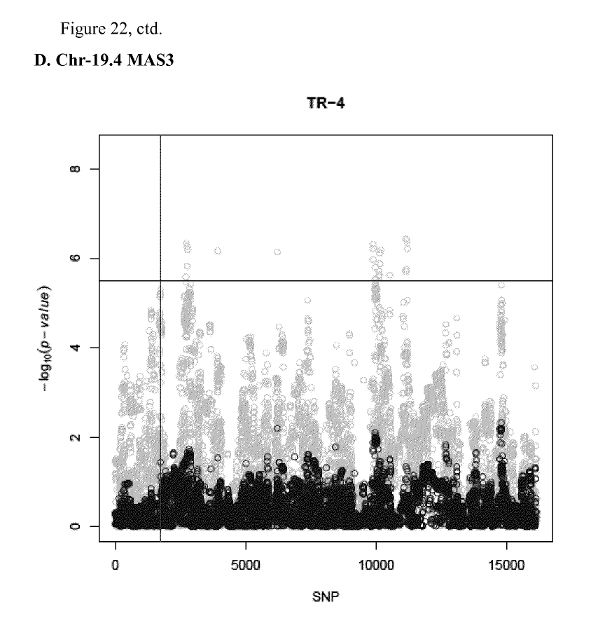

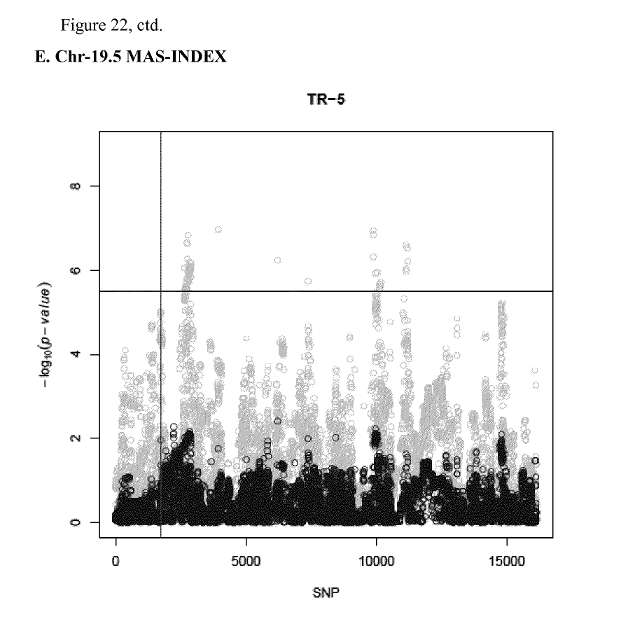

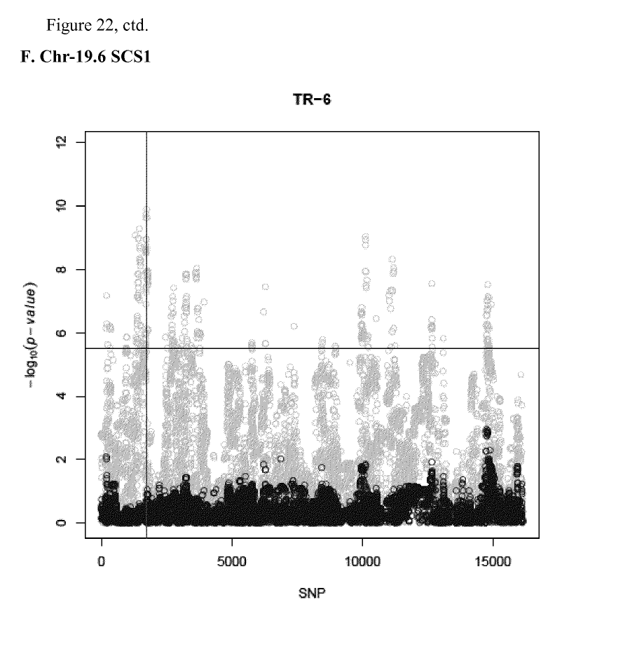

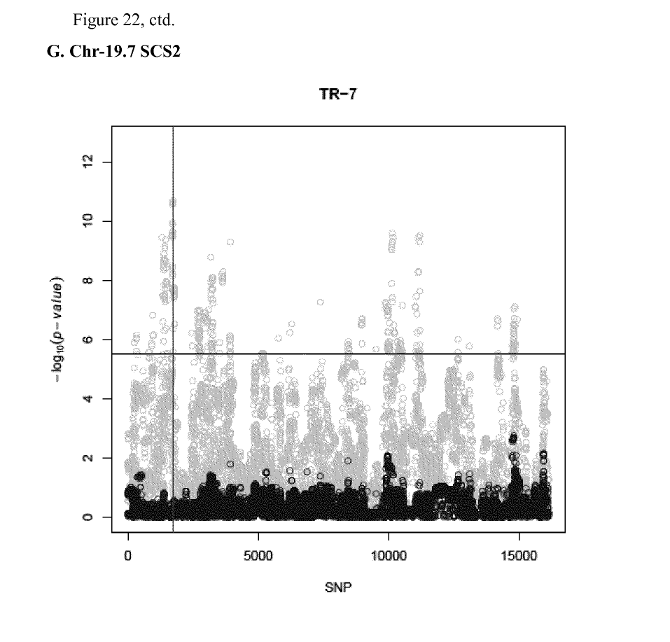

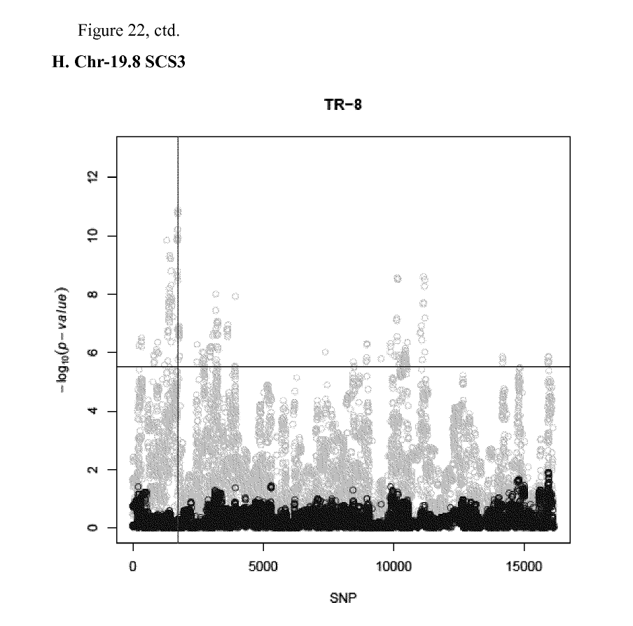

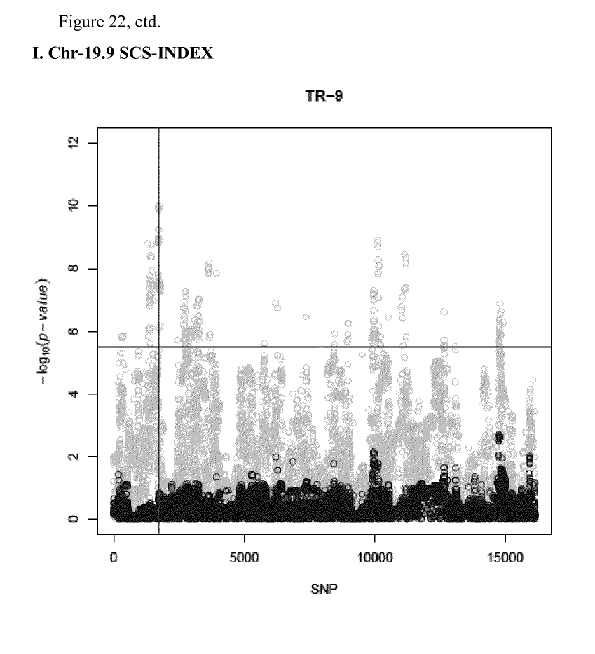

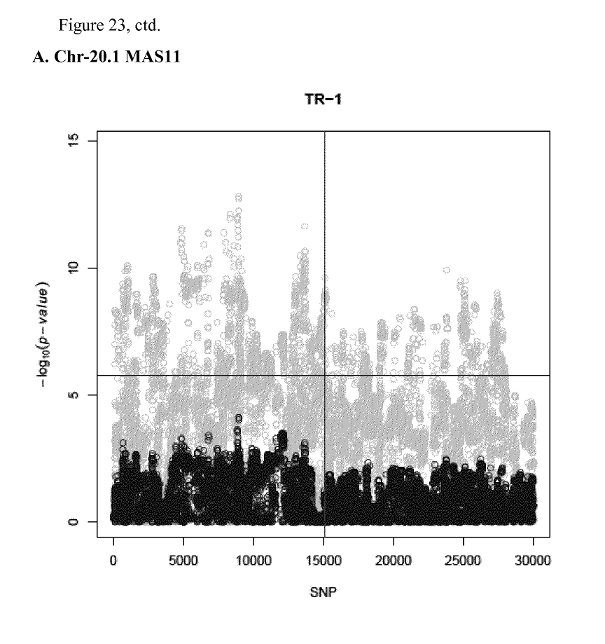

The most significant SNP for each of the nine mastitis related traits for the targeted region of BTA19 are presented in table 21. The targeted region on BTA19 was 3 Mb. The manhatton plot for this region is presented in the FIG. 22.

TABLE 21
The most significant SNP association for nine mastitis traits in the targeted region on BTA19

| | | | | | | | | Allele |
|---|---|---|---|---|---|---|---|---|
| | | SNP | | | | | | increasing |
| | | position | | b- | | -log₁₀(p- | | mastitis |
| Trait | SNP name | (Bp) | MAF | value | SE | value) | Genotype | resistance |
| CM11 | Chr19_57164311 | 57164311 | 0.293 | −2.377 | 0.463 | 6.53 | G/A | G |
| CM12 | Chr19_55461224 | 55461224 | 0.418 | 8.581 | 1.74 | 6.05 | A/C | C |
| CM2 | BovineHD1900015719 | 55615219 | 0.245 | 1.295 | 0.251 | 6.57 | G/A | A |
| CM3 | Chr19_57418222 | 57418222 | 0.350 | −1.154 | 0.227 | 6.43 | A/G | A |
| CM | BovineHD1900015719 | 55615219 | 0.245 | 1.246 | 0.234 | 6.95 | G/A | A |
| SCS1 | Chr19_55296191 | 55296191 | 0.380 | −1.632 | 0.253 | 9.90 | T/G | T |
| SCS2 | Chr19_55296191 | 55296191 | 0.380 | −1.786 | 0.266 | 10.71 | T/G | T |
| SCS3 | Chr19_55296191 | 55296191 | 0.380 | −1.883 | 0.278 | 10.90 | T/G | T |
| SCS | Chr19_55296191 | 55296191 | 0.380 | −1.632 | 0.251 | 10.03 | T/G | T |

Polymorphism Associated with Mastitis Resistance in the Targeted BTA19:

Downstream ENSBTAG00000013677 starts around 55,324,679 Bp (alt 72%). There are splice-site variants at 55,331,001 (alt 21%) and 55,338,316 (alt 64%). ENSBTAG00000044443 starts around 55,414,846 (not included in the association analyses). There is a variant in a non-coding gene at 55,419,720 (alt 29%). Upstream ENSBTAG00000002633 starts around 55,158,662 without any interesting polymorphisms. None of the above SNP selected from the functional annotation showed strong association signal across mastitis traits.

TABLE 22
BTA19: Genes associated with mastitis according to the present analysis.

| | | | Preliminary |
|---|---|---|---|
| Ensemble Id | Gene location (UMD3.1) | Common Gene Name | arguments |
| ENSBTAG00000013677 | 55,328,989-55,376,388 | SEC14-like protein 1 | Secretory protein ex- |
| | | | pressed a.o. in saliva, |
| | | | breast tissue. Potential |
| | | | SNP with effect upon |
| | | | splice site variants |
| ENSBTAG00000005104 | 55,528,770-55,590,603 | N-acetylglucosaminyltranferase VB | functions in |
| | | Ikke et standardnavn, det rigtige navn er | the synthesis |
| | | formodentligt: ALPHA-1,6-MANNOSYL- | of complex |
| | | GLYCOPROTEIN BETA-1,6-N- | cell surface |
| | | ACETYLGLUCOSAMINYLTRANSFERASE, | N-glycans |
| | | ISOZYME B; MGAT5B (comparative data) | |
| ENSBTAG00000044443 | 55,419,632-55,419,819 | Small Cajal body specific RNA 16 | Little info |

### BTA20 (32-40 Mb)

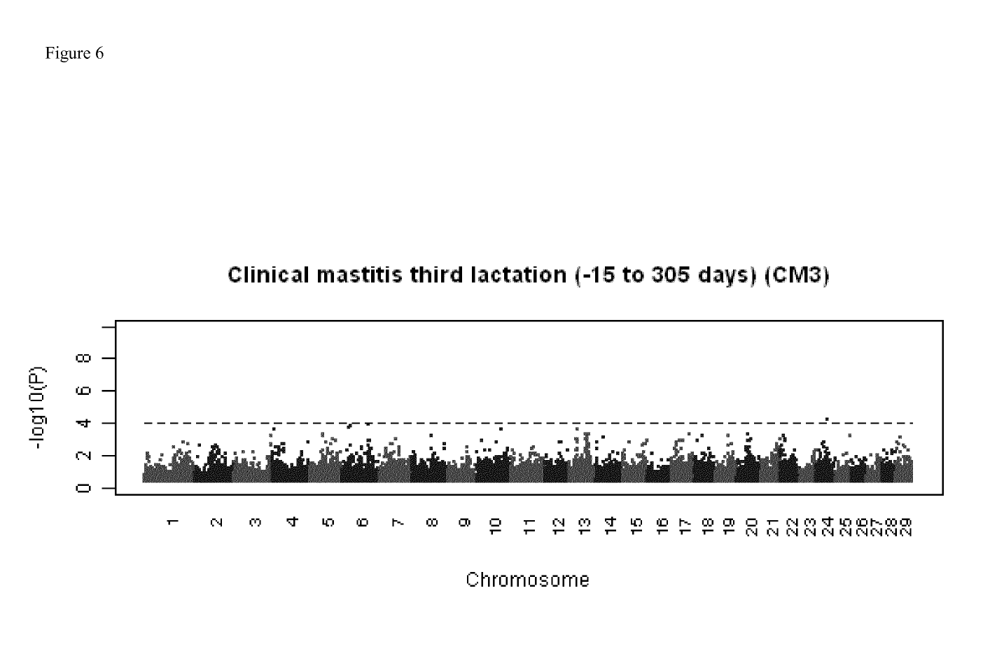

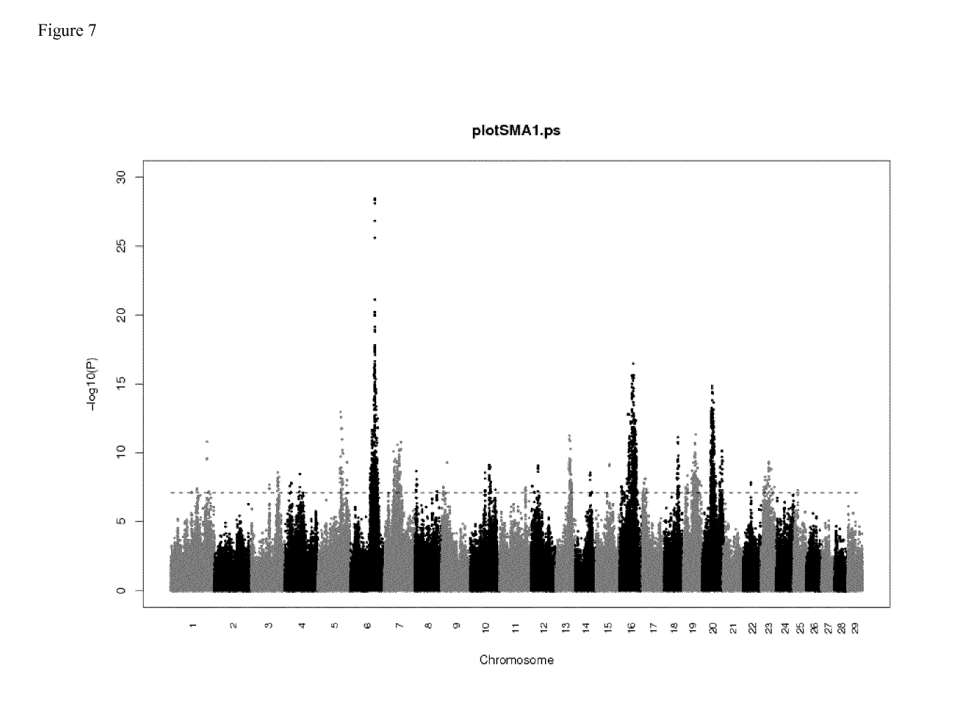

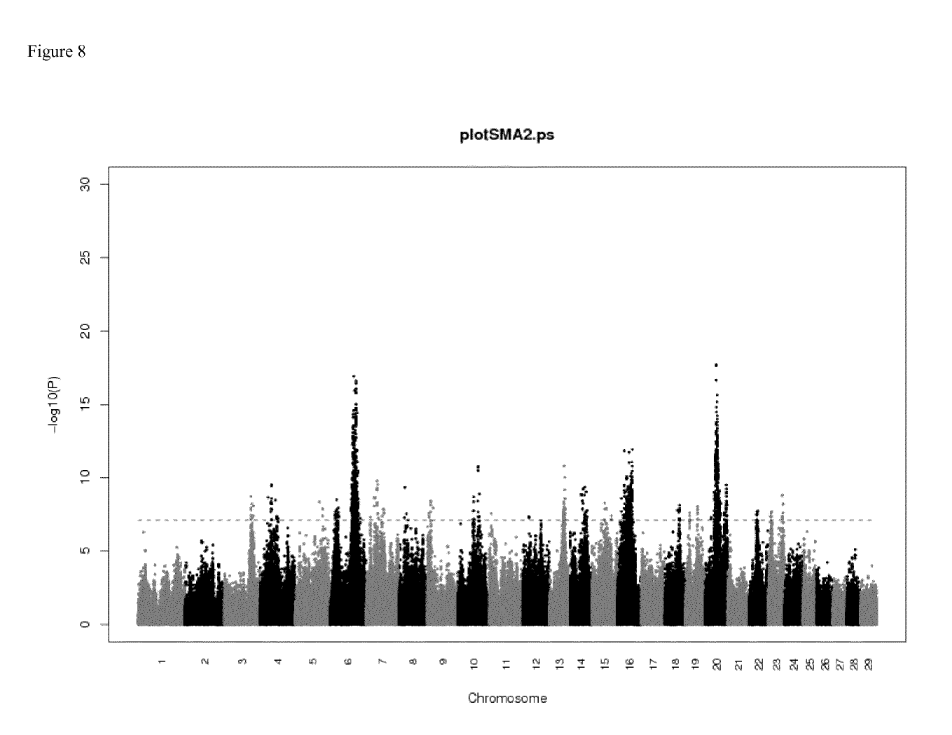

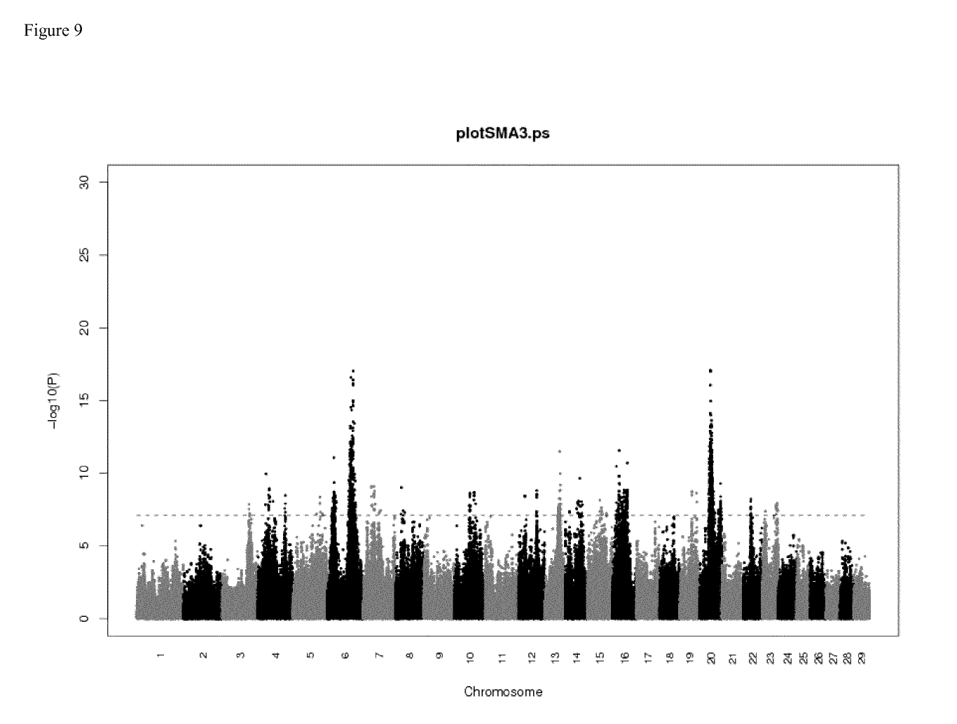

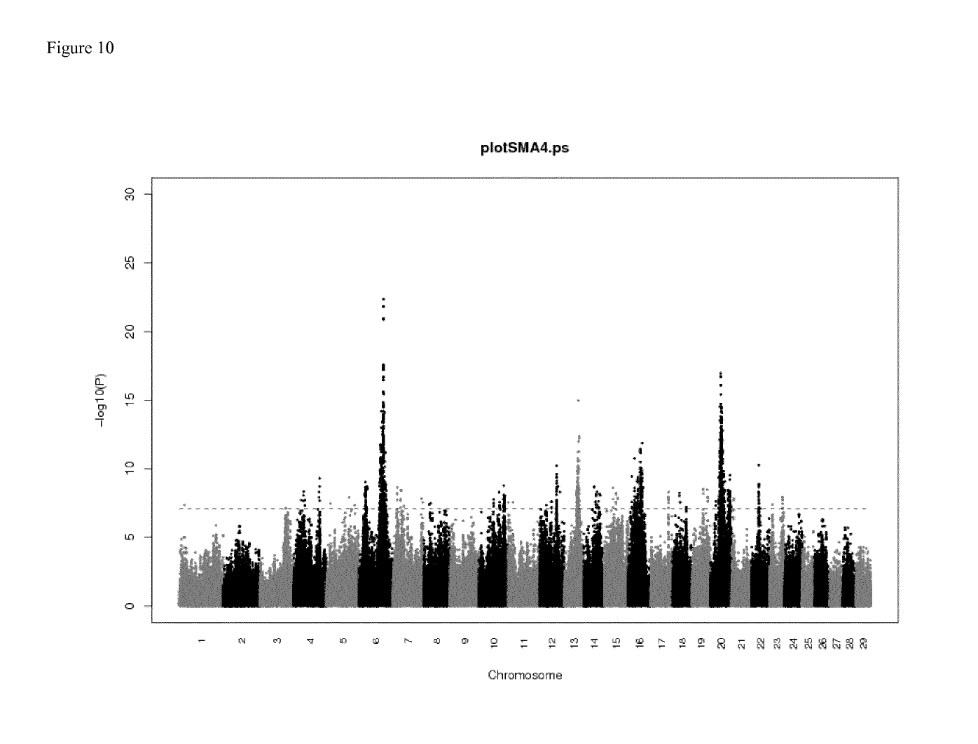

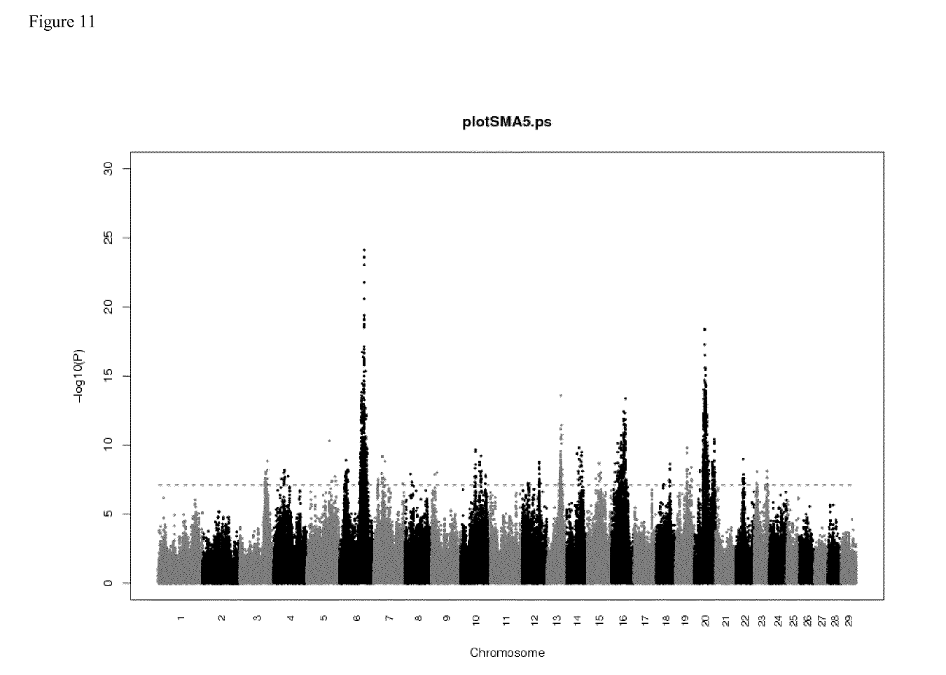

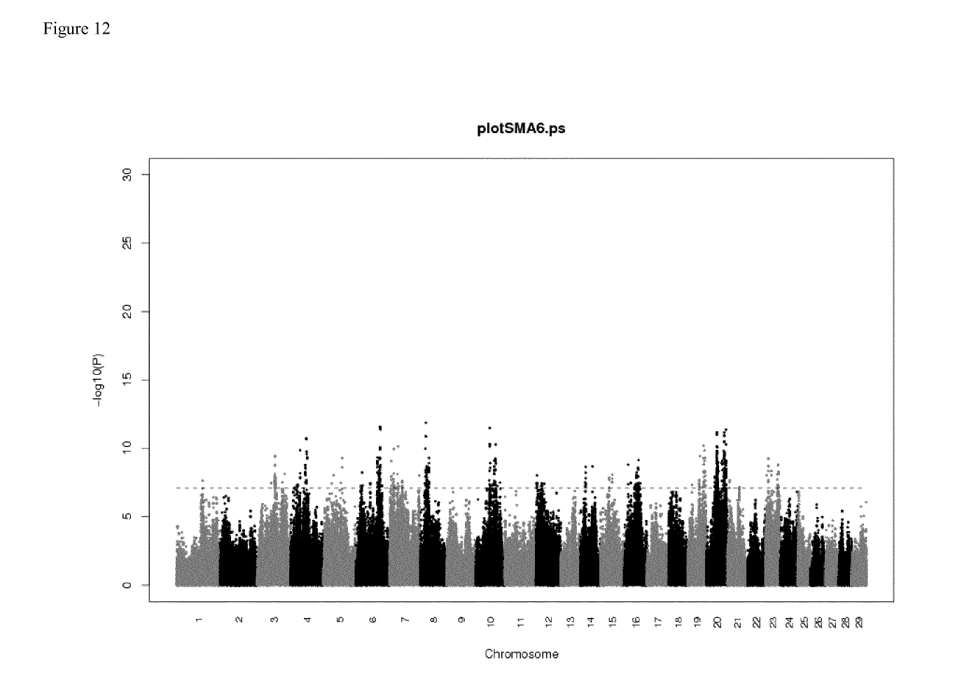

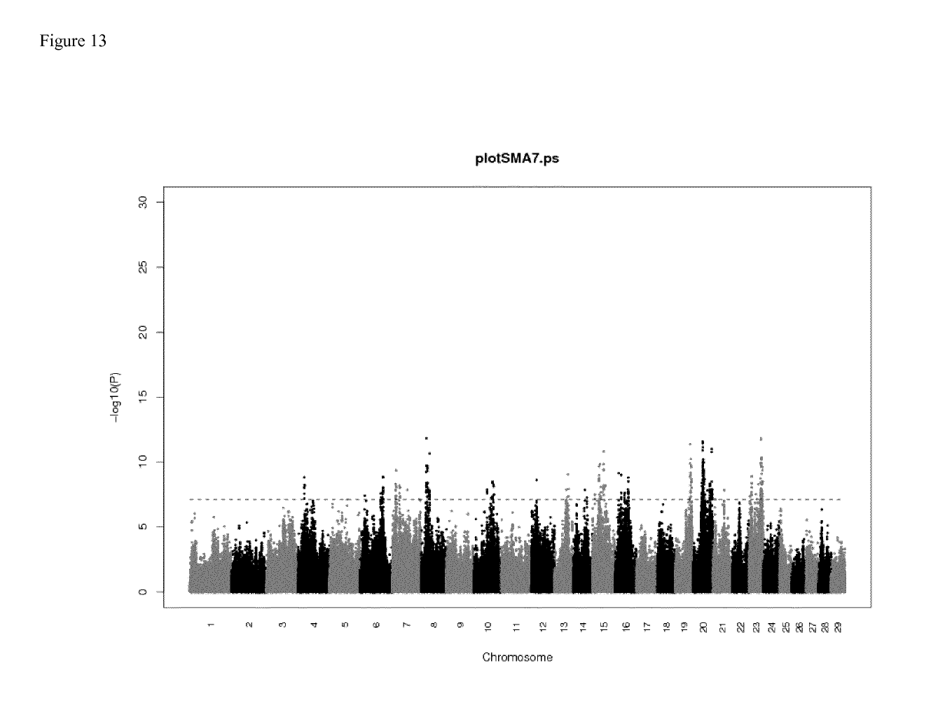

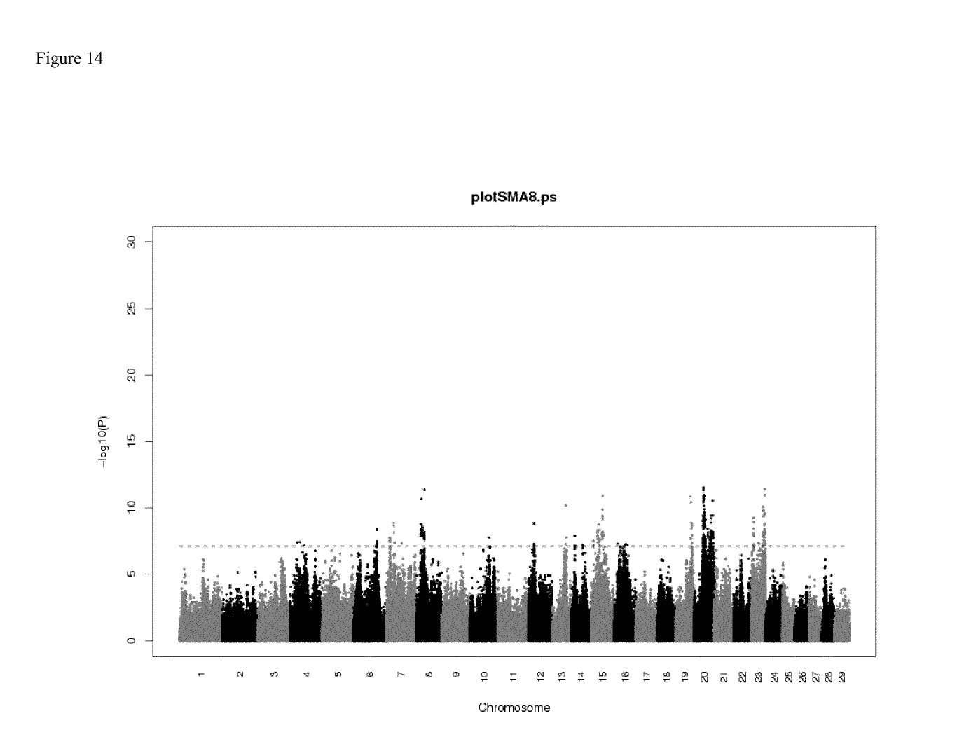

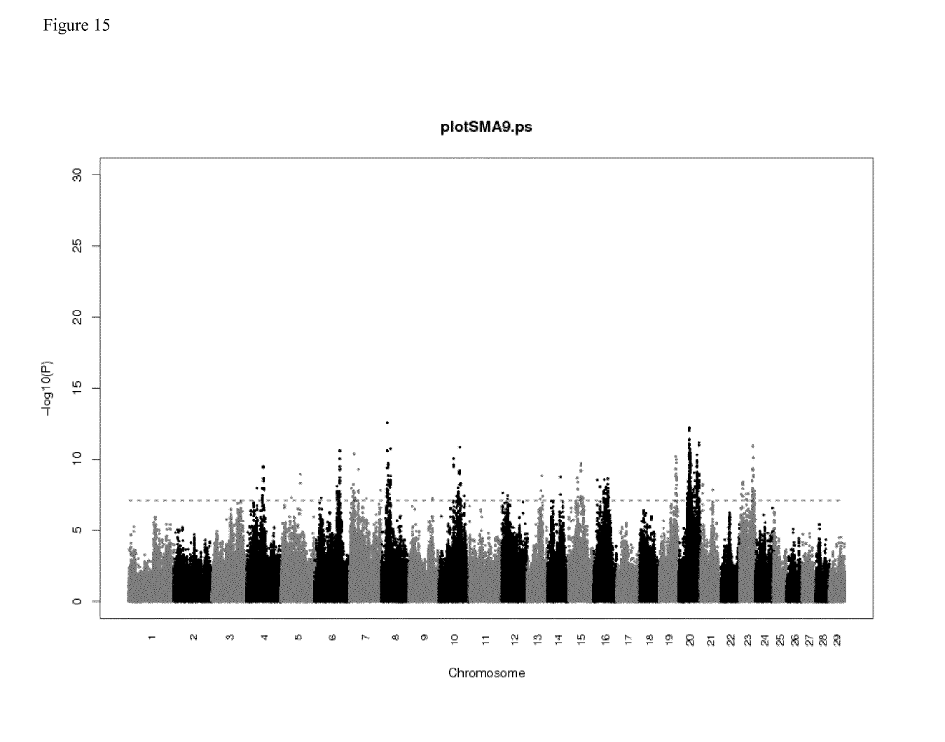

The most significant SNP for each of the nine mastitis related traits for the targeted region of BTA20 are presented in table 23. The targeted region on BTA20 was 8 Mb. The manhatton plot for this region is presented in the FIG. 6.

TABLE 23
The most significant SNP association for nine mastitis traits in the targeted region on BTA20

| | | | | | | | | Allele |
|---|---|---|---|---|---|---|---|---|
| | | | | | | | | increasing |
| | | Position | | | | -log₁₀(p- | | mastitis |
| Trait | Top-SNP | (Bp) | MAF | b-value | SE | value) | Genotype | resistance |
| CM11 | Chr20_34269660 | 34269660 | 0.457 | 2.196 | 0.297 | 12.81 | T/C | C |
| CM12 | Chr20_35965955 | 35965955 | 0.203 | 2.184 | 0.280 | 14.14 | G/A | A |
| CM2 | Chr20_35965955 | 35965955 | 0.203 | 2.344 | 0.289 | 15.24 | G/A | A |
| CM3 | Chr20_35914181 | 35914181 | 0.241 | −1.867 | 0.244 | 13.59 | G/A | G |
| CM | Chr20_35965955 | 35965955 | 0.203 | 2.095 | 0.268 | 14.17 | G/A | A |
| SCS1 | Chr20_35969130 | 35969130 | 0.315 | −1.982 | 0.272 | 12.43 | G/A | G |
| SCS2 | Chr20_35865606 | 35865606 | 0.328 | −1.861 | 0.250 | 12.98 | G/T | G |
| SCS3 | Chr20_35914086 | 35914086 | 0.086 | −22.328 | 2.938 | 13.45 | A/C | A |
| SCS | Chr20_35543794 | 35543794 | 0.323 | 1.859 | 0.250 | 12.96 | A/G | G |

Polymorphism Associated with Mastitis Resistance in the Targeted BTA20 Region:

There are two interesting candidate polymorphic variants at 35,965,955 and 35,965,956. The association results points toward the SNP at 35,965,955 which showed strong association with all the nine traits analyzed (Table 24). In ENSBTAG00000010423 there is a non-synonymous polymorphism at 35,966,158 (alt 52%). There are also candidate polymorphism at 35,942,954 (tri-alleleic indel+snp, polymorphic) and 35,942,739 (alt 52%) and a splice-site polymorphism at 35,938,178 (alt 2%). There is another non-synonymous one at 35,922,233 (alt 4%), Another gene (ENSBTAG00000019595) starts around 35,994,141. There are non-synonymous variants at, 36,011,203 (alt 84%) and 36,013,931 (alt 73%). There is a splice-site polymorphism at 36,011,211 (alt 83%). Combing the association results and functional annotation the SNP Chr20_{—}35965955 emerges as the strongest candidate polymorphism located with the targeted region on BTA20 affecting mastitis traits.

TABLE 24
The association results for the strongest polymorphism from
annotation with clinical mastitis traits on BTA20.

| | | | | | | Allele |
|---|---|---|---|---|---|---|
| | SNP | | | | | increasing |
| | position | | | -log₁₀(p- | Geno- | mastitis |
| SNP-name | (BP) | trait | MAF | value) | type | resistance |
| Chr20_35965955 | 35965955 | CM11 | 0.203 | 8.93 | G/A | A |
| Chr20_35965955 | 35965955 | CM12 | 0.203 | 14.14 | G/A | A |
| Chr20_35965955 | 35965955 | CM2 | 0.203 | 15.24 | G/A | A |
| Chr20_35965955 | 35965955 | CM3 | 0.203 | 13.25 | G/A | A |
| Chr20_35965955 | 35965955 | CM | 0.203 | 14.17 | G/A | A |
| Chr20_35965955 | 35965955 | SCS1 | 0.203 | 10.68 | G/A | A |
| Chr20_35965955 | 35965955 | SCS2 | 0.203 | 12.33 | G/A | A |
| Chr20_35965955 | 35965955 | SCS3 | 0.203 | 12.73 | G/A | A |
| Chr20_35965955 | 35965955 | SCS | 0.203 | 11.70 | G/A | A |

TABLE 25
BTA20: Genes associated with mastitis according to the present analysis.

| | Gene location | | |
|---|---|---|---|
| Ensemble Id | (UMD3.1) | Common Gene Name | Preliminary arguments |
| ENSBTAG00000010423 | 35,917,479-35,966,671 | LIFR—Leukemia Inhib- | Involved in acute phase |
| | | itory Factor Receptor | response (links to prolacti- |
| | | Alpha | noma), expressed in sali- |
| | | | va, mammary gland |
| | | | Two ns-SNPs (one with alt |
| | | | 52% in pos. 35.966.158 |
| | | | very interesting) |
| ENSBTAG00000014972 | 33,762,479-33,774,648 | Prostaglandin E2 re- | EP4R regulates intestinal |
| | | ceptor EP4 subtype | homeostasis by maintain- |
| | | | ing mucosal integrity and |
| | | | downregulating the im- |
| | | | mune response. |
| ENSBTAG00000016149 | 35,092,195-35,158,959 | Complement compo- | Complement factor |
| | | nent C9 | |
| ENSBTAG00000006697 | 35,376,524-35,514,741 | RICTOR | Components of a protein |
| | | | complex that integrates |
| | | | nutrient- and growth factor- |
| | | | derived signals to regulate |
| | | | cell growth |
| ENSBTAG00000033107 | 35,521,410-35,588,186 | OSMR—ON- | Epithelial expression, in- |
| | | COSTATIN M RE- | volved in inflammation |
| | | CEPTOR | |
| ENSBTAG00000011766 | 33,549,495-33,606,517 | Complement compo- | Complement factor |
| | | nent C7 precursor | |
| ENSBTAG00000014177 | 33,328,558-33,405,555 | complement compo- | Complement factor |
| | | nent C6 precursor | |

### Example 5

### Causative Polymorphism for BTA6 Mastitis QTL

The missense mutation, rs110326785 (G/A) in the neuropeptide FF receptor 2 gene (NPFFR2) is associated with a mastitis QTL on BTA6. This SNP located at 89,059,253 Bp (UMD3.1) causes an amino acid change 392 E to K (Glutamic acid to Lysine). The minor allele frequency of rs110326785 in Nordic Holstein is 48.3%. The allele substitution effects for nine mastitis traits in Holstein are given in the below table 26. This SNP (rs110326785) is also segregating in Nordic Red cattle population (MAF=41.2%) with allele substitution effect of −2.68 (se=0.26) for the breeding value for mastitis index and it explained 2.58% of the genetic variance. This confirms its effect in the same direction in both Holstein and Nordic Red, i.e. the allele A is reducing the resistance to mastitis in both the populations.

TABLE. 26
Effect of SNP, rs110326785, on nine mastitis
traits in Nordic Holstein population

| | | Allele substi- | | | Percent of genetic |
|---|---|---|---|---|---|
| trait | MAF | tution effect | S.E. | P-value | variance explained |
| CM11 | 0.48 | −3.16 | 0.24 | 3.93e−38 | 5.14 |
| CM12 | 0.48 | −2.43 | 0.25 | 2.11e−22 | 3.00 |
| CM2 | 0.48 | −2.54 | 0.26 | 8.30e−23 | 3.26 |
| CM3 | 0.48 | −2.77 | 0.24 | 4.52e−30 | 4.02 |
| CM-index | 0.48 | −2.82 | 0.24 | 2.39e−31 | 4.09 |
| SCS1 | 0.48 | −1.41 | 0.26 | 4.04e−08 | 0.91 |
| SCS2 | 0.48 | −1.51 | 0.27 | 2.78e−08 | 1.04 |
| SCS3 | 0.48 | −1.53 | 0.28 | 6.12e−08 | 1.10 |
| SCS-index | 0.48 | −1.49 | 0.26 | 5.03e−09 | 1.02 |

### Example 6

### Polymorphism for BTA20 Mastitis QTL

The SNP, rs133218364, is a synonymous variant within Caspase recruitment domain-containing protein 6 gene (CARD6) showed most significant association with clinical mastitis index in Holstein cattle. This SNP is located at 33,642,072 Bp on BTA20. Similarly, another SNP, rs133596506, (at 35969994 Bp) located 3323 Bp downstream to LIFR gene (Leukemia inhibitory factor receptor) also showed very high significant association with clinical mastitis index. These two variants were fitted as fixed effect in a haplotype-based analysis using 50K genotype. The variant rs133218364 was able to explain the total QTL variance for the targeted region on BTA20 (green line in the Figure below). However, rs133218364 being a synonymous variant does not change the amino acid composition of the protein. Therefore, rs133218364 is not likely the causative polymorphism underlying the QTL, but is in perfect linkage disequilibrium with the causative polymorphism. The rs133596506 located close to LIFR gene also when included in the haplotype model resulted in a substantial decrease in test statistic

Sequences

NPFFR2 gene-coding region
NCBI Reference Sequence: AC_000163.1
GenBankGraphics
>gi|258513361:89052219-89059482 Bos taurus breed Hereford
chromosome 6, Bos_taurus_UMD_3.1, whole genome shotgun sequence,
having the G-allele of the G/A SNP located at 89,059,253.
SEQ ID NO: 1
ATGAGTGAGGAATGGGATTCAAACTCTACAGAAAACTGGCATTACATTTGGAA-
TAATGCCACAACACATGATCTGTACTCAGATATCAATATTACCTATGTGAACTACTA-
TCTTCACCAGCCTCAAGTGGCAGCGATTTTCATTATTTCCTACTTTTTGATCTTCTTCC-
CTTGAACCTGGCCATAAGTGATCTACTAGTTGG-
TATATTCTGTATGCCTATCACACTGCTGGACAATATTATAGCAGGTATGTTGATCCACTCCAG-
TATTCTTGCCTGGAAAATCCCATGGATGGAGGAGCCTGGTGGGCTACAGTC-
TATGGGGTCACAAACAGCTGGAAATGACTGAGTGACTTCACTTATGTTGATTTGTG-
TACAGCTCAAAGATAATATAAAAAAATATTTGTCCCATATCCCTGCAGCTATGGTACAG-
TCATCCATTCATTTCAAATATTTACGGAGTTCCAA-
GAACTTCTCCAAGTAGCTGTCCTCATGAGGCCTACATTATAAAGGAGGA-
TAAAAAAACAACAAACAAAAAACTATATAAACAGAGAATAAAAAGAATTATGGG-
GAAAAGTAAAGCAAGTGACAGAGATGAGATGTGGAGGCTGATTTTTATAGAGTTCACTGAC-
GGTCATCCATGAATGATGACACTTCTTACTGAAGACTATGAATTTCCTTGGCAGTTCTGAG-
CACATATAGTATGGTAGGAATGTTATTGAGACTATATGCATCATAAAGCTCTAA-
GAACTGCTAAGTGTGGTTTCCATTAATATGATGTCTTCAATATAACGTAAATAGATATTTA-
GACCCTCTTGTGGTTAGCTGGGCTTCTCTGGTGGTTGGGAGATTTCCAACAGTTTTT-
GATGGAAGGCAAGCAGCAGGACCAATGATATGTCACAAAGTGGTAGTTTCATTCATGGAGTAG-
TAATTTACATGTGCAACATAAACAATGGTTCGGGTGCTACCCTAGAGGACTTCCAGGTCCG-
TATTACCACTTCCTAACACAACTTTATGTCCCTCTCTTGTGGCTCAGCTGGTAAA-
GAATCCACCTGTAATCAATCCCTGGGTTGGGAAGATTCCCCTGGAGGATGACATGG-
CAACCCACTCTAGTTTTCTTGTCTGGAAAATCCCCATGGACAGAAGAGCCTGGCAGGCTG-
CAGTCCATGGGGTCACAAACAGTTGGACACAACTGAGCGACCAAGCACAAAACATCACATTA-
TATACCCCAGAAGTATAGGAATGGTGTATCTATGGCTCCTGGTAGAGTTTTGG-
TACATAGTACCTGATTAATAAATATTTGTTGTACAAACTAATGAATAGCACTCAAGATACTCA-
TATTCCAAATCTGTATAAGAAAATATAAAAAGTATTTAGATCAAACAAGCCATATCATGGGGC-
TACTGTGGTGGCTCAGGAGTAAAGAATTTGCCTGCAATGCAGGAGATGCAGA-
GATGTGGGTTCAATCCCTGGGTCGGAAAGATTCCCTGAAGGAGAAAATGGCAACCCACTCCAG-
TAATCTTGCCCAGAAAGTAAACTGATGTTGAATGCCACAAAAGGGAAA-
GAACTGTGGTGTGGTTTGTTGTTACTGCTGTGTAGTCAGACACGACTGAGTGACTAAACAA-
TAATAACACAAGTCATATCACAGTTCTTTTCCATTATGG-
CATTCAACATAGGTTTACTGAAAAATGGAGATTTAA-
GAATTTATTTCTGTTTCTTTCCTTTCTCTGAAGTGGGAGTCAGGGAATGTTTGAGTGGC-
TATTCTATCATAATATACTACATAAATTCTGTGTTTCCATGATGCTTGTCATTTAAAAGCAA-
TATTTATTAATGATGTACATTTAAAAAAAATGATGTACATTTTTAAGATGTGCTAGACAAAAA-
GAGTTGATAAAAATTGTTGTCTCAATAAACTTAAGAAATGATCTCAATATGTCTCCCATAAA-
TATCTATAATTAAATTACTAGTTAAGTTTTTTCATATACAGTATCCTTCCCTACCCCTGAT-
TCCTATTCCCAGGAGGCAGCCACATTCAGCATTTTTGCATTTATTTTTGGTAATTACTATAA-
TATTTCTGAATAACATGTTTTTATTCTAGTATATTATCCAACTGCAGAAGATGCAATTTAG-
TTCTCATTATCCTCTTCTACCCCAAGAGATAGTTTCCCTCACCAAACTCCACTGAACTGACAG-
CACTAGGGCAAAAGAATGTAAATCCATAGAAACTGTCTGAATGTGAAATT-
GGAAAAACAACATGACTGGTTGAAATTTGGTATAAATACCAACAGACACATTTATA-
CAGAGCCACAAATATACATTCATTTTTCACCTCCCTCATTCTCTCAATATGAGCACGTCATT-
GTTTTTTGTTAAATCAATATTTAGGGTATGCATTACTATTATTATATGTAC-
CTTACCCCTGCTGAACCATGTAGAGTACTATGATAGCAAC-
CTTTTCTCTTATAAAATGTTTTTGTTTTCCTGGATTTAATAAGGGCATAATCTTTTGATTT-
GTTTAATGTTTTGAGTATAGCTATCAATAATGTTTTCTCAGATTTTCTTCCAGGAGAG-
TTAAGTTTCTTGCCAATACCTTCAAACATATAAAGTACATA-
TATGTGTTTTTAACACATCAAAAAGATGTAAATGAGGTGAATAATAAAGCTTCCAAGCTT-
GTTGTGGGGATTAGATATGTTAATAGATGCAAAATATTTATTAGAGCATATAGAATGTT-
GAAACTACTGTATAAGCTTTGACATTATTAATATACTGAAAAACAAA-
GCTCTAAATATATTAATGAAAATAATGGGAAATGTTGATTGTTCCCTGGATCTTTTAG-
GAAACAGTTACATGCATCTAATTTCATGTCTTTCTCTTCAAAATTTCAGTGAAATTAAAA-
TATACATGTATGATCTCTCTGAAGACTAACTGTTCCATTTCCCTTTCAGGATGGCCTTTT-
GGAAGTACAATGTGCAAGATCAGTGGCTTGGTTCAGGGAATATCTGTT-
GCGGCTTCTGTCTTTACTTTAGTTGCAATAGCAGTGGA-
TAGGTAGGTCAACCCCAAACTCTGAATCCAGAAAATTGAGCATGTCTGCAACTATTCTAC-
CTAACCAGTGAAAAATGTGTCATCTACTACATTTGGGCATATCTGTTTAAAATT-
GTATTCATAATATATCCTTTTATATATATATATATATGTAGTATATAATATATATACAT-
ATGCATAGTATATATGTGTGTGTATATATATTTGTGCATTATATATACATAAATT-
GTATCCACAGTATGTATCCCTTTATATATATATATATATAGAGAGAGAGAGAGAGGGAGA-
TAGGGTGTATGCATGTGCATGCTCAGTTACTCAGTCATTTCTGATTCTTTGTGAC-
CACATGGACTGTGGCCTGCCAAGTTCCTCTGTCCATGGAATTTTCTAGGCAAGAATACTG-
GAGTGGGTTGCCATTTCCTACTCCAGGG-
GATCTTCCTGAGCCAGGGATCAAACCCATGCCTCCTGCATTTGCAGGAGCATTCTTTACCAC-
TGCACCACCTGAGAAACCACACACACACACACACACACTAAGAGTTCAGTAATAAAATAAAAC-
TAGTAAAGTTTTCATATTTTAAAATTAAATAATTAGAGATGATTCATGTCCTAGTTT-
GGCCTGCTATAACAAGGTATAATACGTTATTTGATGAATGAATAAATGAAAAAATAATACTT-
GAAGTTTCCATAATTGTTTTACAAAAGGAGCAAAAATACCTAGAACAGCAC-
TATCCGTAATTTAAGGGTGAGTAAATGGGAGAATTCACTGATTAGAAGACTA-
GATGAACACTTGGAGGTTAAGACAGAAGACCTATCACTTCATGGAAAATAGATGG-
GAACAAAGTAGAAACAGTGGCAGATTTTATTTTCTTGGATTCCAAAATCACTGTG-
GATGGTGACCACAGCCACGAAATGAAATGATGCTTGCTTCTTGGAAGTTACAAGGGAA-
GCCTGGTGACAAACCTATACAGTGTATTACAAAGCAGAGACATCACTTTGTG-
GACAAAACTCACATAGTCCAACCTATGGTTTTTCCAGTAGTCCTCTAGGGATGTGAGAGTT-
GGACCATGAAGAAGGCTGAGAGCCAAAGAATTGATGCTTTAGAACTGTGCTGCTGGAGAA-
GACTCTTGAGAGTTCCTTGGACTGCAAAGAGATCAAACCAGTCAATCCTAAAGGAAATCAAC-
CGTGAATATTCATTGGAAGGACTGATGCTGAAGCTGAAACTACAATTGATGTGAAGAAC-
CAACTCATTGGAAAACACTCTGATGCTGGGAAAGATTGAGGGCAGGAGGA-
GAAGTGGGTGACAGAGAATTAGATGGTTGGAGAGCTTCACCGACTCAATGGAGATGAAATT-
GAACAAACTCTGGGAGATAGTGGAGGACAGAGAAGCCTAGCGTGGTGCAGTCCATGGGGTT-
GCAAAGAGCTGAACACAACTTAGCAACTGAGCAACAACAAAAACAAGACTTTACATATGCTTT-
GAAGGAGTTGTAAAGAAAGACAACAGAGTAGTAAAAGCTCAAGCTAACTAGTCGTTATATAAA-
GATATTAGATAAATTAGTTTGGGTTGCTTCTAAGCCATTTAAAAACTCTGTTTTCTTACCTG-
CAGATCTGGAAAACAGTAAGTTTCATAACATTTCAGTTTTATAGAGTCATCAAAAAAATCCTA-
GAAAATTCAATAGATGATAATACTTTGAAAAATGTGTTATGCAGTTGCATAGTT-
GTATGGTTATCTTATACTGCAGAAGGAAATGGCAACCCAGTCCAGTATTCTTGCCTG-
GAAAATTCCATGGAAAGAGGAGCCTGGCAGGCTACAGTTCATGAGGTCACAAAGAGTCAGA-
CATGACTGAATGACTGAGCACATGGTTATCTTATAATGAACATAATGAACATCAATAA-
TAACATTAAGAATCACAATGACAAAAATTAACAGCAGTAAAATGAACCAG-
TGTTACTCTTCATATTGATGTTGAATTTTCATGCTCCTTAGAAGATATGGAACACCAG-
GAAGGTGTATAAACAGAACTCATAATTGGCAACTCTCAGAGTCTTACAGCTCTGAAAAAAAC-
CACCAAGACACTTGGTGGCTCAAAACAGCAGTGTTCAGTACTTCCCACAACTCTG-
TAGATTGGCTGGGTGTGGTTCTCCTACTCTATGTCTTATAGCTGAAATT-
GCTCATGCTTCCACTTATACCATGCTTGCTAATGTTCAACTAACTGGCCAAAGCAAATT-
GCATGTCCAAGCACACAGATCATATGTGAGGGGACCACAGAAGGGCATGAAGGAAAGTATAA-
GAATATGGGCTCTGGAGCCAAACCACATGTGCAACAATCATGTGTGATTATGGGCAA-
GAATTTTTACCCTTTCTAAGACTTTTCCCCATAAAAGGCTTAAAGATACAATCCATGCAAAC-
CAATGAAAAGGACCTTAGAACAGAATATTAAATGTTCAATATGGGCTGCTTAACAC-
TAACATTTTTATTATAACTTTAAAATTTTTATTGGAGTAGAGTTGATTTACAATGTT-
AATACATATGCATA-
CATCCGTGCTTTTTTTCTAAAGGTTTATTGTATTTATTTATTTAATTTACTTTTT-
GGCTGTGCTGGGTCTTCGTTGCTGTGCATAGGCTTTTCTCTAACTGCAGCGAGTGGGGC-
TACTCTCCGTTGTGATGCACAGGCTTCTTGTTGCAGCAGCTTCTCTTGTTACGGAGCACAG-
GATCTAGGTGCGCAGGTTTCAGTAGCTGCAGCACATGGGCTCTGTAATT-
GTGGTTCACAGGCTCTAGACGCTGGCTCAGTAGTTGTGATGCATCAACTTAGCCACTCTGCGG-
CATGTGAGATCCTCCCAGACCAGGGATCAAACCAGCATCCCTTGCACTGCAAGACGGAT-
TCCTAATCGCTGGACCACCAGGGAAGCCTGAGTACTTTTACTATTAATAGTGTCTGATA-
TACTCCACTTATTCGTATTTTGAGTTGAAATTAATCTCATATAA-
TAATTACAGAAAATGCGTCTCTCCTAATTCTAACTTTCTACATTTTAGGGAGAACGTG-
GATGAAGACTGCAGTTACTGAAATTTAATTAATGACTCAGCCAGAAGTTATGAGCAG-
TCCTTCACTGATATTTGCCTTTCGTTACAGGTTCCGGTGTGTCATCTACCCTTTTAAAC-
CAAAGCTCACTATCAAGACGGCGTTTGTCATCATTATGATTATCTGGGTCCTGGCCATT-
GCCATCATGTCCCCATCTGCAGTAATGTTACATGTACAGGAAGAAAAAAATTACCGAGTGA-
GATTCAACTCCCAGGATAAAACCAGCCCAGTCTACTGGTGCCGGGAAGACTGGCCAAGTCAG-
GAAATGAGGAGGATTTATACCACAGTGCTGTTTGCCAATATCTAC-
CTGGCTCCCCTGTCCCTCATTGTCATCATGTATGGAAGGATTGGAATTTCACTGTTCAAGAG-
GAAAGTGCCCCACACAGGCAAACAGAACCGGGAGCAGTGGCATGTGGTATCCAAGAAGAA-
GCAGAAGATCATTAAGATGCTCCTGACCGTGGCTCTGCTTTTCATTCTCTCCTGGTT-
GCCCCTGTGGACCCTGATGATGCTCTCAGATTATGTTGACCTGTCTGCAAATGAACTG-
CAGGTCATCAATATCTACATCTACCCTTTTGCACACTGGCTGGCCTTCTGCAACAG-
CAGCGTCAACCCCATCATTTATGGTTTCTTCAATGAAAATTTTCGTCGTGGTTTCCAA-
GATGCTTTTCACCTCCAGCTCTGCCAAAAAAGAGCAAAGTCCAAGGAAGTCTACACTCTGA-
GAGCTAAAAACACTGTGGTCATCAACACATCTCATCTGTCAGCACAGGAATCAACAG-
TTAAAAACCCACACGAGGAAACTGTGCTTTGTAGGATAAGTGCTGAAAAGCCCTTACAGGAAT-
TAATGATGGAAGAATTAGGAGAAATTACCAGTAGCAATGAGATGTAAAAA-
GAGCTGGTGTGATGATTTTAACTCTGCTGTGTGATATATATTGAAATATTGTTGATGTC-
TATGGCTTCGTTCTTTAGTTCTTTCTATGAATGTTA-
GAAACCCTCTCTGAAAAAAAGTCAACAAAATGAACC
rs133218364 SNP
Original source
Variants (including SNPs and indels) imported from
dbSNP (release 137)
Alleles
Reference/Alternative: T/C|Ambiguity code: Y
Location
Chromosome 20:33640072 (forward strand)
Synonyms
None currently in the database
HGVS names
This variation has 2 HGVS names-click the plus to show
Flanking sequence
The sequence below is from the reference genome flanking the
variant location. The variant is shown in bold underlined (Y).
The Y position can be T/C. Neighbouring variants are shown
with highlighted letters and ambiguity codes (R and K).
R is a G/A variant; K is a G/T variant.
SEQ ID NO: 2
TAGATTGGGAGGACTGGGGCTGGCATGGCTTGGGCTGAGTGGATTTGGATGGCGAACTTT
CAGCTCGGGGGRCCTGGTGCTGAGTGGGCTTGGTTTGAGTGGGTTTGGGTTGAAAGGGCA
CAGGTTGGGAGGGTTTGTGCTGGGACTGTTTGACCTGGGGTGACTTGTGCTGATTAGGTC
TGAATTGAGAGCTGGGCAAATACATGCTGTAGGACATAGGATGGAATCCCATTTGGAAAG
ATGGATTTGAAGGCCCACCTTGTGTCTTCAGCTTAGCTCCTTGCTGAGGGGCAGTCCTTC
TTGGTTTTTGTGTGGCTCCTGCTGCTTGAAAGGCTTGATGATGAGGATTTTCAATATGGG
GTTTTGTTCTCATGGATGTTCTCACTGTCTCTGTTGGCTTYGTTCCCCTGGCACTGACTT
GCCCAGGCTTCCCAACTGCTCCTCCTGGCCATGAACCCAGGGAATGGAGGTGACCTACCT
GGGAGTCCTCTTTCCCAGACCTTTCAAGGGTTCCTATTGTCTGTGGTCTCTGAGGCCAGG
CCGGTATATGCTGAGACATGGGTCTTGGTGGTCTCCCAAAAGTTCTACCCATGTGATGTC
TCCAAGGAGTTCCTGAAAATCTCATAAATGTTTCACCTGAATAAAATCTCTGGGGCTGGA
AATATTGAATTCCAAAACGTTTGCCTGGACTATGTGCCCTTGTATTCTGAAAGGGCAAAG
GATGGAACCTCTTAGGCCTCTGCTGTAACCAGAAGCKGGAGCCCATAGCCCAAGGGGCTT
TCAAAGAAACATGGTTAAAGT
rs133596506 SNP
Original source
Variants (including SNPs and indels) imported from
dbSNP (release 137)
Alleles
Reference/Alternative: T/C|Ambiguity code: Y
Location
Chromosome 20:35969994 (forward strand)
Evidence status

[symbol]

Synonyms
None currently in the database
HGVS name
20:g.35969994T>C
Flanking sequence
The sequence below is from the reference genome flanking the
variant location. The variant is shown in bold underlined (Y).
The Y position can be T/C. Neighbouring variants are shown
with underlined letters and ambiguity codes (Y, R, S).
The TC underlined is a TC/-- indel variant; R is a G/A variant;
S is a G/C variant.
SEQ ID NO: 3
CCTTATTAACTGCGTATTGCATGGACTAGCATCYGTATACAATTGAAGTCTTCAGTGTGC
TAAACCTGTAGGAGCCTGGGTTTGACATTGTGGCCCAAATATCTGAATAGTTGGGTGTTT
ATGTGCTTCAGTGATAGAGGTGCTCCATCCCTGCAGTTTACACAGAGTGGCARCGATTCC
CAGAAAAATTTACAGGCAGGAGYTTCAGCCTCATTTTCCATACCAGCATTGCTTTCACGG
CTCATGGATCTGAAGGATTGCATTGAGAACATCTAGTCCTATTGCACTCTCAGAAACTGT
GGGAAAAGTCATATTCTTAAACCTTCATGCAACTTGTATTCTTGTTGGAAATTAGTCCTG
TGATTTCTTAGTTGTCTTCATACTGGCCATATTTAAAGAAYATCACAGTCCTTTTTTGTA
CTTGAATAATTAGATGTAGTTTAGTGAAGGAGACATGTGAATGTTTTCTTCCAAAAGGAA
TTTGGAATCAGTTTTAACGAGTTTGAAATAAAAGTGCTCCCTAACCTGTTAATATGCAGA
AAATATTATCTCAAATTTTTCTACTGCTGAGGCACATAATCTGATAAAACTTTTTTTTTT
TTTCTTCTGTTTAAGGTAGTTTTTACTGTTTTCTGTTCTGAACCATGTTAAAATTTGTAT
ATCTTTTATAACATASATTTCCCCCCTTATTTTGAAAGTATAAAATTGGGCATCTCAAAA
GTCAAATGTGGGATCATTAGTTAATCACTAAGACTAGGCACATAATGGAAATTCAGTCAG
GTTTTTTATTGACTGAGTCCC

1-29. (canceled)

30. A method for determining resistance to mastitis in a bovine subject, comprising detecting in a sample from said bovine subject the presence or absence of at least one genetic marker that is associated with at least one trait indicative of mastitis resistance of said bovine subject and/or offspring therefrom, wherein said at least one genetic marker is located on BTA6 in a region between 71,082,832 and 102,757,841 (UMD3.1).

31. The method according to claim 30, wherein said genetic marker is located on BTA6 in a region between 88,000,560 and 95,999,980 Mb (UMD3.1).

32. The method according to claim 30, wherein said genetic marker is located on BTA6 in the neuropeptide FF receptor 2 (NPFFR2) gene.

33. The method according to claim 30, wherein said genetic marker is located on BTA6 in the Vitamin D-binding protein precursor (GC) gene.

34. The method according to claim 30, wherein said genetic marker is located in a gene selected from the group consisting of ENSBTAG00000018531 (IGJ), ENSBTAG00000009310 (UTP3, ENSBTAG00000016795 (RUFY3), ENSBTAG00000008577 (GRSF1), ENSBTAG00000016290 (MOB1B), ENSBTAG00000012397 (DCK), ENSBTAG00000002348 (SLC4A4), ENSBTAG00000013718 (GC), ENSBTAG00000009070 (NPFFR2) and ENSBTAG00000006507 (ADAMTS3).

35. The method according to claim 30, wherein said genetic marker is selected from the group consisting of Chr6_{—}88977023, Chr6_{—}88612186, Chr6_{—}88610743, Chr6_{—}88326504, Chr6_{—}88326504, Chr6_{—}88326504, and Chr6_{—}88326504, and/or the genetic marker allele is associated with increased mastitis resistance, and/or the specific trait is as indicated in the following table:

| | | | | | | | | Allele |
|---|---|---|---|---|---|---|---|---|
| | | SNP | | | | | | increasing |
| | | position | | | | -log₁₀(p- | | mastitis |
| Trait | SNP name | (Bp) | MAF | b-value | SE | value) | Genotype | resistance |
| CM11 | Chr6_88977023 | 88977023 | 0.432 | −2.800 | 0.211 | 38.76 | C/T | C |
| CM12 | Chr6_88612186 | 88612186 | 0.403 | −2.772 | 0.262 | 25.27 | G/T | G |
| CM2 | Chr6_88610743 | 88610743 | 0.169 | −5.945 | 0.578 | 23.84 | T/A | T |
| CM3 | Chr6_88977023 | 88977023 | 0.432 | −2.447 | 0.210 | 30.21 | C/T | C |
| CM | Chr6_88977023 | 88977023 | 0.432 | −2.493 | 0.209 | 31.66 | C/T | C |
| SCS1 | Chr6_88326504 | 88326504 | 0.124 | −6.134 | 0.124 | 19.45 | G/A | G |
| SCS2 | Chr6_88326504 | 88326504 | 0.124 | −5.756 | 0.697 | 15.75 | G/A | G |
| SCS3 | Chr6_88326504 | 88326504 | 0.124 | −5.738 | 0.734 | 14.19 | G/A | G |
| SCS | Chr6_88326504 | 88326504 | 0.124 | −5.886 | 0.659 | 18.25 | G/A | G |

36. The method according to claim 30, wherein said genetic marker is the SNP BovineHD0600024355 and/or any genetic marker genetically coupled thereto.

37. The method according to claim 30, wherein said genetic marker is Chr6_{—}88977023.

38. The method according to claim 30, wherein said genetic marker is the G/A SNP located at 89,059,253 Bp (UMD3.1), wherein the A allele is associated with mastitis and the G allele is associated with resistance to mastitis.

39. The method according to claim 30, wherein said genetic marker is selected from the group consisting of the following SNPs: Chr6_{—}89059253, Chr6_{—}89059253, Chr6_{—}89059253, Chr6_{—}89059253, Chr6_{—}89059253, Chr6_{—}89059253, Chr6_{—}89059253, Chr6_{—}89059253, and Chr6_{—}89059253.

40. The method according to claim 30, wherein said trait is selected from CM11 (Clinical mastitis (1) or not (0) between −15 and 50 days after 1st calving), CM12 (Clinical mastitis (1) or not (0) between 51 and 305 days after 1st calving), CM2 (Clinical mastitis (1) or not (0) between −15 and 305 days after 2nd calving), CM3 (Clinical mastitis (1) or not (0) between −15 and 305 days after 3rd calving), CM (Clinical mastitis: 0.25*CM11+0.25*CM12+0.3*CM2+0.2*CM3), SCC1 (Log. somatic cell count average in 1st lactation), SCC2 (Log. somatic cell count average in 2nd lactation), SCC3 (Log. somatic cell count average in 3rd lactation) and SCC (Log somatic cell count: 0.5*SCC1+0.3*SCC2+0.2*SCC3).

41. The method according to claim 30, wherein the at least one genetic marker indicative of mastitis resistance is used to estimate a breeding value of said bovine subject.

42. The method according to claim 30, wherein said sample is selected from blood, semen, urine, muscle, skin, hair, ear, tail, fat, and saliva.

43. The method according to claim 30, said method comprising amplifying a genetic region comprising said genetic marker and detecting said amplification product.

44. The method according to claim 30, wherein said bovine subject is a member of the Holstein breed.

45. A method for selecting a bovine subject for breeding purposes, said method comprising determining resistance to mastitis of said bovine subject and/or offspring therefrom by a method as defined in claim 30.

46. The method according to claim 45, comprising estimating a breeding value of said selected bovine subject.

47. A method for estimating a breeding value in respect of susceptibility to mastitis in a bovine subject, comprising detecting in a sample from said bovine subject the presence or absence of at least one genetic marker that is associated with at least one trait indicative of mastitis resistance of said bovine subject and/or offspring therefrom and assigning a breeding value based on said presence or absence, wherein said at least one genetic marker is located on BTA6 in a region between 71,082,832 and 102,757,841 (UMD3.1).

48. A method for selective breeding of bovine subjects, said method comprising
providing a bovine subject,
obtaining a biological sample from said subject,
determining the presence in that sample of at least one genetic marker located on BTA6 in a region between 71,082,832 and 102,757,841 (UMD3.1),
selecting a bovine subject having in its genome said at least one genetic marker, and
using said bovine subject for breeding.

49. The method according to claim 48, said method comprising collecting semen from said selected bovine subject and using said semen for artificial insemination of one or more cows or heifers.
